(19) United States
(12) Patent Application Publication
(10) Pub. No.: US 2022/0088169 A1
(43) Pub. Date: Mar. 24, 2022

(54) VIRUS VACCINE

(71) Applicants: Griffith University, Nathan, Queensland (AU); University of Tartu, Tartu (EE)

(72) Inventors: Surendran Mahalingam, Queensland (AU); Andres Merits, Tartu (EE); Eva Zusinaite, Tartu (EE)

(73) Assignees: Griffith University, Nathan, Queensland (AU); University of Tartu, Tartu (EE)

(21) Appl. No.: 17/285,045

(22) PCT Filed: Oct. 15, 2019
(86) PCT No.: PCT/AU2019/051115

§ 371 Date: Apr. 13, 2021

(30) Foreign Application Priority Data
Oct. 16, 2018 (AU) 2018903913

(51) Int. Cl. A61K 39/12; A61P 31/14; C12N 7/00; A61K 39/00

(52) U.S. Cl. CPC A61K 39/12; A61K 2039/5254; C12N 7/00; A61P 31/14

(57) ABSTRACT

This invention relates to a vaccine comprising live attenuated Zika virus comprising a partly codon deoptimized viral genome, a Zika virus comprising a partly codon deoptimized viral genome, as well as their use in methods of treatment and prevention of viral infection. is deoptimized along the nonstructural ZIKV coding region. In some embodiments, the non-structural region of the viral genome is codon deoptimized, and preferably one or more of the genes NS1, NS2A, NS2B, NS3, NS4A, NS4B and NS5 are codon deoptimized.

### TECHNICAL FIELD

This invention generally relates to a codon deoptimized Zika virus genome. In particular, embodiments of the invention concern a vaccine comprising live attenuated Zika virus comprising a partly codon deoptimized viral genome, a Zika virus comprising a partly codon deoptimized viral genome, as well as their use in methods of treatment and prevention of viral infection.

### BACKGROUND ART

Zika virus (ZIKV) has very recently emerged as a major human pathogen (Baud D, Gubler D J, Schaub B, Lanteri M C, Musso D. An update on Zika virus infection. Lancet. 2017 Nov. 4; 390(10107):2099-2109). It is a mosquito-transmitted member of the Flavivirus genus first isolated in 1947 in Uganda from a rhesus monkey. The first human infection was recorded in 1954, but since then human infections have been reported only rarely. Since 2007 there have been a number of outbreaks in the Pacific of varying severity affecting at least 10 island nations. A particularly explosive outbreak occurred in French Polynesia in 2013 with more than 30,000 cases (Cao-Lormeau V M, Roche C, Teissier A, Robin E, Berry A L, Mallet H P, Sall A A, Musso D. Zika virus, French Polynesia, South pacific, 2013. Emerg Infect Dis. 2014 June; 20(6):1085-6; Musso D, Nilles E J, Cao-Lormeau V M. Rapid spread of emerging Zika virus in the Pacific area. Clin Microbiol Infect. 2014 October; 20(10):O595-6. doi: 10.1111/1469-0691.12707. Epub 2014 Aug. 4). ZIKV subsequently emerged and spread rapidly and extensively in the Americas, starting from 2015 (Zanluca C, Melo V C, Mosimann A L, Santos G I, Santos C N, Luz K. First report of autochthonous transmission of Zika virus in Brazil. Mem Inst Oswaldo Cruz. 2015 June; 110(4):569-72). ZIKV infections are most commonly asymptomatic. Symptomatic ZIKV infections are generally mild, with fever and rash being the dominant signs (Baud D, Gubler D J, Schaub B, Lanteri M C, Musso D. An update on Zika virus infection. Lancet. 2017 Nov. 4; 390(10107):2099-2109).

ZIKV has emerged as an important human pathogen due to its neurotropism, resulting in an increased incidence of neurological malformation, in particular, microcephaly of the developing foetus and its association with post-infectious Guillain-Barré syndrome (Kleber de Oliveira W, Cortez-Escalante J, De Oliveira W T, do Carmo G M, Henriques C M, Coelho G E, Araújo de França G V. Increase in Reported Prevalence of Microcephaly in Infants Born to Women Living in Areas with Confirmed Zika Virus Transmission During the First Trimester of Pregnancy—Brazil, 2015. MMWR Morb Mortal Wkly Rep. 2016 Mar. 11; 65(9):242-7. doi: 10.15585/mmwr.mm6509e2; Cao-Lormeau V M, Blake A, Mons S, Lastere S, Roche C, Vanhomwegen J, Dub T, Baudouin L, Teissier A, Larre P, Vial A L, Decam C, Choumet V, Halstead S K, Willison H J, Musset L, Manuguerra J C, Despres P, Foumier E, Mallet H P, Musso D, Fontanet A, Neil J, Ghawché F. Guillain; Barré Syndrome outbreak associated with Zika virus infection in French Polynesia: a case-control study. Lancet. 2016 Apr. 9; 387(10027):1531-1539. doi: 10.1016/S0140-6736(16)00562-6. Epub 2016 Mar. 2). The evidence for a causal link between ZIKV and these neurological manifestations is now very strong.

There are no licensed vaccines or antivirals available for ZIKV infection.

Codon usage bias refers to the redundancy of the genetic code, where amino acids are determined by synonymous codons that occur in different organisms at different frequencies. The process of codon optimization, where each amino acid is encoded by the most abundant codon, is frequently exploited to improve gene expression in heterologous systems, a strategy that is used to increase immune responses to antigens. Instead, codon deoptimization (CD), where each or selected number of amino acid residues is encoded by the less abundant codon, is used to decrease gene expression leading to reduced viral protein production while the composition of viral antigens remains the same. The approach can also result in additional virus attenuation by removing/altering of RNA secondary structures of functional importance (Song Y, Gorbatsevych O, Liu Y, Mugavero J, Shen S H, Ward C B, Asare E, Jiang P, Paul A V, Mueller S, Wimmer E. Limits of variation, specific infectivity, and genome packaging of massively recoded poliovirus genomes. Proc Natl Acad Sci USA. 2017 Oct. 10; 114(41): E8731-E8740. doi:10.1073/pnas.1714385114. Epub 2017 Sep. 25). This strategy is successfully used to attenuate replication of human and livestock infecting viruses (Mueller S, Papamichail D, Coleman J R, Skiena S, Wimmer E. Reduction of the rate of poliovirus protein synthesis through large-scale codon deoptimization causes attenuation of viral virulence by lowering specific infectivity. J Virol. 2006 October; 80(19):9687-96; Stobart C C, Rostad C A, Ke Z, Dillard R S, Hampton C M, Strauss J D, Yi H, Hotard A L, Meng J, Pickles R J, Sakamoto K, Lee S, Currier M G, Moin S M, Graham B S, Boukhvalova M S, Gilbert B E, Blanco J C, Piedra P A, Wright E R, Moore M L. A live RSV vaccine with engineered thermostability is immunogenic in cotton rats despite high attenuation. Nat Commun. 2016 Dec. 21; 7:13916. doi: 10.1038/ncomms13916; Diaz-San Segundo F, Medina G N, Ramirez-Medina E, Velazquez-Salinas L, Koster M, Grubman M J, de los Santos T. Synonymous Deoptimization of Foot-and-Mouth Disease Virus Causes Attenuation In Vivo while Inducing a Strong Neutralizing Antibody Response. J Virol. 2015 Nov. 18; 90(3):1298-310. doi: 10.1128/JVI.02167-15. Print 2016 Feb. 1; Baker S F, Nogales A, Martínez-Sobrido L. Downregulating viral gene expression: codon usage bias manipulation for the generation of novel influenza A virus vaccines. Future Virol. 2015 June; 10(6):715-730.; Meng J, Lee S, Hotard A L, Moore M L. Refining the balance of attenuation and immunogenicity of respiratory syncytial virus by targeted codon deoptimization of virulence genes. MBio. 2014 Sep. 23; 5(5):e01704-14. doi: 10.1128/mBio.01704-14). However, its application for arboviruses, infecting both vertebrate and mosquito host and thus adapted to replication in hosts with different codon usage is less trivial; only few examples are known (Nougairede A, De Fabritus L, Aubry F, Gould E A, Holmes E C, de Lamballerie X. Random codon re-encoding induces stable reduction of replicative fitness of Chikungunya virus in primate and mosquito cells. PLoS Pathog. 2013 February; 9(2):e1003172. doi: 10.1371/journal.ppat.1003172. Epub 2013 Feb. 21; de Fabritus L, Nougairède A, Aubry F, Gould E A, de Lamballerie X. Attenuation of tick-borne encephalitis virus using large-scale random codon re-encoding. PloS Pathog. 2015 Mar. 3; 11(3):e1004738. doi: 10.1371/journal.ppat.1004738. eCollection 2015 March; de Fabritus L, Nougairède A, Aubry F, Gould E A, de Lamballerie X. Utilisation of ISA Reverse Genetics and Large-Scale Random Codon Re-Encoding to Produce Attenuated Strains of Tick-Borne Encephalitis Virus within Days. PLoS One. 2016 Aug. 22; 11(8):e0159564. doi: 10.1371/journal.pone.0159564. eCollection 2016).

The CD method is one of several massive synonymous mutagenesis methods. Related but non-identical methods utilising different underlying principles for attenuation are codon pair bias deoptimization (Coleman J R, Papamichail D, Skiena S, Futcher B, Wimmer E, Mueller S. Virus attenuation by genome-scale changes in codon pair bias. Science. 2008 Jun. 27; 320(5884):1784-7. doi: 10.1126/science.1155761; Le Noudn C, Brock L G, Luongo C, McCarty T, Yang L, Mehedi M, Wimmer E, Mueller S, Collins P L, Buchholz U J, DiNapoli J M. Attenuation of human respiratory syncytial virus by genome-scale codon-pair deoptimization. Proc Natl Acad Sci USA. 2014 Sep. 9; 111(36):13169-74. doi: 10.1073/pnas.1411290111. Epub 2014 Aug. 25; Mueller S, Coleman J R, Papamichail D, Ward C B, Nimnual A, Futcher B, Skiena S, Wimmer E. Live attenuated influenza virus vaccines by computer-aided rational design. Nat Biotechnol. 2010 July; 28(7):723-6. doi: 10.1038/nbt.1636. Epub 2010 Jun. 13) and dinucleotide frequency modification (Atkinson N J, Witteveldt J, Evans D J, Simmonds P. The influence of CpG and UpA dinucleotide frequencies on RNA virus replication and characterization of the innate cellular pathways underlying virus attenuation and enhanced replication. Nucleic Acids Res. 2014 April; 42(7):4527-45. doi: 10.1093/nar/gku075. Epub 2014 Jan. 26). Usually these two methods are considered different from each other, though the achieved attenuation may or may not actually be the same (Futcher B, Gorbatsevych O, Shen S H, Stauft C B, Song Y, Wang B, Leatherwood J, Gardin J, Yurovsky A, Mueller S, Wimmer E. Reply to Simmonds et al. Codon pair and dinucleotide bias have not been functionally distinguished. Proc Natl Acad Sci USA. 2015 Jul. 14; 112(28):E3635-6. doi: 10.1073/pnas.1507710112. Epub 2015 Jun. 12; Tulloch F, Atkinson N J, Evans D J, Ryan M D, Simmonds P. RNA virus attenuation by codon pair deoptimisation is an artefact of increases in CpG/UpA dinucleotide frequencies. Elife. 2014 Dec. 9; 3:e04531. doi: 10.7554/eLife.04531; Simmonds P, Tulloch F, Evans D J, Ryan M D. Attenuation of dengue (and other RNA viruses) with codon pair recoding can be explained by increased CpG/UpA dinucleotide frequencies. Proc Natl Acad Sci USA. 2015 Jul. 14; 112(28):E3633-4). Clearly, however, these methods are very different from CD.

### SUMMARY OF THE INVENTION

According to a first embodiment of the present invention, there is provided live attenuated recombinant Zika virus, recombinant Zika virus, recombinant Zika virus particle or recombinant Zika virus nucleic acid comprising a partly codon deoptimized Zika viral genome.

According to a second embodiment of the present invention, there is provided a recombinant, isolated or substantially purified nucleic acid comprising a partly codon deoptimized Zika viral genome or partly codon deoptimized region thereof.

According to a third embodiment of the present invention, there is provided a vector containing the nucleic acid of the second embodiment.

According to a fourth embodiment of the present invention, there is provided a cell or isolate containing the live attenuated recombinant Zika virus, recombinant Zika virus, recombinant Zika virus particle or recombinant Zika virus nucleic acid of the first embodiment, the nucleic acid of the second embodiment, or the vector of the third embodiment.

According to a fifth embodiment of the present invention, there is provided a vaccine comprising the live attenuated recombinant Zika virus, recombinant Zika virus, recombinant Zika virus particle or recombinant Zika virus nucleic acid of the first embodiment, the recombinant, isolated or substantially purified nucleic acid of the second embodiment, the vector of the third embodiment, or the cell or isolate of the fourth embodiment.

According to a sixth embodiment of the present invention, there is provided a pharmaceutical preparation comprising the live attenuated recombinant Zika virus, recombinant Zika virus, recombinant Zika virus particle or recombinant Zika virus nucleic acid of the first embodiment, the recombinant, isolated or substantially purified nucleic acid of the second embodiment, the vector of the third embodiment, or the cell or isolate of the fourth embodiment.

According to a seventh embodiment of the present invention, there is provided an immunogenic composition comprising the live attenuated recombinant Zika virus, recombinant Zika virus, recombinant Zika virus particle or recombinant Zika virus nucleic acid of the first embodiment, the recombinant, isolated or substantially purified nucleic acid of the second embodiment, the vector of the third embodiment, or the cell or isolate of the fourth embodiment.

According to an eighth embodiment of the present invention, there is provided a method of (1) treating a subject having a natural Zika viral infection, (2) reducing the severity of a natural Zika viral infection in a subject, or (3) preventing a subject from contracting a Zika viral infection naturally, said method comprising the step of administering to the subject: the live attenuated recombinant Zika virus, recombinant Zika virus, recombinant Zika virus particle or recombinant Zika virus nucleic acid of the first embodiment; the recombinant, isolated or substantially purified nucleic acid of the second embodiment; the vector of the third embodiment; the cell or isolate of the fourth embodiment; the vaccine of the fifth embodiment; the pharmaceutical preparation of the sixth embodiment; or the immunogenic composition of the seventh embodiment.

According to a ninth embodiment of the present invention, there is provided use of: the live attenuated recombinant Zika virus, recombinant Zika virus, recombinant Zika virus particle or recombinant Zika virus nucleic acid of the first embodiment; the recombinant, isolated or substantially purified nucleic acid of the second embodiment; the vector of the third embodiment; the cell or isolate of the fourth embodiment; the vaccine of the fifth embodiment; the pharmaceutical preparation of the sixth embodiment; or the immunogenic composition of the seventh embodiment, in the preparation of a medicament for (1) treating a subject having a natural Zika viral infection, (2) reducing the severity of a natural Zika viral infection in a subject, or (3) preventing a subject from contracting a Zika viral infection naturally.

According to a tenth embodiment of the present invention, there is provided: a live attenuated recombinant Zika virus, recombinant Zika virus, recombinant Zika virus particle or recombinant Zika virus nucleic acid of the first embodiment; a recombinant, isolated or substantially purified nucleic acid of the second embodiment; a vector of the third embodiment; a cell or isolate of the fourth embodiment; a vaccine of the fifth embodiment; a pharmaceutical preparation of the sixth embodiment; or an immunogenic composition of the seventh embodiment, for use in (1) treating a subject having a natural Zika viral infection, (2) reducing the severity of a natural Zika viral infection in a subject, or (3) preventing a subject from contracting a Zika viral infection naturally.

According to an eleventh embodiment of the present invention, there is provided a method of generating a live attenuated Zika virus vaccine, recombinant Zika virus, recombinant Zika virus particle or recombinant Zika virus nucleic acid, or recombinant, isolated or substantially purified nucleic acid comprising a partly codon deoptimized Zika viral genome or partly codon deoptimized region thereof, comprising the step of partly codon deoptimizing a Zika viral genome.

According to a twelfth embodiment of the present invention, there is provided a method of preparing a vaccine comprising live attenuated recombinant Zika virus, said method comprising the steps of: (1) codon deoptimizing a Zika viral genome to produce a partly codon deoptimized live attenuated Zika virus; and (2) enabling the partly codon deoptimized live attenuated Zika virus to replicate.

### BRIEF DESCRIPTION OF THE DRAWINGS

FIG. 1a. Schematic representation of codon deoptimized ZIKV genomes. ZIKV-DO—amino acid codons in NS1-NS2A-NS2B-NS3 regions are maximally deoptimized; ZIKV-DO-NS3—amino acid codons in NS3 region are maximally deoptimized; ZIKV-DO-scattered—amino acid codons are deoptimized with 3-4 codon gaps through all the nonstructural ZIKV genome region. In ZIKV-scatter genome every 3^{rd}-4^{th} amino acid codon has been deoptimized in favour of rarely used codons. Grey area—ZIKV structural region encompassing C, prM and E (unchanged); white area/s—unchanged; nonstructural region, pink area labelled ‘CD modified’—codon deoptimized region.

FIG. 1b. An example of computational codon deoptimization of the nonstructural ZIKV region [SEQ ID NO:1], with changes indicated by way of underlining and insertion arrows.

FIG. 2. Graph showing the percentage survival of mice infected intracranially with Zika wt virus (MR 766) or Zika vaccine based on clone ZIKV-DO-NS3 over 15 days post infection. The graph shows that the vaccine comprising live-attenuated codon deoptimized Zika virus (based on ZIKV-DO-NS3) did not result in lethal infection in mice as compared to Zika wt virus.

FIG. 3. A. Graph showing the clinical score of mice infected intracranially with Zika wt virus (MR 766) or Zika vaccine based on clone ZIKV-DO-NS3 over 15 days post infection. B. Graph showing the body weight of mice infected intracranially with Zika wt virus (MR 766) or Zika vaccine based on clone ZIKV-DO-NS3 over 15 days post infection. The graphs show that the vaccine comprising live-attenuated codon deoptimized Zika virus based on ZIKV-DO-NS3 did not show signs of disease nor weight loss.

FIG. 4. Graph showing the percentage survival of mice either vaccinated with a Zika vaccine based on clone ZIKV-DO-NS3 or not, and challenged with Zika wt virus (MR 766), over 15 days post infection. The graph shows that vaccinated mice were fully protected from lethal infection with no mortality.

FIG. 5. Graph showing the body weight loss of mice either vaccinated with Zika vaccine based on clone ZIKV-DO-NS3 or not, and challenged with Zika wt virus (MR 766), over 7 days post infection. The graph shows that vaccinated mice were fully protected from lethal infection with no weight loss.

FIG. 6. A. Clinical score criteria used for the graph shown in B. B. Graph showing the clinical score of mice either vaccinated with Zika vaccine based on clone ZIKV-DO-NS3 or not, and challenged with Zika wt virus (MR 766), over 6 days post infection. The graph shows that vaccinated mice were fully protected from lethal infection with no disease signs.

FIG. 7. Graph showing the level (PFU/IFU) of Zika virus in brain tissue of mice either vaccinated with Zika vaccine based on clone ZIKV-DO-NS3 or not, and challenged with Zika wt virus. The graph shows that there was no detectable virus in the brains of vaccinated mice at day 6 after Zika challenge.

FIG. 8. Graph showing that a vaccine based on ZIKV-DO-NS3 that is given subcutaneously to mice can induce a cellular response in the lymph nodes, as compared with a naïve non-vaccinated mouse group.

FIG. 9. Graph showing that a vaccine based on ZIKV-DO-NS3 that is given to mice induced a strong ZIKV antibody response, as compared with a naïve non-vaccinated mouse group.

FIG. 10. Graph showing a vaccine based on ZIKV-DO-NS3 induced a strong ZIKV neutralising antibody response in mice as compared with a naïve non-vaccinated mouse group.

FIG. 11. Graphs showing that a vaccine based on ZIKV-DO-NS3 that is given subcutaneously to mice can induce an immune response (B and T cell response) in the draining lymph nodes compared with a naïve non-vaccinated mouse group.

### DESCRIPTION OF SEQUENCES

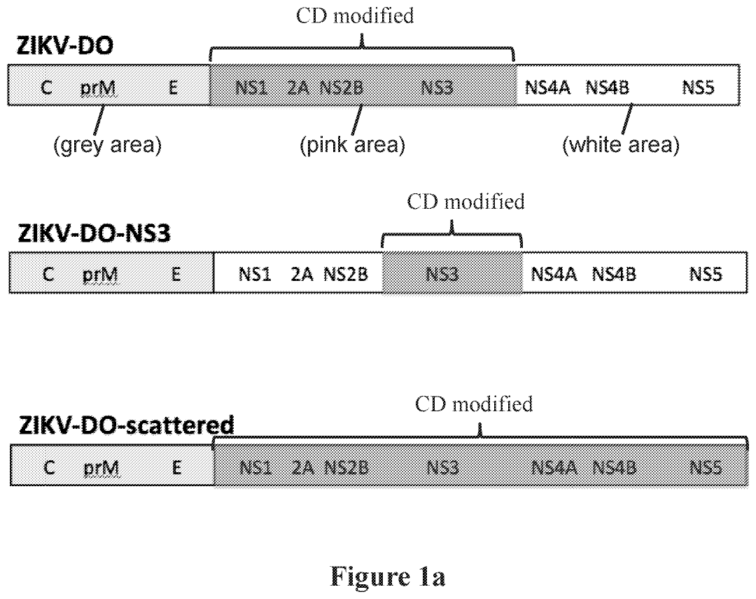

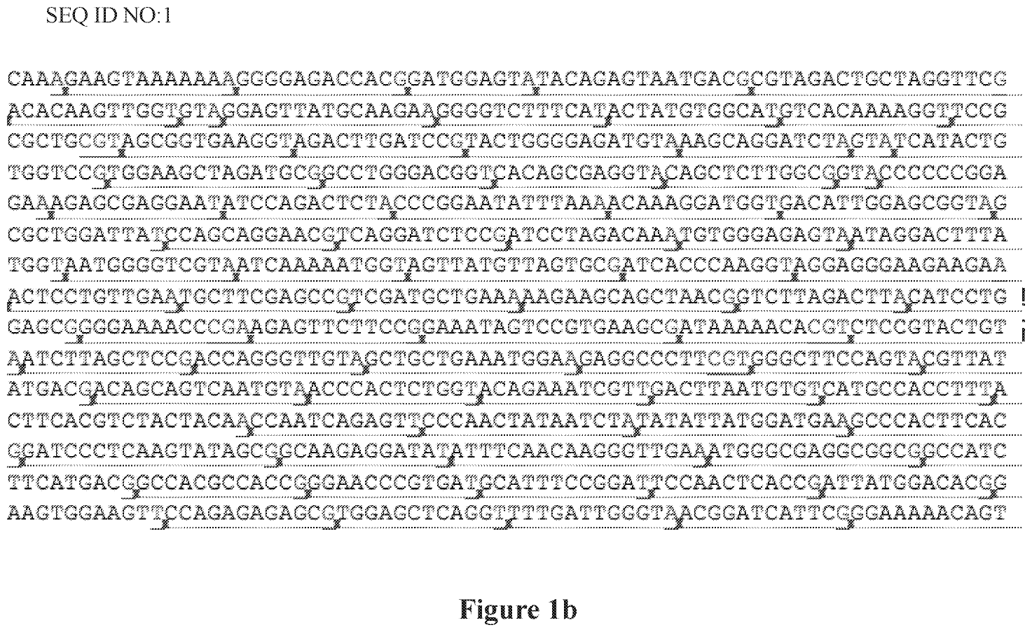

SEQ ID NO:1. See FIG. 1b. Computational codon deoptimization of a nonstructural ZIKV region, with changes indicated by way of underlining and insertion arrows. SEQ ID NO:1, below, with changed nucleotides marked in bold and underline. The sequence derives from the ZIKV-DO-scattered vaccine candidate, in particular the ZIKV-DO-scattered NS3 region.

CAAAGAAGTAAAAAAAGGGGAGACCACGGATGGAGTATACAGAGTAATGACGCGTAGAC
TGCTAGGTTCGACACAAGTTGGTGTAGGAGTTATGCAAGAAGGGGTCTTTCATACTATGTGGCATGTC
ACAAAAGGTTCCGCGCTGCGTAGCGGTGAAGGTAGACTTGATCCGTACTGGGGAGATGTAAAGCAGGA
TCTAGTATCATACTGTGGTCCGTGGAAGCTAGATGCGGCCTGGGACGGTCACAGCGAGGTACAGCTCT
TGGCGGTACCCCCCGGAGAAAGAGCGAGGAATATCCAGACTCTACCCGGAATATTTAAAACAAAGGAT
GGTGACATTGGAGCGGTAGCGCTGGATTATCCAGCAGGAACGTCAGGATCTCCGATCCTAGACAAATG
TGGGAGAGTAATAGGACTTTATGGTAATGGGGTCGTAATCAAAAATGGTAGTTATGTTAGTGCGATCA
CCCAAGGTAGGAGGGAAGAAGAAACTCCTGTTGAATGCTTCGAGCCGTCGATGCTGAAAAAGAAGCAG
CTAACGGTCTTAGACTTACATCCTGGAGCGGGGAAAACCCGAAGAGTTCTTCCGGAAATAGTCCGTGA
AGCGATAAAAACACGTCTCCGTACTGTAATCTTAGCTCCGACCAGGGTTGTAGCTGCTGAAATGGAAG
AGGCCCTTCGTGGGCTTCCAGTACGTTATATGACGACAGCAGTCAATGTAACCCACTCTGGTACAGAA
ATCGTTGACTTAATGTGTCATGCCACCTTTACTTCACGTCTACTACAACCAATCAGAGTTCCCAACTA
TAATCTATATATTATGGATGAAGCCCACTTCACGGATCCCTCAAGTATAGCGGCAAGAGGATATATTT
CAACAAGGGTTGAAATGGGCGAGGCGGCGGCCATCTTCATGACGGCCACGCCACCGGGAACCCGTGAT
GCATTTCCGGATTCCAACTCACCGATTATGGACACGGAAGTGGAAGTTCCAGAGAGAGCGTGGAGCTC
AGGTTTTGATTGGGTAACGGATCATTCGGGAAAAACAGT

SEQ ID NO:2. ZIKV-wild type nonstructural region nucleotide sequence, with locations of nonstructural regions NS1 to NS5 indicated.

(NS1)GATGTGGGGTGCTCGGTGGACTTCTCAAAGAAGGAGACGAGATGCGGTACAGGG
GTGTTCGTCTATAACGACGTTGAAGCCTGGAGGGACAGGTACAAGTACCATCCTGACTCCCCCCGTAG
ATTGGCAGCAGCAGTCAAGCAAGCCTGGGAAGATGGTATCTGCGGGATCTCCTCTGTTTCAAGAATGG
AAAACATCATGTGGAGATCAGTAGAAGGGGAGCTCAACGCAATCCTGGAAGAGAATGGAGTTCAACTG
ACGGTCGTTGTGGGATCTGTAAAAAACCCCATGTGGAGAGGTCCACAGAGATTGCCCGTGCCTGTGAA
CGAGCTGCCCCACGGCTGGAAGGCTTGGGGGAAATCGTACTTCGTCAGAGCAGCAAAGACAAATAACA
GCTTTGTCGTGGATGGTGACACACTGAAGGAATGCCCACTCAAACATAGAGCATGGAACAGCTTTCTT
GTGGAGGATCATGGGTTCGGGGTATTTCACACTAGTGTCTGGCTCAAGGTTAGAGAAGATTATTCATT
AGAGTGTGATCCAGCCGTTATTGGAACAGCTGTTAAGGGAAAGGAGGCTGTACACAGTGATCTAGGCT
ACTGGATTGAGAGTGAGAAGAATGACACATGGAGGCTGAAGAGGGCCCATCTGATCGAGATGAAAACA
TGTGAATGGCCAAAGTCCCACACATTGTGGACAGATGGAATAGAAGAGAGTGATCTGATCATACCCAA
GTCTTTAGCTGGGCCACTCAGCCATCACAATACCAGAGAGGGCTACAGGACCCAAATGAAAGGGCCAT
GGCACAGTGAAGAGCTTGAAATTCGGTTTGAGGAATGCCCAGGCACTAAGGTCCACGTGGAGGAAACA
TGTGGAACAAGAGGACCATCTCTGAGATCAACCACTGCAAGCGGAAGGGTGATCGAGGAATGGTGCTG
CAGGGAGTGCACAATGCCCCCACTGTCGTTCCGGGCTAAAGATGGCTGTTGGTATGGAATGGAGATAA
GGCCCAGGAAAGAACCAGAAAGCAACTTAGTAAGGTCAATGGTGACTGCA(NS2A)GGATCAACTGAT
CACATGGACCACTTCTCCCTTGGAGTGCTTGTGATCCTGCTCATGGTGCAGGAAGGGCTGAAGAAGAG
AATGACCACAAAGATCATCATAAGCACATCAATGGCAGTGCTGGTAGCTATGATCCTGGGAGGATTTT
CAATGAGTGACCTGGCTAAGCTTGCAATTTTGATGGGTGCCACCTTCGCGGAAATGAACACTGGAGGA
GATGTAGCTCATCTGGCGCTGATAGCGGCATTCAAAGTCAGACCAGCGTTGCTGGTATCTTTCATCTT
CAGAGCTAATTGGACACCCCGTGAAAGCATGCTGCTGGCCTTGGCCTCGTGTCTTTTGCAAACTGCGA
TCTCCGCCTTGGAAGGCGACCTGATGGTTCTCATCAATGGTTTTGCTTTGGCCTGGTTGGCAATACGA
GCGATGGTTGTTCCACGCACTGATAACATCACCTTGGCAATCCTGGCTGCTCTGACACCACTGGCCCG
GGGCACACTGCTTGTGGCGTGGAGAGCAGGCCTTGCTACTTGCGGGGGGTTTATGCTCCTCTCTCTGA
AGGGAAAAGGCAGTGTGAAGAAGAACTTACCATTTGTCATGGCCCTGGGACTAACCGCTGTGAGGCTG
GTCGACCCCATCAACGTGGTGGGACTGCTGTTACTCACAAGGAGTGGGAAGCGG(NS2B)AGCTGGCC
CCCTAGCGAAGTACTCACAGCTGTTGGCCTGATATGCGCATTGGCTGGAGGGTTCGCCAAGGCAGATA
TAGAGATGGCTGGGCCCATGGCCGCGGTCGGTCTGCTAATTGTCAGTTACGTGGTCTCAGGAAAGAGT
GTGGACATGTACATTGAAAGAGCAGGTGACATCACATGGGAAAAAGATGCGGAAGTCACTGGAAACAG
TCCCCGGCTCGATGTGGCGCTAGATGAGAGTGGTGATTTCTCCCTGGTGGAGGATGACGGTCCCCCCA
TGAGAGAGATCATACTCAAGGTGGTCCTGATGACCATCTGTGGCATGAATCCAATAGCCATACCCTTT
GCAGCTGGAGCGTGGTACGTATACGTGAAGACTGGAAAAAGG(NS3)AGTGGTGCTCTATGGGATGTG
CCTGCTCCCAAGGAAGTAAAAAAGGGGGAGACCACAGATGGAGTGTACAGAGTAATGACTCGTAGACT
GCTAGGTTCAACACAAGTTGGAGTGGGAGTTATGCAAGAGGGGGTCTTTCACACTATGTGGCACGTCA
CAAAAGGATCCGCGCTGAGAAGCGGTGAAGGGAGACTTGATCCATACTGGGGAGATGTCAAGCAGGAT
CTGGTGTCATACTGTGGTCCATGGAAGCTAGATGCCGCCTGGGACGGGCACAGCGAGGTGCAGCTCTT
GGCCGTGCCCCCCGGAGAGAGAGCGAGGAACATCCAGACTCTGCCCGGAATATTTAAGACAAAGGATG
GGGACATTGGAGCGGTTGCGCTGGATTACCCAGCAGGAACTTCAGGATCTCCAATCCTAGACAAGTGT
GGGAGAGTGATAGGACTTTATGGCAATGGGGTCGTGATCAAAAATGGGAGTTATGTTAGTGCCATCAC
CCAAGGGAGGAGGGAAGAAGAGACTCCTGTTGAGTGCTTCGAGCCCTCGATGCTGAAGAAGAAGCAGC
TAACTGTCTTAGACTTGCATCCTGGAGCTGGGAAAACCAGGAGAGTTCTTCCTGAAATAGTCCGTGAA
GCCATAAAAACAAGACTCCGTACTGTGATCTTAGCTCCAACCAGGGTTGTCGCTGCTGAAATGGAGGA
GGCCCTTAGAGGGCTTCCAGTGCGTTATATGACAACAGCAGTCAATGTCACCCACTCTGGAACAGAAA
TCGTCGACTTAATGTGCCATGCCACCTTCACTTCACGTCTACTACAGCCAATCAGAGTCCCCAACTAT
AATCTGTATATTATGGATGAGGCCCACTTCACAGATCCCTCAAGTATAGCAGCAAGAGGATACATTTC
AACAAGGGTTGAGATGGGCGAGGCGGCTGCCATCTTCATGACCGCCACGCCACCAGGAACCCGTGACG
CATTTCCGGACTCCAACTCACCAATTATGGACACCGAAGTGGAAGTCCCAGAGAGAGCCTGGAGCTCA
GGCTTTGATTGGGTGACGGATCATTCTGGAAAAACAGTTTGGTTTGTTCCAAGCGTGAGGAACGGCAA
TGAGATCGCAGCTTGTCTGACAAAGGCTGGAAAACGGGTCATACAGCTCAGCAGAAAGACTTTTGAGA
CAGAGTTCCAGAAAACAAAACATCAAGAGTGGGACTTTGTCGTGACAACTGACATTTCAGAGATGGGC
GCCAACTTTAAAGCTGACCGTGTCATAGATTCCAGGAGATGCCTAAAGCCGGTCATACTTGATGGCGA
GAGAGTCATTCTGGCTGGACCCATGCCTGTCACACATGCCAGCGCTGCCCAGAGGAGGGGGCGCATAG
GCAGGAATCCCAACAAACCTGGAGATGAGTATCTGTATGGAGGTGGGTGCGCAGAGACTGACGAAGAC
CATGCACACTGGCTTGAAGCAAGAATGCTCCTTGACAATATTTACCTCCAAGATGGCCTCATAGCCTC
GCTCTATCGACCTGAGGCCGACAAAGTAGCAGCCATTGAGGGAGAGTTCAAGCTTAGGACGGAGCAAA
GGAAGACCTTTGTGGAACTCATGAAAAGAGGAGATCTTCCTGTTTGGCTGGCCTATCAGGTTGCATCT
GCCGGAATAACCTACACAGATAGAAGATGGTGCTTTGATGGCACGACCAACAACACCATAATGGAAGA
TAGTGTGCCGGCAGAGGTGTGGACCAGACACGGAGAGAAAAGAGTGCTCAAACCGAGGTGGATGGACG
CCAGAGTTTGTTCAGATCATGCGGCCCTGAAGTCATTCAAGGAGTTTGCCGCTGGGAAAAGA(NS4A)
GGAGCGGCTTTTGGAGTGATGGAAGCCCTGGGAACACTGCCAGGACACATGACAGAGAGATTCCAGGA
AGCCATTGACAACCTCGCTGTGCTCATGCGGGCAGAGACTGGAAGCAGGCCTTACAAAGCCGCGGCGG
CCCAATTGCCGGAGACCCTAGAGACCATAATGCTTTTGGGGTTGCTGGGAACAGTCTCGCTGGGAATC
TTCTTCGTCTTGATGAGGAACAAGGGCATAGGGAAGATGGGCTTTGGAATGGTGACTCTTGGGGCCAG
CGCATGGCTCATGTGGCTCTCGGAAATTGAGCCAGCCAGAATTGCATGTGTCCTCATTGTTGTGTTCC
TATTGCTGGTGGTGCTCATACCTGAGCCAGAAAAGCAAAGATCTCCCCAGGACAACCAAATGGCAATC
ATCATCATGGTAGCAGTAGGTCTTCTGGGCTTGATTACCGCC(NS4B)AATGAACTCGGATGGTTGGA
GAGAACAAAGAGTGACCTAAGCCATCTAATGGGAAGGAGAGAGGAGGGGGCAACCATAGGATTCTCAA
TGGACATTGACCTGCGGCCAGCCTCAGCTTGGGCCATCTATGCTGCCTTGACAACTTTCATTACCCCA
GCCGTCCAACATGCAGTGACCACCTCATACAACAACTACTCCTTAATGGCGATGGCCACGCAAGCTGG
AGTGTTGTTTGGTATGGGCAAAGGGATGCCATTCTACGCATGGGACTTTGGAGTCCCGCTGCTAATGA
TAGGTTGCTACTCACAATTAACACCCCTGACCCTAATAGTGGCCATCATTTTGCTCGTGGCGCACTAC
ATGTACTTGATCCCAGGGCTGCAGGCAGCAGCTGCGCGTGCTGCCCAGAAGAGAACGGCAGCTGGCAT
CATGAAGAACCCTGTTGTGGATGGAATAGTGGTGACTGACATTGACACAATGACAATTGACCCCCAAG
TGGAGAAAAAGATGGGACAGGTGCTACTCATAGCAGTAGCCGTCTCCAGCGCCATACTGTCGCGGACC
GCCTGGGGGTGGGGGGAGGCTGGGGCCCTGATCACAGCCGCAACTTCCACTTTGTGGGAAGGCTCTCC
GAACAAGTACTGGAACTCCTCTACAGCCACTTCACTGTGTAACATTTTTAGGGGAAGTTACTTGGCTG
GAGCTTCTCTAATCTACACAGTAACAAGAAACGCTGGCTTGGTCAAGAGACGT(NS5)GGGGGTGGAA
CAGGAGAGACCCTGGGAGAGAAATGGAAGGCCCGCTTGAACCAGATGTCGGCCCTGGAGTTCTACTCC
TACAAAAAGTCAGGCATCACCGAGGTGTGCAGAGAAGAGGCCCGCCGCGCCCTCAAGGACGGTGTGGC
AACGGGAGGCCATGCTGTGTCCCGAGGAAGTGCAAAGCTGAGATGGTTGGTGGAGCGGGGATACCTGC
AGCCCTATGGAAAGGTCATTGATCTTGGATGTGGCAGAGGGGGCTGGAGTTACTACGCCGCCACCATC
CGCAAAGTTCAAGAAGTGAAAGGATACACAAAAGGAGGCCCTGGTCATGAAGAACCCGTGTTGGTGCA
AAGCTATGGGTGGAACATAGTCCGTCTTAAGAGTGGGGTGGACGTCTTTCATATGGCGGCTGAGCCGT
GTGACACGCTGCTGTGTGACATAGGTGAGTCATCATCTAGTCCTGAAGTGGAAGAAGCACGGACGCTC
AGAGTCCTCTCCATGGTGGGGGATTGGCTTGAAAAAAGACCAGGAGCCTTTTGTATAAAAGTGTTGTG
CCCATACACCAGCACTATGATGGAAACCCTGGAGCGACTGCAGCGTAGGTATGGGGGAGGACTGGTCA
GAGTGCCACTCTCCCGCAACTCTACACATGAGATGTACTGGGTCTCTGGAGCGAAAAGCAACACCATA
AAAAGTGTGTCCACCACGAGCCAGCTCCTCTTGGGGCGCATGGACGGGCCTAGAAGGCCAGTGAAATA
TGAGGAGGATGTGAATCTCGGCTCTGGCACGCGGGCTGTGGTAAGCTGCGCTGAAGCTCCCAACATGA
AGATCATTGGTAACCGCATTGAAAGGATCCGCAGTGAGCACGCGGAAACGTGGTTCTTTGACGAGAAC
CACCCATATAGGACATGGGCTTACCATGGAAGCTATGAGGCCCCCACACAAGGGTCAGCGTCCTCTCT
AATAAACGGGGTTGTCAGGCTCCTGTCAAAACCCTGGGATGTGGTGACTGGAGTCACAGGAATAGCCA
TGACCGACACCACACCGTATGGTCAGCAAAGAGTTTTCAAGGAAAAAGTGGACACTAGGGTGCCAGAC
CCCCAAGAAGGCACTCGTCAGGTTATGAGCATGGTCTCTTCCTGGTTGTGGAAAGAGCTAGGCAAACA
CAAACGGCCACGAGTCTGTACCAAAGAAGAGTTCATCAACAAGGTTCGTAGCAATGCAGCATTAGGGG
CAATATTTGAAGAGGAAAAAGAGTGGAAGACTGCAGTGGAAGCTGTGAACGATCCAAGGTTCTGGGCT
CTAGTGGACAAGGAAAGAGAGCACCACCTGAGAGGAGAGTGCCAGAGTTGTGTGTATAACATGATGGG
AAAAAGAGAAAAGAAACAAGGGGAATTTGGAAAGGCCAAGGGCAGCCGCGCCATCTGGTATATGTGGC
TAGGGGCTAGATTTCTAGAGTTCGAAGCCCTTGGATTCTTGAACGAGGATCACTGGATGGGGAGAGAG
AACTCAGGAGGTGGTGTTGAAGGGCTGGGATTACAAAGACTCGGATATGTCCTAGAAGAGATGAGTCG
TATACCAGGAGGAAGGATGTATGCAGATGACACTGCTGGCTGGGACACCCGCATTAGCAGGTTTGATC
TGGAGAATGAAGCTCTAATCACCAACCAAATGGAGAAAGGGCACAGGGCCTTGGCATTGGCCATAATC
AAGTACACATACCAAAACAAAGTGGTAAAGGTCCTTAGACCAGCTGAAAAAGGGAAAACAGTTATGGA
CATTATTTCGAGACAAGACCAAAGGGGGAGCGGACAAGTTGTCACTTACGCTCTTAACACATTTACCA
ACCTAGTGGTGCAACTCATTCGGAATATGGAGGCTGAGGAAGTTCTAGAGATGCAAGACTTGTGGCTG
CTGCGGAGGTCAGAGAAAGTGACCAACTGGTTGCAGAGCAACGGATGGGATAGGCTCAAACGAATGGC
AGTCAGTGGAGATGATTGCGTTGTGAAGCCAATTGATGATAGGTTTGCACATGCCCTCAGGTTCTTGA
ATGATATGGGAAAAGTTAGAAAGGACACACAAGAGTGGAAACCCTCAACTGGATGGGACAACTGGGAA
GAAGTTCCGTTTTGCTCCCACCACTTCAACAAGCTCCATCTCAAGGACGGGAGGTCCATTGTGGTTCC
CTGCCGCCACCAAGATGAACTGATTGGCCGGGCCCGCGTCTCTCCAGGGGCGGGATGGAGCATCCGGG
AGACTGCTTGCCTAGCAAAATCATATGCGCAGATGTGGCAGCTCCTTTATTTCCACAGAAGGGACCTC
CGACTGATGGCCAATGCCATTTGTTCATCTGTGCCAGTTGACTGGGTTCCAACTGGGAGAACTACCTG
GTCAATCCATGGAAAGGGAGAATGGATGACCACTGAAGACATGCTTGTGGTGTGGAACAGAGTGTGGA
TTGAGGAGAACGACCACATGGAAGACAAGACCCCAGTTACGAAATGGACAGACATTCCCTATTTGGGA
AAAAGGGAAGACTTGTGGTGTGGATCTCTCATAGGGCACAGACCGCGCACCACCTGGGCTGAGAACAT
TAAAAACACAGTCAACATGGTGCGCAGGATCATAGGTGATGAAGAAAAGTACATGGACTACCTATCCA
CCCAAGTTCGCTACTTGGGTGAAGAAGGGTCTACACCTGGAGTGCTGTAA(NS5 end)

SEQ ID NO:3. Vaccine candidate ZIKV-DO-NS3 nonstructural region nucleotide sequence, showing the codon deoptimized NS3 region. The NS3 region of vaccine candidate ZIKV-DO-NS3 has the same nucleotide changes as the NS3 region of vaccine candidate ZIKV-DO. In the deoptimized region changed nucleotides are marked in bold and underline.

(NS3)AGTGGTGCGCTCTGGGATGTCCCCGCGCCCAAGGAAGTAAAAAAGGGTGAGACCACGGATGGC
GTCTACCGAGTAATGACCCGTCGACTACTCGGTTCGACGCAAGTAGGCGTCGGCGTAATGCAAGAGGG
TGTATTCCACACCATGTGGCATGTAACGAAAGGCTCGGCGCTACGATCCGGTGAAGGTCGATTGGATC
CGTACTGGGGCGATGTAAAGCAAGATCTAGTCTCGTACTGTGGTCCGTGGAAGCTCGATGCCGCCTGG
GACGGTCACTCCGAGGTCCAGTTATTGGCCGTCCCCCCGGGCGAGCGAGCGCGCAATATACAAACTCT
ACCCGGCATATTCAAGACGAAGGATGGTGACATTGGCGCGGTAGCGCTAGATTACCCGGCGGGCACTT
CGGGCTCGCCGATACTCGACAAGTGTGGTCGAGTCATAGGCTTGTATGGTAATGGTGTAGTCATAAAA
AATGGTAGTTATGTAAGTGCCATAACCCAAGGTCGCCGCGAAGAAGAGACCCCCGTAGAGTGCTTCGA
GCCCTCGATGCTAAAGAAGAAGCAACTCACTGTATTAGACTTGCATCCCGGCGCGGGTAAAACCCGCC
GAGTATTGCCCGAAATAGTACGTGAAGCCATAAAAACGCGATTACGTACCGTCATATTAGCGCCGACC
CGCGTAGTAGCGGCGGAAATGGAGGAGGCCTTGCGAGGTTTGCCGGTCCGTTATATGACGACGGCGGT
AAATGTAACCCATTCGGGCACGGAAATAGTAGACTTAATGTGCCATGCCACCTTCACCTCGCGTCTCC
TCCAGCCGATACGAGTACCCAATTATAATCTATATATTATGGATGAGGCCCATTTCACGGATCCCTCG
AGTATAGCGGCGCGAGGCTACATTTCGACGCGCGTAGAGATGGGTGAGGCGGCGGCCATATTCATGAC
CGCCACGCCGCCGGGCACCCGTGACGCGTTCCCGGACTCGAATTCGCCGATTATGGACACCGAAGTCG
AAGTACCGGAGCGAGCCTGGTCCTCGGGTTTTGATTGGGTCACGGATCATTCGGGCAAAACGGTATGG
TTCGTACCGTCCGTCCGCAATGGTAATGAGATAGCGGCGTGTCTAACGAAGGCGGGCAAACGGGTAAT
ACAGTTATCCCGAAAGACCTTCGAGACGGAGTTCCAAAAAACGAAACATCAAGAGTGGGACTTCGTAG
TCACGACCGACATTTCGGAGATGGGTGCCAATTTCAAAGCGGACCGTGTAATAGATTCGCGCCGATGC
CTCAAGCCGGTAATATTGGATGGTGAGCGAGTAATTCTAGCGGGCCCCATGCCCGTAACGCATGCCTC
CGCGGCCCAACGCCGCGGTCGCATAGGTCGCAATCCCAATAAACCCGGCGATGAGTATCTATATGGCG
GTGGTTGCGCGGAGACCGACGAAGACCATGCGCATTGGTTGGAAGCGCGAATGTTATTGGACAATATT
TACTTACAAGATGGTTTAATAGCCTCGTTATATCGACCCGAGGCCGACAAAGTAGCGGCCATTGAGGG
CGAGTTCAAGTTGCGCACGGAGCAACGCAAGACCTTCGTCGAATTAATGAAACGAGGCGATTTGCCCG
TATGGCTAGCCTATCAAGTAGCGTCGGCCGGCATAACCTACACGGATCGACGATGGTGCTTCGATGGT
ACGACCAATAATACCATAATGGAAGATAGTGTGCCGGCAGAGGTGTGGACCAGACACGGAGAGAAAAG
AGTGCTCAAACCGAGGTGGATGGACGCCAGAGTTTGTTCAGATCATGCGGCCCTGAAGTCATTCAAGG
AGTTTGCCGCTGGGAAAAGA (NS3 end)

SEQ ID NO:4. Vaccine candidate ZIKV-DO-NS3 nonstructural region nucleotide sequence, showing the entire codon deoptimized NS3 region, with deoptimized region shown in underline.

(NS3)AGTGGTGCGCTCTGGGATGTCCCCGCGCCCAAGGAAGTAAAAAAGGGTGAGACC
ACGGATGGCGTCTACCGAGTAATGACCCGTCGACTACTCGGTTCGACGCAAGTAGGCGTCGGCGTAAT
GCAAGAGGGTGTATTCCACACCATGTGGCATGTAACGAAAGGCTCGGCGCTACGATCCGGTGAAGGTC
GATTGGATCCGTACTGGGGCGATGTAAAGCAAGATCTAGTCTCGTACTGTGGTCCGTGGAAGCTCGAT
GCCGCCTGGGACGGTCACTCCGAGGTCCAGTTATTGGCCGTCCCGCCGGGCGAGCGAGCGCGCAATAT
ACAAACTCTACCCGGCATATTCAAGACGAAGGATGGTGACATTGGCGCGGTAGCGCTAGATTACCCGG
CGGGCACTTCGGGCTCGCCGATACTCGACAAGTGTGGTCGAGTCATAGGCTTGTATGGTAATGGTGTA
GTCATAAAAAATGGTAGTTATGTAAGTGCCATAACCCAAGGTCGCCGCGAAGAAGAGACCCCCGTAGA
GTGCTTCGAGCCCTCGATGCTAAAGAAGAAGCAACTCACTGTATTAGACTTGCATCCCGGCGCGGGTA
AAACCCGCCGAGTATTGCCCGAAATAGTACGTGAAGCCATAAAAACGCGATTACGTACCGTCATATTA
GCGCCGACCCGCGTAGTAGCGGCGGAAATGGAGGAGGCCTTGCGAGGTTTGCCAGTCCGTTATATGAC
GACGGCGGTAAATGTAACCCATTCGGGCACGGAAATAGTAGACTTAATGTGCCATGCCACCTTCACCT
CGCGTCTCCTCCAGCCGATACGAGTACCCAATTATAATCTATATATTATGGATGAGGCCCATTTCACG
GATCCCTCGAGTATAGCGGCGCGAGGCTACATTTCGACGCGCGTAGAGATGGGTGAGGCGGCGGCCAT
ATTCATGACCGCCACGCCGCCGGGCACCCGTGACGCGTTCCCGGACTCGAATTCGCCGATTATGGACA
CCGAAGTCGAAGTACCGGAGCGAGCCTGGTCCTCGGGTTTTGATTGGGTCACGGATCATTCGGGCAAA
ACGGTATGGTTCGTACCGTCCGTCCGCAATGGTAATGAGATAGCGGCGTGTCTAACGAAGGCGGGCAA
ACGGGTAATACAGTTATCCCGAAAGACCTTCGAGACGGAGTTCCAAAAAACGAAACATCAAGAGTGGG
ACTTCGTAGTCACGACCGACATTTCGGAGATGGGTGCCAATTTCAAAGCAGACCGTGTAATAGATTCG
CGCCGATGCCTCAAGCCGGTAATATTGGATGGTGAGCGAGTAATTCTAGCGGGCCCCATGCCCGTAAC
GCATGCCTCCGCGGCCCAACGCCGCGGTCGCATAGGTCGCAATCCCAATAAACCCGGCGATGAGTATC
TATATGGCGGTGGTTGCGCGGAGACCGACGAAGACCATGCGCATTGGTTGGAAGCGCGAATGTTATTG
GACAATATTTACTTACAAGATGGTTTAATAGCCTCGTTATATCGACCCGAGGCCGACAAAGTAGCGGC
CATTGAGGGCGAGTTCAAGTTGCGCACGGAGCAACGCAAGACCTTCGTCGAATTAATGAAACGAGGCG
ATTTGCCCGTATGGCTAGCCTATCAAGTAGCGTCGGCAGGTATAACCTACACGGATCGACGATGGTGC
TTCGATGGTACGACCAATAATACCATAATGGAAGATAGTGTGCCGGCAGAGGTGTGGACCAGACACGG
AGAGAAAAGAGTGCTCAAACCGAGGTGGATGGACGCCAGAGTTTGTTCAGATCATGCGGCCCTGAAGT
CATTCAAGGAGTTTGCCGCTGGGAAAAGA

SEQ ID NO:5. Vaccine candidate ZIKV-DO-NS3, with deoptimized region shown in underline, with locations of nonstructural regions indicated. The entire NS3 region of vaccine candidate ZIKV-DO-NS3 is shown together with all flanking nonstructural regions (NS1 to NS5).

(NS1)GATGTGGGGTGCTCGGTGGACTTCTCAAAGAAGGAGACGAGATGCGGTACAGGG
GTGTTCGTCTATAACGACGTTGAAGCCTGGAGGGACAGGTACAAGTACCATCCTGACTCCCCCCGTAG
ATTGGCAGCAGCAGTCAAGCAAGCCTGGGAAGATGGTATCTGCGGGATCTCCTCTGTTTCAAGAATGG
AAAACATCATGTGGAGATCAGTAGAAGGGGAGCTCAACGCAATCCTGGAAGAGAATGGAGTTCAACTG
ACGGTCGTTGTGGGATCTGTAAAAAACCCCATGTGGAGAGGTCCACAGAGATTGCCCGTGCCTGTGAA
CGAGCTGCCCCACGGCTGGAAGGCTTGGGGGAAATCGTACTTCGTCAGAGCAGCAAAGACAAATAACA
GCTTTGTCGTGGATGGTGACACACTGAAGGAATGCCCACTCAAACATAGAGCATGGAACAGCTTTCTT
GTGGAGGATCATGGGTTCGGGGTATTTCACACTAGTGTCTGGCTCAAGGTTAGAGAAGATTATTCATT
AGAGTGTGATCCAGCCGTTATTGGAACAGCTGTTAAGGGAAAGGAGGCTGTACACAGTGATCTAGGCT
ACTGGATTGAGAGTGAGAAGAATGACACATGGAGGCTGAAGAGGGCCCATCTGATCGAGATGAAAACA
TGTGAATGGCCAAAGTCCCACACATTGTGGACAGATGGAATAGAAGAGAGTGATCTGATCATACCCAA
GTCTTTAGCTGGGCCACTCAGCCATCACAATACCAGAGAGGGCTACAGGACCCAAATGAAAGGGCCAT
GGCACAGTGAAGAGCTTGAAATTCGGTTTGAGGAATGCCCAGGCACTAAGGTCCACGTGGAGGAAACA
TGTGGAACAAGAGGACCATCTCTGAGATCAACCACTGCAAGCGGAAGGGTGATCGAGGAATGGTGCTG
CAGGGAGTGCACAATGCCCCCACTGTCGTTCCGGGCTAAAGATGGCTGTTGGTATGGAATGGAGATAA
GGCCCAGGAAAGAACCAGAAAGCAACTTAGTAAGGTCAATGGTGACTGCA(NS2A)GGATCAACTGAT
CACATGGACCACTTCTCCCTTGGAGTGCTTGTGATCCTGCTCATGGTGCAGGAAGGGCTGAAGAAGAG
AATGACCACAAAGATCATCATAAGCACATCAATGGCAGTGCTGGTAGCTATGATCCTGGGAGGATTTT
CAATGAGTGACCTGGCTAAGCTTGCAATTTTGATGGGTGCCACCTTCGCGGAAATGAACACTGGAGGA
GATGTAGCTCATCTGGCGCTGATAGCGGCATTCAAAGTCAGACCAGCGTTGCTGGTATCTTTCATCTT
CAGAGCTAATTGGACACCCCGTGAAAGCATGCTGCTGGCCTTGGCCTCGTGTCTTTTGCAAACTGCGA
TCTCCGCCTTGGAAGGCGACCTGATGGTTCTCATCAATGGTTTTGCTTTGGCCTGGTTGGCAATACGA
GCGATGGTTGTTCCACGCACTGATAACATCACCTTGGCAATCCTGGCTGCTCTGACACCACTGGCCCG
GGGCACACTGCTTGTGGCGTGGAGAGCAGGCCTTGCTACTTGCGGGGGGTTTATGCTCCTCTCTCTGA
AGGGAAAAGGCAGTGTGAAGAAGAACTTACCATTTGTCATGGCCCTGGGACTAACCGCTGTGAGGCTG
GTCGACCCCATCAACGTGGTGGGACTGCTGTTACTCACAAGGAGTGGGAAGCGG(NS2B)AGCTGGCC
CCCTAGCGAAGTACTCACAGCTGTTGGCCTGATATGCGCATTGGCTGGAGGGTTCGCCAAGGCAGATA
TAGAGATGGCTGGGCCCATGGCCGCGGTCGGTCTGCTAATTGTCAGTTACGTGGTCTCAGGAAAGAGT
GTGGACATGTACATTGAAAGAGCAGGTGACATCACATGGGAAAAAGATGCGGAAGTCACTGGAAACAG
TCCCCGGCTCGATGTGGCGCTAGATGAGAGTGGTGATTTCTCCCTGGTGGAGGATGACGGTCCCCCCA
TGAGAGAGATCATACTCAAGGTGGTCCTGATGACCATCTGTGGCATGAATCCAATAGCCATACCCTTT
GCAGCTGGAGCGTGGTACGTATACGTGAAGACTGGAAAAAGG(NS3)AGTGGTGCGCTCTGGGATGTC
CCCGCGCCCAAGGAAGTAAAAAAGGGTGAGACCACGGATGGCGTCTACCGAGTAATGACCCGTCGACT
ACTCGGTTCGACGCAAGTAGGCGTCGGCGTAATGCAAGAGGGTGTATTCCACACCATGTGGCATGTAA
CGAAAGGCTCGGCGCTACGATCCGGTGAAGGTCGATTGGATCCGTACTGGGGCGATGTAAAGCAAGAT
CTAGTCTCGTACTGTGGTCCGTGGAAGCTCGATGCCGCCTGGGACGGTCACTCCGAGGTCCAGTTATT
GGCCGTCCCGCCGGGCGAGCGAGCGCGCAATATACAAACTCTACCCGGCATATTCAAGACGAAGGATG
GTGACATTGGCGCGGTAGCGCTAGATTACCCGGCGGGCACTTCGGGCTCGCCGATACTCGACAAGTGT
GGTCGAGTCATAGGCTTGTATGGTAATGGTGTAGTCATAAAAAATGGTAGTTATGTAAGTGCCATAAC
CCAAGGTCGCCGCGAAGAAGAGACCCCCGTAGAGTGCTTCGAGCCCTCGATGCTAAAGAAGAAGCAAC
TCACTGTATTAGACTTGCATCCCGGCGCGGGTAAAACCCGCCGAGTATTGCCCGAAATAGTACGTGAA
GCCATAAAAACGCGATTACGTACCGTCATATTAGCGCCGACCCGCGTAGTAGCGGCGGAAATGGAGGA
GGCCTTGCGAGGTTTGCCAGTCCGTTATATGACGACGGCGGTAAATGTAACCCATTCGGGCACGGAAA
TAGTAGACTTAATGTGCCATGCCACCTTCACCTCGCGTCTCCTCCAGCCGATACGAGTACCCAATTAT
AATCTATATATTATGGATGAGGCCCATTTCACGGATCCCTCGAGTATAGCGGCGCGAGGCTACATTTC
GACGCGCGTAGAGATGGGTGAGGCGGCGGCCATATTCATGACCGCCACGCCGCCGGGCACCCGTGACG
CGTTCCCGGACTCGAATTCGCCGATTATGGACACCGAAGTCGAAGTACCGGAGCGAGCCTGGTCCTCG
GGTTTTGATTGGGTCACGGATCATTCGGGCAAAACGGTATGGTTCGTACCGTCCGTCCGCAATGGTAA
TGAGATAGCGGCGTGTCTAACGAAGGCGGGCAAACGGGTAATACAGTTATCCCGAAAGACCTTCGAGA
CGGAGTTCCAAAAAACGAAACATCAAGAGTGGGACTTCGTAGTCACGACCGACATTTCGGAGATGGGT
GCCAATTTCAAAGCAGACCGTGTAATAGATTCGCGCCGATGCCTCAAGCCGGTAATATTGGATGGTGA
GCGAGTAATTCTAGCGGGCCCCATGCCCGTAACGCATGCCTCCGCGGCCCAACGCCGCGGTCGCATAG
GTCGCAATCCCAATAAACCCGGCGATGAGTATCTATATGGCGGTGGTTGCGCGGAGACCGACGAAGAC
CATGCGCATTGGTTGGAAGCGCGAATGTTATTGGACAATATTTACTTACAAGATGGTTTAATAGCCTC
GTTATATCGACCCGAGGCCGACAAAGTAGCGGCCATTGAGGGCGAGTTCAAGTTGCGCACGGAGCAAC
GCAAGACCTTCGTCGAATTAATGAAACGAGGCGATTTGCCCGTATGGCTAGCCTATCAAGTAGCGTCG
GCAGGTATAACCTACACGGATCGACGATGGTGCTTCGATGGTACGACCAATAATACCATAATGGAAGA
TAGTGTGCCGGCAGAGGTGTGGACCAGACACGGAGAGAAAAGAGTGCTCAAACCGAGGTGGATGGACG
CCAGAGTTTGTTCAGATCATGCGGCCCTGAAGTCATTCAAGGAGTTTGCCGCTGGGAAAAGA(NS4A)
GGAGCGGCTTTTGGAGTGATGGAAGCCCTGGGAACACTGCCAGGACACATGACAGAGAGATTCCAGGA
AGCCATTGACAACCTCGCTGTGCTCATGCGGGCAGAGACTGGAAGCAGGCCTTACAAAGCCGCGGCGG
CCCAATTGCCGGAGACCCTAGAGACCATAATGCTTTTGGGGTTGCTGGGAACAGTCTCGCTGGGAATC
TTCTTCGTCTTGATGAGGAACAAGGGCATAGGGAAGATGGGCTTTGGAATGGTGACTCTTGGGGCCAG
CGCATGGCTCATGTGGCTCTCGGAAATTGAGCCAGCCAGAATTGCATGTGTCCTCATTGTTGTGTTCC
TATTGCTGGTGGTGCTCATACCTGAGCCAGAAAAGCAAAGATCTCCCCAGGACAACCAAATGGCAATC
ATCATCATGGTAGCAGTAGGTCTTCTGGGCTTGATTACCGCC(NS4B)AATGAACTCGGATGGTTGGA
GAGAACAAAGAGTGACCTAAGCCATCTAATGGGAAGGAGAGAGGAGGGGGCAACCATAGGATTCTCAA
TGGACATTGACCTGCGGCCAGCCTCAGCTTGGGCCATCTATGCTGCCTTGACAACTTTCATTACCCCA
GCCGTCCAACATGCAGTGACCACCTCATACAACAACTACTCCTTAATGGCGATGGCCACGCAAGCTGG
AGTGTTGTTTGGTATGGGCAAAGGGATGCCATTCTACGCATGGGACTTTGGAGTCCCGCTGCTAATGA
TAGGTTGCTACTCACAATTAACACCCCTGACCCTAATAGTGGCCATCATTTTGCTCGTGGCGCACTAC
ATGTACTTGATCCCAGGGCTGCAGGCAGCAGCTGCGCGTGCTGCCCAGAAGAGAACGGCAGCTGGCAT
CATGAAGAACCCTGTTGTGGATGGAATAGTGGTGACTGACATTGACACAATGACAATTGACCCCCAAG
TGGAGAAAAAGATGGGACAGGTGCTACTCATAGCAGTAGCCGTCTCCAGCGCCATACTGTCGCGGACC
GCCTGGGGGTGGGGGGAGGCTGGGGCCCTGATCACAGCCGCAACTTCCACTTTGTGGGAAGGCTCTCC
GAACAAGTACTGGAACTCCTCTACAGCCACTTCACTGTGTAACATTTTTAGGGGAAGTTACTTGGCTG
GAGCTTCTCTAATCTACACAGTAACAAGAAACGCTGGCTTGGTCAAGAGACGT(NS5)GGGGGTGGAA
CAGGAGAGACCCTGGGAGAGAAATGGAAGGCCCGCTTGAACCAGATGTCGGCCCTGGAGTTCTACTCC
TACAAAAAGTCAGGCATCACCGAGGTGTGCAGAGAAGAGGCCCGCCGCGCCCTCAAGGACGGTGTGGC
AACGGGAGGCCATGCTGTGTCCCGAGGAAGTGCAAAGCTGAGATGGTTGGTGGAGCGGGGATACCTGC
AGCCCTATGGAAAGGTCATTGATCTTGGATGTGGCAGAGGGGGCTGGAGTTACTACGCCGCCACCATC
CGCAAAGTTCAAGAAGTGAAAGGATACACAAAAGGAGGCCCTGGTCATGAAGAACCCGTGTTGGTGCA
AAGCTATGGGTGGAACATAGTCCGTCTTAAGAGTGGGGTGGACGTCTTTCATATGGCGGCTGAGCCGT
GTGACACGCTGCTGTGTGACATAGGTGAGTCATCATCTAGTCCTGAAGTGGAAGAAGCACGGACGCTC
AGAGTCCTCTCCATGGTGGGGGATTGGCTTGAAAAAAGACCAGGAGCCTTTTGTATAAAAGTGTTGTG
CCCATACACCAGCACTATGATGGAAACCCTGGAGCGACTGCAGCGTAGGTATGGGGGAGGACTGGTCA
GAGTGCCACTCTCCCGCAACTCTACACATGAGATGTACTGGGTCTCTGGAGCGAAAAGCAACACCATA
AAAAGTGTGTCCACCACGAGCCAGCTCCTCTTGGGGCGCATGGACGGGCCTAGAAGGCCAGTGAAATA
TGAGGAGGATGTGAATCTCGGCTCTGGCACGCGGGCTGTGGTAAGCTGCGCTGAAGCTCCCAACATGA
AGATCATTGGTAACCGCATTGAAAGGATCCGCAGTGAGCACGCGGAAACGTGGTTCTTTGACGAGAAC
CACCCATATAGGACATGGGCTTACCATGGAAGCTATGAGGCCCCCACACAAGGGTCAGCGTCCTCTCT
AATAAACGGGGTTGTCAGGCTCCTGTCAAAACCCTGGGATGTGGTGACTGGAGTCACAGGAATAGCCA
TGACCGACACCACACCGTATGGTCAGCAAAGAGTTTTCAAGGAAAAAGTGGACACTAGGGTGCCAGAC
CCCCAAGAAGGCACTCGTCAGGTTATGAGCATGGTCTCTTCCTGGTTGTGGAAAGAGCTAGGCAAACA
CAAACGGCCACGAGTCTGTACCAAAGAAGAGTTCATCAACAAGGTTCGTAGCAATGCAGCATTAGGGG
CAATATTTGAAGAGGAAAAAGAGTGGAAGACTGCAGTGGAAGCTGTGAACGATCCAAGGTTCTGGGCT
CTAGTGGACAAGGAAAGAGAGCACCACCTGAGAGGAGAGTGCCAGAGTTGTGTGTATAACATGATGGG
AAAAAGAGAAAAGAAACAAGGGGAATTTGGAAAGGCCAAGGGCAGCCGCGCCATCTGGTATATGTGGC
TAGGGGCTAGATTTCTAGAGTTCGAAGCCCTTGGATTCTTGAACGAGGATCACTGGATGGGGAGAGAG
AACTCAGGAGGTGGTGTTGAAGGGCTGGGATTACAAAGACTCGGATATGTCCTAGAAGAGATGAGTCG
TATACCAGGAGGAAGGATGTATGCAGATGACACTGCTGGCTGGGACACCCGCATTAGCAGGTTTGATC
TGGAGAATGAAGCTCTAATCACCAACCAAATGGAGAAAGGGCACAGGGCCTTGGCATTGGCCATAATC
AAGTACACATACCAAAACAAAGTGGTAAAGGTCCTTAGACCAGCTGAAAAAGGGAAAACAGTTATGGA
CATTATTTCGAGACAAGACCAAAGGGGGAGCGGACAAGTTGTCACTTACGCTCTTAACACATTTACCA
ACCTAGTGGTGCAACTCATTCGGAATATGGAGGCTGAGGAAGTTCTAGAGATGCAAGACTTGTGGCTG
CTGCGGAGGTCAGAGAAAGTGACCAACTGGTTGCAGAGCAACGGATGGGATAGGCTCAAACGAATGGC
AGTCAGTGGAGATGATTGCGTTGTGAAGCCAATTGATGATAGGTTTGCACATGCCCTCAGGTTCTTGA
ATGATATGGGAAAAGTTAGAAAGGACACACAAGAGTGGAAACCCTCAACTGGATGGGACAACTGGGAA
GAAGTTCCGTTTTGCTCCCACCACTTCAACAAGCTCCATCTCAAGGACGGGAGGTCCATTGTGGTTCC
CTGCCGCCACCAAGATGAACTGATTGGCCGGGCCCGCGTCTCTCCAGGGGCGGGATGGAGCATCCGGG
AGACTGCTTGCCTAGCAAAATCATATGCGCAGATGTGGCAGCTCCTTTATTTCCACAGAAGGGACCTC
CGACTGATGGCCAATGCCATTTGTTCATCTGTGCCAGTTGACTGGGTTCCAACTGGGAGAACTACCTG
GTCAATCCATGGAAAGGGAGAATGGATGACCACTGAAGACATGCTTGTGGTGTGGAACAGAGTGTGGA
TTGAGGAGAACGACCACATGGAAGACAAGACCCCAGTTACGAAATGGACAGACATTCCCTATTTGGGA
AAAAGGGAAGACTTGTGGTGTGGATCTCTCATAGGGCACAGACCGCGCACCACCTGGGCTGAGAACAT
TAAAAACACAGTCAACATGGTGCGCAGGATCATAGGTGATGAAGAAAAGTACATGGACTACCTATCCA
CCCAAGTTCGCTACTTGGGTGAAGAAGGGTCTACACCTGGAGTGCTGTAA(NS5 end)

SEQ ID NO:6. Vaccine candidate ZIKV-DO-scattered entire nonstructural region nucleotide sequence, with locations of nonstructural regions indicated. In the deoptimized region changed nucleotides are marked in bold and underline.

(NS1)GATGTAGGGTGCTCGGTAGACTTCTCAAAGAAGGAAACGAGATGCGGTACGGGG
GTATTCGTCTATAACGATGTTGAAGCCTGGCGTGACAGGTACAAATACCATCCTGATTCCCCCCGTCG
ATTGGCAGCAGCGGTCAAGCAAGCGTGGGAAGATGGTATATGCGGGATCTCGTCTGTTTCACGTATGG
AAAACATAATGTGGAGATCGGTAGAAGGGGAGCTAAACGCAATCCTAGAAGAGAATGGTGTTCAACTG
ACGGTAGTTGTAGGATCTGTAAAAAACCCGATGTGGAGAGGTCCGCAGAGATTGCCGGTACCTGTAAA
CGAGCTGCCCCACGGTTGGAAGGCTTGGGGTAAATCGTACTTCGTAAGAGCAGCAAAAACAAATAACT
CGTTTGTCGTGGATGGTGATACACTGAAGGAATGTCCACTCAAACATCGTGCATGGAACTCGTTTCTT
GTAGAGGATCATGGTTTCGGGGTATTTCATACTAGTGTCTGGCTAAAGGTTAGAGAAGATTATTCGTT
AGAGTGTGATCCGGCCGTTATTGGTACAGCTGTTAAAGGAAAGGAGGCGGTACACAGTGATCTAGGTT
ACTGGATTGAAAGTGAGAAGAATGATACATGGAGGCTAAAGAGGGCCCATCTAATCGAGATGAAAACG
TGTGAATGGCCGAAGTCCCACACGTTGTGGACAGATGGTATAGAAGAGTCGGATCTGATCATACCGAA
GTCTTTAGCGGGGCCACTCAGTCATCACAATACGAGAGAGGGCTATAGGACCCAAATGAAAGGTCCAT
GGCACTCGGAAGAGCTTGAAATACGGTTTGAGGAATGTCCAGGCACTAAAGTCCACGTGGAAGAAACA
TGTGGTACAAGAGGACCGTCTCTGAGATCGACCACTGCAAGTGGAAGGGTAATCGAGGAATGGTGTTG
CAGGGAGTGCACGATGCCCCCACTATCGTTCCGGGCGAAAGATGGCTGTTGGTATGGTATGGAGATAC
GTCCCAGGAAAGAACCGGAAAGCAACTTAGTACGTTCAATGGTAACTGCA(NS2A)GGATCGACTGAT
CACATGGATCACTTCTCCCTTGGAGTACTTGTAATCCTGCTCATGGTACAGGAAGGGCTAAAGAAGAG
AATGACGACAAAGATCATAATAAGCACATCGATGGCAGTACTGGTAGCTATGATACTGGGAGGATTTT
CGATGAGTGACCTAGCTAAGCTTGCGATTTTGATGGGTGCGACCTTCGCGGAAATGAATACTGGAGGA
GATGTAGCGCATCTGGCGCTAATAGCGGCATTTAAAGTCAGACCGGCGTTGCTGGTATCGTTCATCTT
CCGUGCTAATTGGACGCCCCGTGAATCGATGCTGCTGGCGTTGGCCTCGTGTCTATTGCAAACTGCGA
TATCCGCCTTGGAAGGTGACCTGATGGTACTCATCAATGGTTTTGCGTTGGCCTGGTTAGCAATACGA
GCGATGGTAGTTCCACGCACGGATAACATCACGTTGGCAATCCTAGCTGCTCTGACGCCACTGGCCCG
TGGCACACTGCTTGTAGCGTGGAGAGCGGGCCTTGCTACGTGCGGGGGGTTTATGCTACTCTCTCTGA
AAGGAAAAGGCAGTGTAAAGAAGAACTTACCGTTTGTCATGGCGCTGGGACTAACGGCTGTAAGGCTG
GTCGATCCCATCAACGTAGTAGGACTGCTGTTACTAACAAGGAGTGGGAAACGG(NS2B)AGCTGGCC
GCCTAGCGAAGTACTAACAGCTGTTGGTCTGATATGCGCATTGGCGGGAGGGTTCGCGAAGGCAGATA
TAGAAATGGCTGGGCCGATGGCCGCGGTAGGTCTGCTAATAGTCAGTTACGTAGTCTCAGGAAAAAGT
GTGGACATGTATATTGAAAGAGCGGGTGACATCACATGGGAAAAAGATGCGGAAGTAACTGGAAACAG
TCCGCGGCTCGATGTAGCGCTAGATGAAAGTGGTGATTTTTCCCTGGTAGAGGATGACGGTCCCCCCA
TGAGAGAAATCATACTCAAAGTAGTCCTGATGACGATCTGTGGCATGAATCCGATAGCCATACCGTTT
GCAGCTGGTGCGTGGTACGTATACGTAAAGACTGGAAAACGT(NS3)AGTGGTGCTCTATGGGATGTA
CCTGCTCCCAAAGAAGTAAAAAAAGGGGAGACCACGGATGGAGTATACAGAGTAATGACGCGTAGACT
GCTAGGTTCGACACAAGTTGGTGTAGGAGTTATGCAAGAAGGGGTCTTTCATACTATGTGGCATGTCA
CAAAAGGTTCCGCGCTGCGTAGCGGTGAAGGTAGACTTGATCCGTACTGGGGAGATGTAAAGCAGGAT
CTAGTATCATACTGTGGTCCGTGGAAGCTAGATGCGGCCTGGGACGGTCACAGCGAGGTACAGCTCTT
GGCGGTACCCCCCGGAGAAAGAGCGAGGAATATCCAGACTCTACCCGGAATATTTAAAACAAAGGATG
GTGACATTGGAGCGGTAGCGCTGGATTATCCAGCAGGAACGTCAGGATCTCCGATCCTAGACAAATGT
GGGAGAGTAATAGGACTTTATGGTAATGGGGTCGTAATCAAAAATGGTAGTTATGTTAGTGCGATCAC
CCAAGGTAGGAGGGAAGAAGAAACTCCTGTTGAATGCTTCGAGCCGTCGATGCTGAAAAAGAAGCAGC
TAACGGTCTTAGACTTACATCCTGGAGCGGGGAAAACCCGAAGAGTTCTTCCGGAAATAGTCCGTGAA
GCGATAAAAACACGTCTCCGTACTGTAATCTTAGCTCCGACCAGGGTTGTAGCTGCTGAAATGGAAGA
GGCCCTTCGTGGGCTTCCAGTACGTTATATGACGACAGCAGTCAATGTAACCCACTCTGGTACAGAAA
TCGTTGACTTAATGTGTCATGCCACCTTTACTTCACGTCTACTACAACCAATCAGAGTTCCCAACTAT
AATCTATATATTATGGATGAAGCCCACTTCACGGATCCCTCAAGTATAGCGGCAAGAGGATATATTTC
AACAAGGGTTGAAATGGGCGAGGCGGCGGCCATCTTCATGACGGCCACGCCACCGGGAACCCGTGATG
CATTTCCGGATTCCAACTCACCGATTATGGACACGGAAGTGGAAGTTCCAGAGAGAGCGTGGAGCTCA
GGTTTTGATTGGGTAACGGATCATTCGGGAAAAACAGTTTGGTTTGTTCCGAGCGTGAGGAATGGCAA
TGAGATAGCAGCTTGTCTAACAAAGGCTGGTAAACGGGTCATACAACTCAGCAGAAAAACTTTTGAAA
CAGAGTTCCAAAAAACAAAACATCAAGAATGGGACTTTGTTGTTACAACTGACATATCAGAGATGGGT
GCCAACTTTAAAGCTGACCGTGTCATAGATTCGAGGAGATGCCTAAAGCCGGTCATACTAGATGGCGA
GCGAGTCATTCTGGCGGGACCCATGCCGGTCACACATGCGAGCGCTGCCCAAAGGAGGGGGCGTATAG
GCAGGAATCCGAACAAACCTGGTGATGAGTATCTATATGGAGGTGGTTGCGCAGAGACGGACGAAGAC
CATGCGCACTGGCTTGAAGCGAGAATGCTCCTAGACAATATTTATCTCCAAGATGGTCTCATAGCCTC
GCTATATCGACCTGAAGCCGACAAAGTAGCGGCCATTGAGGGTGAGTTCAAGCTAAGGACGGAGCAAC
GTAAGACCTTTGTAGAACTCATGAAAAGAGGTGATCTTCCTGTATGGCTGGCCTATCAAGTTGCATCT
GCGGGAATAACCTATACAGATAGAAGATGGTGTTTTGATGGCACGACGAACAACACCATAATGGAAGA
TTCGGTGCCGGCAGAAGTGTGGACCAGACATGGAGAGAAACGTGTGCTCAAACCGAGGTGGATGGATG
CCAGAGTTTGTTCAGATCATGCGGCGCTGAAGTCATTTAAGGAGTTTGCGGCTGGGAAAAGA(NS4A)
GGTGCGGCTTTTGGTGTAATGGAAGCCCTGGGAACACTGCCGGGACACATGACGGAGAGATTCCAAGA
AGCCATTGATAACCTCGCTGTACTCATGCGGGCGGAGACTGGAAGTAGGCCTTACAAAGCCGCGGCGG
CGCAATTGCCGGAAACCCTAGAGACGATAATGCTTTTAGGGTTGCTGGGTACAGTCTCGCTAGGAATC
TTCTTTGTCTTGATGCGTAACAAGGGCATAGGTAAGATGGGCTTTGGTATGGTGACTCTAGGGGCCAG
CGCGTGGCTCATGTGGCTATCGGAAATTGAACCAGCCAGAATAGCATGTGTCCTAATTGTTGTATTCC
TATTGCTGGTAGTACTCATACCTGAACCAGAAAAGCAACGTTCTCCCCAGGATAACCAAATGGCAATA
ATCATCATGGTAGCGGTAGGTCTTCTAGGCTTGATTACGGCCU(NS4B)AATGAACTAGGATGGTTGG
AAAGAACAAAGTCGGACCTAAGCCATCTAATGGGTAGGAGAGAGGAAGGGGCAACCATAGGTTTCTCA
ATGGATATTGACCTGCGTCCAGCCTCAGCGTGGGCCATCTATGCGGCCTTGACAACGTTCATTACCCC
GGCCGTCCAACATGCGGTGACCACCTCGTACAACAACTATTCCTTAATGGCGATGGCGACGCAAGCTG
GTGTGTTGTTTGGTATGGGTAAAGGGATGCCGTTCTACGCATGGGATTTTGGAGTCCCGCTACTAATG
ATAGGTTGTTACTCACAATTAACGCCCCTGACCCTAATAGTAGCCATCATTTTACTCGTGGCGCATTA
CATGTACTTAATCCCAGGGCTACAGGCAGCAGCGGCGCGTGCTGCGCAGAAGAGAACGGCGGCTGGCA
TCATGAAAAACCCTGTTGTAGATGGAATAGTAGTGACTGACATAGACACAATGACGATTGACCCCCAA
GTAGAGAAAAAGATGGGTCAGGTGCTACTAATAGCAGTAGCGGTCTCCAGCGCGATACTGTCGCGGAC
CGCCTGGGGTTGGGGGGAGGCGGGGGCCCTGATAACAGCCGCAACGTCCACTTTGTGGGAAGGTTCTC
CGAACAAATACTGGAACTCGTCTACAGCCACGTCACTGTGTAATATTTTTAGGGGTAGTTACTTGGCG
GGAGCTTCTCTAATATACACAGTAACGAGAAACGCTGGTTTGGTCAAGCGTCGT(NS5)GGGGGTGGT
ACAGGAGAGACGCTGGGAGAGAAATGGAAAGCCCGCTTGAATCAGATGTCGGCGCTGGAGTTCTATTC
CTACAAAAAATCAGGCATCACGGAGGTGTGCCGTGAAGAGGCCCGTCGCGCCCTCAAAGACGGTGTGG
CGACGGGAGGCCATGCGGTGTCCCGAGGTAGTGCAAAGCTAAGATGGTTGGTAGAGCGGGGATATCTG
CAGCCCTATGGTAAGGTCATTGATCTAGGATGTGGCCGTGGGGGCTGGTCGTACTACGCCGCGACCAT
CCGCAAAGTACAAGAAGTGAAAGGTTACACAAAAGGTGGCCCTGGTCATGAAGAACCCGTGTTGGTGC
AAAGTTATGGGTGGAATATAGTCCGTCTAAAGAGTGGGGTAGACGTCTTTCATATGGCGGCGGAGCCG
TGTGATACGCTGCTGTGTGATATAGGTGAGTCGTCATCTAGTCCGGAAGTGGAAGAAGCGCGGACGCT
CCGTGTCCTCTCCATGGTAGGGGATTGGCTAGAAAAAAGACCGGGAGCCTTTTGTATAAAAGTATTGT
GCCCATATACCAGCACTATGATGGAAACGCTGGAGCGACTACAGCGTAGGTATGGTGGAGGACTGGTA
AGAGTGCCACTATCCCGCAACTCGACACATGAGATGTATTGGGTCTCTGGTGCGAAAAGCAATACCAT
AAAAAGTGTATCCACCACGTCGCAGCTCCTCTTAGGGCGCATGGATGGGCCTAGACGTCCAGTGAAAT
ATGAAGAGGATGTGAATCTAGGCTCTGGCACGCGTGCTGTGGTAAGTTGCGCTGAAGCGCCCAACATG
AAAATCATTGGTAATCGCATTGAAAGGATACGCAGTGAGCATGCGGAAACGTGGTTTTTTGACGAGAA
TCACCCATATAGGACGTGGGCTTACCATGGTAGCTATGAGGCGCCCACACAAGGTTCAGCGTCCTCGC
TAATAAACGGTGTTGTCAGGCTACTGTCAAAACCGTGGGATGTGGTAACTGGAGTCACGGGAATAGCC
ATGACGGACACCACACCGTATGGTCAACAAAGAGTTTTTAAGGAAAAAGTAGACACTAGGGTACCAGA
CCCCCAAGAAGGTACTCGTCAGGTAATGAGCATGGTATCTTCCTGGTTATGGAAAGAGCTAGGTAAAC
ACAAACGTCCACGAGTCTGTACGAAAGAAGAGTTTATCAACAAGGTACGTAGCAATGCGGCATTAGGG
GCGATATTTGAAGAAGAAAAAGAGTGGAAAACTGCAGTGGAAGCGGTGAACGATCCGAGGTTCTGGGC
GCTAGTGGACAAAGAAAGAGAGCATCACCTGAGAGGTGAGTGCCAGTCGTGTGTGTATAATATGATGG
GAAAACGTGAAAAGAAACAAGGTGAATTTGGAAAAGCCAAGGGCAGCCGTGCCATCTGGTATATGTGG
CTAGGTGCTAGATTTCTAGAATTCGAAGCCCTTGGATTCTTGAATGAGGATCACTGGATGGGTAGAGA
GAACTCGGGAGGTGGTGTAGAAGGGCTGGGTTTACAAAGACTAGGATATGTCCTAGAAGAAATGAGTC
GTATACCGGGAGGAAGGATGTATGCGGATGACACTGCGGGCTGGGACACGCGCATTAGCCGTTTTGAT
CTGGAAAATGAAGCTCTAATAACCAACCAAATGGAAAAAGGGCACAGGGCGTTGGCATTGGCGATAAT
CAAGTATACATACCAAAATAAAGTGGTAAAAGTCCTTAGACCGGCTGAAAAAGGTAAAACAGTTATGG
ATATTATTTCGCGTCAAGACCAAAGGGGTAGCGGACAAGTAGTCACTTACGCGCTTAACACATTTACG
AACCTAGTGGTACAACTCATTCGTAATATGGAGGCGGAGGAAGTTCTAGAAATGCAAGACTTATGGCT
GCTGCGTAGGTCAGAGAAAGTAACCAACTGGTTACAGAGCAACGGTTGGGATAGGCTAAAACGAATGG
CGGTCAGTGGAGATGATTGTGTTGTGAAGCCGATTGATGATCGTTTTGCACATGCGCTCAGGTTCTTA
AATGATATGGGTAAAGTTAGAAAAGACACACAAGAATGGAAACCCTCGACTGGATGGGATAACTGGGA
AGAAGTACCGTTTTGCTCGCACCACTTCAATAAGCTCCATCTAAAGGACGGGCGTTCCATTGTGGTAC
CCTGCCGCCATCAAGATGAACTAATTGGCCGGGCGCGCGTCTCTCCGGGGGCGGGATGGTCGATCCGG
GAGACGGCTTGCCTAGCGAAATCATATGCGCAAATGTGGCAGCTACTTTATTTCCATAGAAGGGACCT
ACGACTGATGGCGAATGCCATTTGTTCGTCTGTGCCAGTAGACTGGGTTCCGACTGGGAGAACGACCT
GGTCAATACATGGAAAGGGTGAATGGATGACGACTGAAGACATGCTAGTGGTGTGGAATAGAGTGTGG
ATAGAGGAGAACGATCACATGGAAGATAAGACCCCAGTAACGAAATGGACGGACATTCCCTATTTAGG
AAAAAGGGAAGATTTGTGGTGTGGTTCTCTCATAGGTCACAGACCGCGTACCACCTGGGCGGAGAACA
TTAAAAATACAGTCAACATGGTACGCAGGATCATAGGTGATGAAGAAAAATATATGGACTACCTATCG
ACCCAAGTTCGTTACTTGGGTGAAGAAGGTTCTACACCTGGTGTGCTGTAA

SEQ ID NO:7. Vaccine candidate ZIKV-DO-scattered sequence with deoptimized region shown in underline, with locations of nonstructural regions indicated.

(NS1)GATGTAGGGTGCTCGGTAGACTTCTCAAAGAAGGAAACGAGATGCGGTACGGGG
GTATTCGTCTATAACGATGTTGAAGCCTGGCGTGACAGGTACAAATACCATCCTGATTCCCCCCGTCG
ATTGGCAGCAGCGGTCAAGCAAGCGTGGGAAGATGGTATATGCGGGATCTCGTCTGTTTCACGTATGG
AAAACATAATGTGGAGATCGGTAGAAGGGGAGCTAAACGCAATCCTAGAAGAGAATGGTGTTCAACTG
ACGGTAGTTGTAGGATCTGTAAAAAACCCGATGTGGAGAGGTCCGCAGAGATTGCCGGTACCTGTAAA
CGAGCTGCCCCACGGTTGGAAGGCTTGGGGTAAATCGTACTTCGTAAGAGCAGCAAAAACAAATAACT
CGTTTGTCGTGGATGGTGATACACTGAAGGAATGTCCACTCAAACATCGTGCATGGAACTCGTTTCTT
GTAGAGGATCATGGTTTCGGGGTATTTCATACTAGTGTCTGGCTAAAGGTTAGAGAAGATTATTCGTT
AGAGTGTGATCCGGCCGTTATTGGTACAGCTGTTAAAGGAAAGGAGGCGGTACACAGTGATCTAGGTT
ACTGGATTGAAAGTGAGAAGAATGATACATGGAGGCTAAAGAGGGCCCATCTAATCGAGATGAAAACG
TGTGAATGGCCGAAGTCCCACACGTTGTGGACAGATGGTATAGAAGAGTCGGATCTGATCATACCGAA
GTCTTTAGCGGGGCCACTCAGTCATCACAATACGAGAGAGGGCTATAGGACCCAAATGAAAGGTCCAT
GGCACTCGGAAGAGCTTGAAATACGGTTTGAGGAATGTCCAGGCACTAAAGTCCACGTGGAAGAAACA
TGTGGTACAAGAGGACCGTCTCTGAGATCGACCACTGCAAGTGGAAGGGTAATCGAGGAATGGTGTTG
CAGGGAGTGCACGATGCCCCCACTATCGTTCCGGGCGAAAGATGGCTGTTGGTATGGTATGGAGATAC
GTCCCAGGAAAGAACCGGAAAGCAACTTAGTACGTTCAATGGTAACTGCA(NS2A)GGATCGACTGAT
CACATGGATCACTTCTCCCTTGGAGTACTTGTAATCCTGCTCATGGTACAGGAAGGGCTAAAGAAGAG
AATGACGACAAAGATCATAATAAGCACATCGATGGCAGTACTGGTAGCTATGATACTGGGAGGATTTT
CGATGAGTGACCTAGCTAAGCTTGCGATTTTGATGGGTGCGACCTTCGCGGAAATGAATACTGGAGGA
GATGTAGCGCATCTGGCGCTAATAGCGGCATTTAAAGTCAGACCGGCGTTGCTGGTATCGTTCATCTT
CCGTGCTAATTGGACGCCCCGTGAATCGATGCTGCTGGCGTTGGCCTCGTGTCTATTGCAAACTGCGA
TATCCGCCTTGGAAGGTGACCTGATGGTACTCATCAATGGTTTTGCGTTGGCCTGGTTAGCAATACGA
GCGATGGTAGTTCCACGCACGGATAACATCACGTTGGCAATCCTAGCTGCTCTGACGCCACTGGCCCG
TGGCACACTGCTTGTAGCGTGGAGAGCGGGCCTTGCTACGTGCGGGGGGTTTATGCTACTCTCTCTGA
AAGGAAAAGGCAGTGTAAAGAAGAACTTACCGTTTGTCATGGCGCTGGGACTAACGGCTGTAAGGCTG
GTCGATCCCATCAACGTAGTAGGACTGCTGTTACTAACAAGGAGTGGGAAACGG(NS2B)AGCTGGCC
GCCTAGCGAAGTACTAACAGCTGTTGGTCTGATATGCGCATTGGCGGGAGGGTTCGCGAAGGCAGATA
TAGAAATGGCTGGGCCGATGGCCGCGGTAGGTCTGCTAATAGTCAGTTACGTAGTCTCAGGAAAAAGT
GTGGACATGTATATTGAAAGAGCGGGTGACATCACATGGGAAAAAGATGCGGAAGTAACTGGAAACAG
TCCGCGGCTCGATGTAGCGCTAGATGAAAGTGGTGATTTTTCCCTGGTAGAGGATGACGGTCCCCCCA
TGAGAGAAATCATACTCAAAGTAGTCCTGATGACGATCTGTGGCATGAATCCGATAGCCATACCGTTT
GCAGCTGGTGCGTGGTACGTATACGTAAAGACTGGAAAACGT(NS3)AGTGGTGCTCTATGGGATGTA
CCTGCTCCCAAAGAAGTAAAAAAAGGGGAGACCACGGATGGAGTATACAGAGTAATGACGCGTAGACT
GCTAGGTTCGACACAAGTTGGTGTAGGAGTTATGCAAGAAGGGGTCTTTCATACTATGTGGCATGTCA
CAAAAGGTTCCGCGCTGCGTAGCGGTGAAGGTAGACTTGATCCGTACTGGGGAGATGTAAAGCAGGAT
CTAGTATCATACTGTGGTCCGTGGAAGCTAGATGCGGCCTGGGACGGTCACAGCGAGGTACAGCTCTT
GGCGGTACCCCCCGGAGAAAGAGCGAGGAATATCCAGACTCTACCCGGAATATTTAAAACAAAGGATG
GTGACATTGGAGCGGTAGCGCTGGATTATCCAGCAGGAACGTCAGGATCTCCGATCCTAGACAAATGT
GGGAGAGTAATAGGACTTTATGGTAATGGGGTCGTAATCAAAAATGGTAGTTATGTTAGTGCGATCAC
CCAAGGTAGGAGGGAAGAAGAAACTCCTGTTGAATGCTTCGAGCCGTCGATGCTGAAAAAGAAGCAGC
TAACGGTCTTAGACTTACATCCTGGAGCGGGGAAAACCCGAAGAGTTCTTCCGGAAATAGTCCGTGAA
GCGATAAAAACACGTCTCCGTACTGTAATCTTAGCTCCGACCAGGGTTGTAGCTGCTGAAATGGAAGA
GGCCCTTCGTGGGCTTCCAGTACGTTATATGACGACAGCAGTCAATGTAACCCACTCTGGTACAGAAA
TCGTTGACTTAATGTGTCATGCCACCTTTACTTCACGTCTACTACAACCAATCAGAGTTCCCAACTAT
AATCTATATATTATGGATGAAGCCCACTTCACGGATCCCTCAAGTATAGCGGCAAGAGGATATATTTC
AACAAGGGTTGAAATGGGCGAGGCGGCGGCCATCTTCATGACGGCCACGCCACCGGGAACCCGTGATG
CATTTCCGGATTCCAACTCACCGATTATGGACACGGAAGTGGAAGTTCCAGAGAGAGCGTGGAGCTCA
GGTTTTGATTGGGTAACGGATCATTCGGGAAAAACAGTTTGGTTTGTTCCGAGCGTGAGGAATGGCAA
TGAGATAGCAGCTTGTCTAACAAAGGCTGGTAAACGGGTCATACAACTCAGCAGAAAAACTTTTGAAA
CAGAGTTCCAAAAAACAAAACATCAAGAATGGGACTTTGTTGTTACAACTGACATATCAGAGATGGGT
GCCAACTTTAAAGCTGACCGTGTCATAGATTCGAGGAGATGCCTAAAGCCGGTCATACTAGATGGCGA
GCGAGTCATTCTGGCGGGACCCATGCCGGTCACACATGCGAGCGCTGCCCAAAGGAGGGGGCGTATAG
GCAGGAATCCGAACAAACCTGGTGATGAGTATCTATATGGAGGTGGTTGCGCAGAGACGGACGAAGAC
CATGCGCACTGGCTTGAAGCGAGAATGCTCCTAGACAATATTTATCTCCAAGATGGTCTCATAGCCTC
GCTATATCGACCTGAAGCCGACAAAGTAGCGGCCATTGAGGGTGAGTTCAAGCTAAGGACGGAGCAAC
GTAAGACCTTTGTAGAACTCATGAAAAGAGGTGATCTTCCTGTATGGCTGGCCTATCAAGTTGCATCT
GCGGGAATAACCTATACAGATAGAAGATGGTGTTTTGATGGCACGACGAACAACACCATAATGGAAGA
TTCGGTGCCGGCAGAAGTGTGGACCAGACATGGAGAGAAACGTGTGCTCAAACCGAGGTGGATGGATG
CCAGAGTTTGTTCAGATCATGCGGCGCTGAAGTCATTTAAGGAGTTTGCGGCTGGGAAAAGA(NS4A)
GGTGCGGCTTTTGGTGTAATGGAAGCCCTGGGAACACTGCCGGGACACATGACGGAGAGATTCCAAGA
AGCCATTGATAACCTCGCTGTACTCATGCGGGCGGAGACTGGAAGTAGGCCTTACAAAGCCGCGGCGG
CGCAATTGCCGGAAACCCTAGAGACGATAATGCTTTTAGGGTTGCTGGGTACAGTCTCGCTAGGAATC
TTCTTTGTCTTGATGCGTAACAAGGGCATAGGTAAGATGGGCTTTGGTATGGTGACTCTAGGGGCCAG
CGCGTGGCTCATGTGGCTATCGGAAATTGAACCAGCCAGAATAGCATGTGTCCTAATTGTTGTATTCC
TATTGCTGGTAGTACTCATACCTGAACCAGAAAAGCAACGTTCTCCCCAGGATAACCAAATGGCAATA
ATCATCATGGTAGCGGTAGGTCTTCTAGGCTTGATTACGGCC(NS4B)AATGAACTAGGATGGTTGGA
AAGAACAAAGTCGGACCTAAGCCATCTAATGGGTAGGAGAGAGGAAGGGGCAACCATAGGTTTCTCAA
TGGATATTGACCTGCGTCCAGCCTCAGCGTGGGCCATCTATGCGGCCTTGACAACGTTCATTACCCCG
GCCGTCCAACATGCGGTGACCACCTCGTACAACAACTATTCCTTAATGGCGATGGCGACGCAAGCTGG
TGTGTTGTTTGGTATGGGTAAAGGGATGCCGTTCTACGCATGGGATTTTGGAGTCCCGCTACTAATGA
TAGGTTGTTACTCACAATTAACGCCCCTGACCCTAATAGTAGCCATCATTTTACTCGTGGCGCATTAC
ATGTACTTAATCCCAGGGCTACAGGCAGCAGCGGCGCGTGCTGCGCAGAAGAGAACGGCGGCTGGCAT
CATGAAAAACCCTGTTGTAGATGGAATAGTAGTGACTGACATAGACACAATGACGATTGACCCCCAAG
TAGAGAAAAAGATGGGTCAGGTGCTACTAATAGCAGTAGCGGTCTCCAGCGCGATACTGTCGCGGACC
GCCTGGGGTTGGGGGGAGGCGGGGGCCCTGATAACAGCCGCAACGTCCACTTTGTGGGAAGGTTCTCC
GAACAAATACTGGAACTCGTCTACAGCCACGTCACTGTGTAATATTTTTAGGGGTAGTTACTTGGCGG
GAGCTTCTCTAATATACACAGTAACGAGAAACGCTGGTTTGGTCAAGCGTCGT(NS5)GGGGGTGGTA
CAGGAGAGACGCTGGGAGAGAAATGGAAAGCCCGCTTGAATCAGATGTCGGCGCTGGAGTTCTATTCC
TACAAAAAATCAGGCATCACGGAGGTGTGCCGTGAAGAGGCCCGTCGCGCCCTCAAAGACGGTGTGGC
GACGGGAGGCCATGCGGTGTCCCGAGGTAGTGCAAAGCTAAGATGGTTGGTAGAGCGGGGATATCTGC
AGCCCTATGGTAAGGTCATTGATCTAGGATGTGGCCGTGGGGGCTGGTCGTACTACGCCGCGACCATC
CGCAAAGTACAAGAAGTGAAAGGTTACACAAAAGGTGGCCCTGGTCATGAAGAACCCGTGTTGGTGCA
AAGTTATGGGTGGAATATAGTCCGTCTAAAGAGTGGGGTAGACGTCTTTCATATGGCGGCGGAGCCGT
GTGATACGCTGCTGTGTGATATAGGTGAGTCGTCATCTAGTCCGGAAGTGGAAGAAGCGCGGACGCTC
CGTGTCCTCTCCATGGTAGGGGATTGGCTAGAAAAAAGACCGGGAGCCTTTTGTATAAAAGTATTGTG
CCCATATACCAGCACTATGATGGAAACGCTGGAGCGACTACAGCGTAGGTATGGTGGAGGACTGGTAA
GAGTGCCACTATCCCGCAACTCGACACATGAGATGTATTGGGTCTCTGGTGCGAAAAGCAATACCATA
AAAAGTGTATCCACCACGTCGCAGCTCCTCTTAGGGCGCATGGATGGGCCTAGACGTCCAGTGAAATA
TGAAGAGGATGTGAATCTAGGCTCTGGCACGCGTGCTGTGGTAAGTTGCGCTGAAGCGCCCAACATGA
AAATCATTGGTAATCGCATTGAAAGGATACGCAGTGAGCATGCGGAAACGTGGTTTTTTGACGAGAAT
CACCCATATAGGACGTGGGCTTACCATGGTAGCTATGAGGCGCCCACACAAGGTTCAGCGTCCTCGCT
AATAAACGGTGTTGTCAGGCTACTGTCAAAACCGTGGGATGTGGTAACTGGAGTCACGGGAATAGCCA
TGACGGACACCACACCGTATGGTCAACAAAGAGTTTTTAAGGAAAAAGTAGACACTAGGGTACCAGAC
CCCCAAGAAGGTACTCGTCAGGTAATGAGCATGGTATCTTCCTGGTTATGGAAAGAGCTAGGTAAACA
CAAACGTCCACGAGTCTGTACGAAAGAAGAGTTTATCAACAAGGTACGTAGCAATGCGGCATTAGGGG
CGATATTTGAAGAAGAAAAAGAGTGGAAAACTGCAGTGGAAGCGGTGAACGATCCGAGGTTCTGGGCG
CTAGTGGACAAAGAAAGAGAGCATCACCTGAGAGGTGAGTGCCAGTCGTGTGTGTATAATATGATGGG
AAAACGTGAAAAGAAACAAGGTGAATTTGGAAAAGCCAAGGGCAGCCGTGCCATCTGGTATATGTGGC
TAGGTGCTAGATTTCTAGAATTCGAAGCCCTTGGATTCTTGAATGAGGATCACTGGATGGGTAGAGAG
AACTCGGGAGGTGGTGTAGAAGGGCTGGGTTTACAAAGACTAGGATATGTCCTAGAAGAAATGAGTCG
TATACCGGGAGGAAGGATGTATGCGGATGACACTGCGGGCTGGGACACGCGCATTAGCCGTTTTGATC
TGGAAAATGAAGCTCTAATAACCAACCAAATGGAAAAAGGGCACAGGGCGTTGGCATTGGCGATAATC
AAGTATACATACCAAAATAAAGTGGTAAAAGTCCTTAGACCGGCTGAAAAAGGTAAAACAGTTATGGA
TATTATTTCGCGTCAAGACCAAAGGGGTAGCGGACAAGTAGTCACTTACGCGCTTAACACATTTACGA
ACCTAGTGGTACAACTCATTCGTAATATGGAGGCGGAGGAAGTTCTAGAAATGCAAGACTTATGGCTG
CTGCGTAGGTCAGAGAAAGTAACCAACTGGTTACAGAGCAACGGTTGGGATAGGCTAAAACGAATGGC
GGTCAGTGGAGATGATTGTGTTGTGAAGCCGATTGATGATCGTTTTGCACATGCGCTCAGGTTCTTAA
ATGATATGGGTAAAGTTAGAAAAGACACACAAGAATGGAAACCCTCGACTGGATGGGATAACTGGGAA
GAAGTACCGTTTTGCTCGCACCACTTCAATAAGCTCCATCTAAAGGACGGGCGTTCCATTGTGGTACC
CTGCCGCCATCAAGATGAACTAATTGGCCGGGCGCGCGTCTCTCCGGGGGCGGGATGGTCGATCCGGG
AGACGGCTTGCCTAGCGAAATCATATGCGCAAATGTGGCAGCTACTTTATTTCCATAGAAGGGACCTA
CGACTGATGGCGAATGCCATTTGTTCGTCTGTGCCAGTAGACTGGGTTCCGACTGGGAGAACGACCTG
GTCAATACATGGAAAGGGTGAATGGATGACGACTGAAGACATGCTAGTGGTGTGGAATAGAGTGTGGA
TAGAGGAGAACGATCACATGGAAGATAAGACCCCAGTAACGAAATGGACGGACATTCCCTATTTAGGA
AAAAGGGAAGATTTGTGGTGTGGTTCTCTCATAGGTCACAGACCGCGTACCACCTGGGCGGAGAACAT
TAAAAATACAGTCAACATGGTACGCAGGATCATAGGTGATGAAGAAAAATATATGGACTACCTATCGA
CCCAAGTTCGTTACTTGGGTGAAGAAGGTTCTACACCTGGTGTGCTGTAA(NS5 end)

SEQ ID NO:8. Vaccine candidate ZIKV-DO nonstructural region nucleotide sequence, with locations of nonstructural regions indicated. Only regions NS1 to NS3 are shown. In the deoptimized region changed nucleotides are marked in bold and underline.

(NS1)GTCGGTTGTTCGGTAGATTTTTCGAAAAAAGAAACGCGATGTGGTACGGGTGTA
TTTGTATATAATGACGTAGAAGCGTGGCGAGACCGATACAAGTATCATCCGGACTCGCCGCGACGATT
AGCGGCGGCGGTAAAACAAGCGTGGGAAGACGGTATATGCGGTATATCGTCGGTATCGCGAATGGAAA
ATATAATGTGGCGATCGGTAGAAGGTGAGTTAAATGCGATACTAGAAGAGAATGGCGTACAACTAACG
GTAGTAGTCGGCTCGGTAAAAAATCCCATGTGGCGAGGTCCGCAGCGATTGCCCGTCCCCGTCAATGA
GCTACCCCATGGTTGGAAGGCGTGGGGTAAATCGTACTTCGTACGAGCGGCGAAGACGAATAATTCCT
TTGTAGTCGATGGTGACACGCTAAAGGAATGCCCGTTAAAACATCGAGCGTGGAATTCCTTTTTGGTC
GAGGATCATGGTTTCGGTGTATTCCATACCAGTGTATGGTTAAAGGTACGAGAAGATTATTCGTTAGA
GTGTGATCCGGCCGTAATTGGCACGGCGGTAAAGGGCAAGGAGGCGGTACATAGTGATCTCGGTTACT
GGATTGAGAGTGAGAAGAATGACACGTGGCGCCTAAAGCGCGCCCATCTAATAGAGATGAAAACGTGT
GAATGGCCGAAGTCGCACACGTTGTGGACGGATGGCATAGAAGAGAGTGATCTAATAATACCCAAGTC
GTTAGCGGGTCCGTTATCCCATCATAATACCCGAGAGGGTTACCGCACCCAAATGAAAGGTCCGTGGC
ATAGTGAAGAGTTGGAAATTCGGTTCGAGGAATGCCCGGGTACCAAGGTACACGTCGAGGAAACGTGT
GGCACGCGAGGCCCGTCGCTACGATCGACCACCGCGTCCGGCCGCGTCATAGAGGAATGGTGCTGCCG
CGAGTGCACGATGCCCCCGCTATCGTTCCGGGCGAAAGATGGTTGTTGGTATGGAATGGAGATACGCC
CCCGCAAAGAACCGGAATCCAATTTAGTACGCTCGATGGTCACCGCGGGCTCGACCGATCATATGGAC
CATTTCTCGTTG(NS2A)GGCGTCTTGGTCATACTATTAATGGTCCAAGAAGGTCTAAAGAAGCGAAT
GACCACGAAGATAATAATATCCACGTCGATGGCGGTCCTAGTAGCGATGATACTAGGCGGCTTTTCGA
TGAGTGACCTAGCGAAGTTGGCGATTTTGATGGGTGCCACCTTCGCGGAAATGAATACCGGCGGCGAT
GTAGCGCATCTAGCGCTAATAGCGGCGTTCAAAGTACGACCGGCGTTGCTAGTATCGTTCATATTCCG
AGCGAATTGGACGCCCCGTGAATCCATGCTACTAGCCTTGGCCTCGTGTTTGTTGCAAACCGCGATAT
CGGCCTTGGAAGGTGACCTAATGGTATTAATAAATGGTTTCGCGTTGGCCTGGTTGGCGATACGAGCG
ATGGTAGTACCGCGCACCGATAATATAACCTTGGCGATACTAGCGGCGCTAACGCCGCTAGCCCGGGG
TACGCTATTGGTCGCGTGGCGAGCGGGTTTGGCGACCTGCGGTGGTTTTATGTTATTATCGCTAAAGG
GCAAAGGTAGTGTCAAGAAGAATTTACCGTTTGTAATGGCCCTAGGCCTCACCGCGGTCCGCCTAGTA
GACCCCATAAATGTCGTCGGCCTACTATTATTAACGCGCAGTGGTAAGCGGTCCTGGCCC(NS2B)CC
CTCCGAAGTATTAACGGCGGTAGGTCTAATATGCGCGTTGGCGGGCGGTTTCGCCAAGGCGGATATAG
AGATGGCGGGTCCCATGGCCGCGGTAGGTCTACTCATTGTAAGTTACGTCGTATCGGGCAAGAGTGTC
GACATGTACATTGAACGAGCGGGTGACATAACGTGGGAAAAAGATGCGGAAGTAACCGGCAATAGTCC
CCGGTTAGATGTCGCGCTCGATGAGAGTGGTGATTTCTCGCTAGTCGAGGATGACGGTCCCCCGATGC
GAGAGATAATATTAAAGGTCGTACTAATGACCATATGTGGTATGAATCCGATAGCCATACCCTTCGCG
GCGGGCGCGTGGTACGTATACGTCAAGACCGGCAAACGC(NS3)AGTGGTGCGCTCTGGGATGTCCCC
GCGCCCAAGGAAGTAAAAAAGGGTGAGACCACGGATGGCGTCTACCGAGTAATGACCCGTCGACTACT
CGGTTCGACGCAAGTAGGCGTCGGCGTAATGCAAGAGGGTGTATTCCACACCATGTGGCATGTAACGA
AAGGCTCGGCGCTACGATCCGGTGAAGGTCGATTGGATCCGTACTGGGGCGATGTAAAGCAAGATCTA
GTCTCGTACTGTGGTCCGTGGAAGCTCGATGCCGCCTGGGACGGTCACTCCGAGGTCCAGTTATTGGC
CGTCCCCCCGGGCGAGCGAGCGCGCAATATACAAACTCTACCCGGCATATTCAAGACGAAGGATGGTG
ACATTGGCGCGGTAGCGCTAGATTACCCGGCGGGCACTTCGGGCTCGCCGATACTCGACAAGTGTGGT
CGAGTCATAGGCTTGTATGGTAATGGTGTAGTCATAAAAAATGGTAGTTATGTAAGTGCCATAACCCA
AGGTCGCCGCGAAGAAGAGACCCCCGTAGAGTGCTTCGAGCCCTCGATGCTAAAGAAGAAGCAACTCA
CTGTATTAGACTTGCATCCCGGCGCGGGTAAAACCCGCCGAGTATTGCCCGAAATAGTACGTGAAGCC
ATAAAAACGCGATTACGTACCGTCATATTAGCGCCGACCCGCGTAGTAGCGGCGGAAATGGAGGAGGC
CTTGCGAGGTTTGCCGGTCCGTTATATGACGACGGCGGTAAATGTAACCCATTCGGGCACGGAAATAG
TAGACTTAATGTGCCATGCCACCTTCACCTCGCGTCTCCTCCAGCCGATACGAGTACCCAATTATAAT
CTATATATTATGGATGAGGCCCATTTCACGGATCCCTCGAGTATAGCGGCGCGAGGCTACATTTCGAC
GCGCGTAGAGATGGGTGAGGCGGCGGCCATATTCATGACCGCCACGCCGCCGGGCACCCGTGACGCGT
TCCCGGACTCGAATTCGCCGATTATGGACACCGAAGTCGAAGTACCGGAGCGAGCCTGGTCCTCGGGT
TTTGATTGGGTCACGGATCATTCGGGCAAAACGGTATGGTTCGTACCGTCCGTCCGCAATGGTAATGA
GATAGCGGCGTGTCTAACGAAGGCGGGCAAACGGGTAATACAGTTATCCCGAAAGACCTTCGAGACGG
AGTTCCAAAAAACGAAACATCAAGAGTGGGACTTCGTAGTCACGACCGACATTTCGGAGATGGGTGCC
AATTTCAAAGCGGACCGTGTAATAGATTCGCGCCGATGCCTCAAGCCGGTAATATTGGATGGTGAGCG
AGTAATTCTAGCGGGCCCCATGCCCGTAACGCATGCCTCCGCGGCCCAACGCCGCGGTCGCATAGGTC
GCAATCCCAATAAACCCGGCGATGAGTATCTATATGGCGGTGGTTGCGCGGAGACCGACGAAGACCAT
GCGCATTGGTTGGAAGCGCGAATGTTATTGGACAATATTTACTTACAAGATGGTTTAATAGCCTCGTT
ATATCGACCCGAGGCCGACAAAGTAGCGGCCATTGAGGGCGAGTTCAAGTTGCGCACGGAGCAACGCA
AGACCTTCGTCGAATTAATGAAACGAGGCGATTTGCCCGTATGGCTAGCCTATCAAGTAGCGTCGGCC
GGCATAACCTACACGGATCGACGATGGTGCTTCGATGGTACGACCAATAATACCATAATGGAAGATAG
TGTGCCGGC

SEQ ID NO:9. Vaccine candidate ZIKV-DO sequence, with the deoptimized region shown in underline, with locations of nonstructural regions NS1 to NS5 indicated and shown in full.

(NS1)GTCGGTTGTTCGGTAGATTTTTCGAAAAAAGAAACGCGATGTGGTACGGGTGTA
TTTGTATATAATGACGTAGAAGCGTGGCGAGACCGATACAAGTATCATCCGGACTCGCCGCGACGATT
AGCGGCGGCGGTAAAACAAGCGTGGGAAGACGGTATATGCGGTATATCGTCGGTATCGCGAATGGAAA
ATATAATGTGGCGATCGGTAGAAGGTGAGTTAAATGCGATACTAGAAGAGAATGGCGTACAACTAACG
GTAGTAGTCGGCTCGGTAAAAAATCCCATGTGGCGAGGTCCGCAGCGATTGCCCGTCCCCGTCAATGA
GCTACCCCATGGTTGGAAGGCGTGGGGTAAATCGTACTTCGTACGAGCGGCGAAGACGAATAATTCCT
TTGTAGTCGATGGTGACACGCTAAAGGAATGCCCGTTAAAACATCGAGCGTGGAATTCCTTTTTGGTC
GAGGATCATGGTTTCGGTGTATTCCATACCAGTGTATGGTTAAAGGTACGAGAAGATTATTCGTTAGA
GTGTGATCCGGCCGTAATTGGCACGGCGGTAAAGGGCAAGGAGGCGGTACATAGTGATCTCGGTTACT
GGATTGAGAGTGAGAAGAATGACACGTGGCGCCTAAAGCGCGCCCATCTAATAGAGATGAAAACGTGT
GAATGGCCGAAGTCGCACACGTTGTGGACGGATGGCATAGAAGAGAGTGATCTAATAATACCCAAGTC
GTTAGCGGGTCCGTTATCCCATCATAATACCCGAGAGGGTTACCGCACCCAAATGAAAGGTCCGTGGC
ATAGTGAAGAGTTGGAAATTCGGTTCGAGGAATGTCCGGGTACCAAGGTACACGTCGAGGAAACGTGT
GGCACGCGAGGCCCGTCGCTACGATCGACCACCGCGTCCGGCCGCGTCATAGAGGAATGGTGCTGCCG
CGAGTGCACGATGCCCCCGCTATCGTTCCGGGCGAAAGATGGTTGTTGGTATGGAATGGAGATACGCC
CCCGCAAAGAACCGGAATCCAATTTAGTACGCTCGATGGTCACCGCGGGCTCGACCGATCATATGGAC
CATTTCTCGTTG(NS2A)GGCGTCTTGGTCATACTATTAATGGTCCAAGAAGGTCTAAAGAAGCGAAT
GACCACGAAGATAATAATATCCACGTCGATGGCGGTCCTAGTAGCGATGATACTAGGCGGCTTTTCGA
TGAGTGACCTAGCGAAGTTGGCGATTTTGATGGGTGCCACCTTCGCGGAAATGAATACCGGCGGCGAT
GTAGCGCATCTAGCGCTAATAGCGGCGTTCAAAGTACGACCGGCGTTGCTAGTATCGTTCATATTCCG
AGCGAATTGGACGCCCCGTGAATCCATGCTACTAGCCTTGGCCTCGTGTTTGTTGCAAACCGCGATAT
CGGCCTTGGAAGGTGACCTAATGGTATTAATAAATGGTTTCGCGTTGGCCTGGTTGGCGATACGAGCG
ATGGTAGTACCGCGCACCGATAATATAACCTTGGCGATACTAGCGGCGCTAACGCCGCTAGCCCGGGG
TACGCTATTGGTCGCGTGGCGAGCGGGTTTGGCGACCTGCGGTGGTTTTATGTTATTATCGCTAAAGG
GCAAAGGTAGTGTCAAGAAGAATTTACCGTTTGTAATGGCCCTAGGCCTCACCGCGGTCCGCCTAGTA
GACCCCATAAATGTCGTCGGCCTACTATTATTAACGCGCAGTGGTAAGCGGTCCTGGCCC(NS2B)CC
CTCCGAAGTATTAACGGCGGTAGGTCTAATATGCGCGTTGGCGGGCGGTTTCGCCAAGGCGGATATAG
AGATGGCGGGTCCCATGGCCGCGGTAGGTCTACTCATTGTAAGTTACGTCGTATCGGGCAAGAGTGTC
GACATGTACATTGAACGAGCGGGTGACATAACGTGGGAAAAAGATGCGGAAGTAACCGGCAATAGTCC
CCGGTTAGATGTCGCGCTCGATGAGAGTGGTGATTTCTCGCTAGTCGAGGATGACGGTCCCCCGATGC
GAGAGATAATATTAAAGGTCGTACTAATGACCATATGTGGTATGAATCCGATAGCCATACCCTTCGCG
GCGGGCGCGTGGTACGTATACGTCAAGACCGGCAAACGC(NS3)AGTGGTGCGCTCTGGGATGTCCCC
GCGCCCAAGGAAGTAAAAAAGGGTGAGACCACGGATGGCGTCTACCGAGTAATGACCCGTCGACTACT
CGGTTCGACGCAAGTAGGCGTCGGCGTAATGCAAGAGGGTGTATTCCACACCATGTGGCATGTAACGA
AAGGCTCGGCGCTACGATCCGGTGAAGGTCGATTGGATCCGTACTGGGGCGATGTAAAGCAAGATCTA
GTCTCGTACTGTGGTCCGTGGAAGCTCGATGCCGCCTGGGACGGTCACTCCGAGGTCCAGTTATTGGC
CGTCCCGCCGGGCGAGCGAGCGCGCAATATACAAACTCTACCCGGCATATTCAAGACGAAGGATGGTG
ACATTGGCGCGGTAGCGCTAGATTACCCGGCGGGCACTTCGGGCTCGCCGATACTCGACAAGTGTGGT
CGAGTCATAGGCTTGTATGGTAATGGTGTAGTCATAAAAAATGGTAGTTATGTAAGTGCCATAACCCA
AGGTCGCCGCGAAGAAGAGACCCCCGTAGAGTGCTTCGAGCCCTCGATGCTAAAGAAGAAGCAACTCA
CTGTATTAGACTTGCATCCCGGCGCGGGTAAAACCCGCCGAGTATTGCCCGAAATAGTACGTGAAGCC
ATAAAAACGCGATTACGTACCGTCATATTAGCGCCGACCCGCGTAGTAGCGGCGGAAATGGAGGAGGC
CTTGCGAGGTTTGCCGGTCCGTTATATGACGACGGCGGTAAATGTAACCCATTCGGGCACGGAAATAG
TAGACTTAATGTGCCATGCCACCTTCACCTCGCGTCTCCTCCAGCCGATACGAGTACCCAATTATAAT
CTATATATTATGGATGAGGCCCATTTCACGGATCCCTCGAGTATAGCGGCGCGAGGCTACATTTCGAC
GCGCGTAGAGATGGGTGAGGCGGCGGCCATATTCATGACCGCCACGCCGCCGGGCACCCGTGACGCGT
TCCCGGACTCGAATTCGCCGATTATGGACACCGAAGTCGAAGTACCGGAGCGAGCCTGGTCCTCGGGT
TTTGATTGGGTCACGGATCATTCGGGCAAAACGGTATGGTTCGTACCGTCCGTCCGCAATGGTAATGA
GATAGCGGCGTGTCTAACGAAGGCGGGCAAACGGGTAATACAGTTATCCCGAAAGACCTTCGAGACGG
AGTTCCAAAAAACGAAACATCAAGAGTGGGACTTCGTAGTCACGACCGACATTTCGGAGATGGGTGCC
AATTTCAAAGCGGACCGTGTAATAGATTCGCGCCGATGCCTCAAGCCGGTAATATTGGATGGTGAGCG
AGTAATTCTAGCGGGCCCCATGCCCGTAACGCATGCCTCCGCGGCCCAACGCCGCGGTCGCATAGGTC
GCAATCCCAATAAACCCGGCGATGAGTATCTATATGGCGGTGGTTGCGCGGAGACCGACGAAGACCAT
GCGCATTGGTTGGAAGCGCGAATGTTATTGGACAATATTTACTTACAAGATGGTTTAATAGCCTCGTT
ATATCGACCCGAGGCCGACAAAGTAGCGGCCATTGAGGGCGAGTTCAAGTTGCGCACGGAGCAACGCA
AGACCTTCGTCGAATTAATGAAACGAGGCGATTTGCCCGTATGGCTAGCCTATCAAGTAGCGTCGGCA
GGTATAACCTACACGGATCGACGATGGTGCTTCGATGGTACGACCAATAATACCATAATGGAAGATAG
TGTGCCGGCAGAGGTGTGGACCAGACACGGAGAGAAAAGAGTGCTCAAACCGAGGTGGATGGACGCCA
GAGTTTGTTCAGATCATGCGGCCCTGAAGTCATTCAAGGAGTTTGCCGCTGGGAAAAGA(NS4A)GGA
GCGGCTTTTGGAGTGATGGAAGCCCTGGGAACACTGCCAGGACACATGACAGAGAGATTCCAGGAAGC
CATTGACAACCTCGCTGTGCTCATGCGGGCAGAGACTGGAAGCAGGCCTTACAAAGCCGCGGCGGCCC
AATTGCCGGAGACCCTAGAGACCATAATGCTTTTGGGGTTGCTGGGAACAGTCTCGCTGGGAATCTTC
TTCGTCTTGATGAGGAACAAGGGCATAGGGAAGATGGGCTTTGGAATGGTGACTCTTGGGGCCAGCGC
ATGGCTCATGTGGCTCTCGGAAATTGAGCCAGCCAGAATTGCATGTGTCCTCATTGTTGTGTTCCTAT
TGCTGGTGGTGCTCATACCTGAGCCAGAAAAGCAAAGATCTCCCCAGGACAACCAAATGGCAATCATC
ATCATGGTAGCAGTAGGTCTTCTGGGCTTGATTACCGCC(NS4B)AATGAACTCGGATGGTTGGAGAG
AACAAAGAGTGACCTAAGCCATCTAATGGGAAGGAGAGAGGAGGGGGCAACCATAGGATTdTCAATGG
ACATTGACCTGCGGCCAGCCTCAGCTTGGGCCATCTATGCTGCCTTGACAACTTTCATTACCCCAGCC
GTCCAACATGCAGTGACCACCTCATACAACAACTACTCCTTAATGGCGATGGCCACGCAAGCTGGAGT
GTTGTTTGGTATGGGCAAAGGGATGCCATTCTACGCATGGGACTTTGGAGTCCCGCTGCTAATGATAG
GTTGCTACTCACAATTAACACCCCTGACCCTAATAGTGGCCATCATTTTGCTCGTGGCGCACTACATG
TACTTGATCCCAGGGCTGCAGGCAGCAGCTGCGCGTGCTGCCCAGAAGAGAACGGCAGCTGGCATCAT
GAAGAACCCTGTTGTGGATGGAATAGTGGTGACTGACATTGACACAATGACAATTGACCCCCAAGTGG
AGAAAAAGATGGGACAGGTGCTACTCATAGCAGTAGCCGTCTCCAGCGCCATACTGTCGCGGACCGCC
TGGGGGTGGGGGGAGGCTGGGGCCCTGATCACAGCCGCAACTTCCACTTTGTGGGAAGGCTCTCCGAA
CAAGTACTGGAACTCCTCTACAGCCACTTCACTGTGTAACATTTTTAGGGGAAGTTACTTGGCTGGAG
CTTCTCTAATCTACACAGTAACAAGAAACGCTGGCTTGGTCAAGAGACGT(NS5)GGGGGTGGAACAG
GAGAGACCCTGGGAGAGAAATGGAAGGCCCGCTTGAACCAGATGTCGGCCCTGGAGTTCTACTCCTAC
AAAAAGTCAGGCATCACCGAGGTGTGCAGAGAAGAGGCCCGCCGCGCCCTCAAGGACGGTGTGGCAAC
GGGAGGCCATGCTGTGTCCCGAGGAAGTGCAAAGCTGAGATGGTTGGTGGAGCGGGGATACCTGCAGC
CCTATGGAAAGGTCATTGATCTTGGATGTGGCAGAGGGGGCTGGAGTTACTACGCCGCCACCATCCGC
AAAGTTCAAGAAGTGAAAGGATACACAAAAGGAGGCCCTGGTCATGAAGAACCCGTGTTGGTGCAAAG
CTATGGGTGGAACATAGTCCGTCTTAAGAGTGGGGTGGACGTCTTTCATATGGCGGCTGAGCCGTGTG
ACACGCTGCTGTGTGACATAGGTGAGTCATCATCTAGTCCTGAAGTGGAAGAAGCACGGACGCTCAGA
GTCCTCTCCATGGTGGGGGATTGGCTTGAAAAAAGACCAGGAGCCTTTTGTATAAAAGTGTTGTGCCC
ATACACCAGCACTATGATGGAAACCCTGGAGCGACTGCAGCGTAGGTATGGGGGAGGACTGGTCAGAG
TGCCACTCTCCCGCAACTCTACACATGAGATGTACTGGGTCTCTGGAGCGAAAAGCAACACCATAAAA
AGTGTGTCCACCACGAGCCAGCTCCTCTTGGGGCGCATGGACGGGCCTAGAAGGCCAGTGAAATATGA
GGAGGATGTGAATCTCGGCTCTGGCACGCGGGCTGTGGTAAGCTGCGCTGAAGCTCCCAACATGAAGA
TCATTGGTAACCGCATTGAAAGGATCCGCAGTGAGCACGCGGAAACGTGGTTCTTTGACGAGAACCAC
CCATATAGGACATGGGCTTACCATGGAAGCTATGAGGCCCCCACACAAGGGTCAGCGTCCTCTCTAAT
AAACGGGGTTGTCAGGCTCCTGTCAAAACCCTGGGATGTGGTGACTGGAGTCACAGGAATAGCCATGA
CCGACACCACACCGTATGGTCAGCAAAGAGTTTTCAAGGAAAAAGTGGACACTAGGGTGCCAGACCCC
CAAGAAGGCACTCGTCAGGTTATGAGCATGGTCTCTTCCTGGTTGTGGAAAGAGCTAGGCAAACACAA
ACGGCCACGAGTCTGTACCAAAGAAGAGTTCATCAACAAGGTTCGTAGCAATGCAGCATTAGGGGCAA
TATTTGAAGAGGAAAAAGAGTGGAAGACTGCAGTGGAAGCTGTGAACGATCCAAGGTTCTGGGCTCTA
GTGGACAAGGAAAGAGAGCACCACCTGAGAGGAGAGTGCCAGAGTTGTGTGTATAACATGATGGGAAA
AAGAGAAAAGAAACAAGGGGAATTTGGAAAGGCCAAGGGCAGCCGCGCCATCTGGTATATGTGGCTAG
GGGCTAGATTTCTAGAGTTCGAAGCCCTTGGATTCTTGAACGAGGATCACTGGATGGGGAGAGAGAAC
TCAGGAGGTGGTGTTGAAGGGCTGGGATTACAAAGACTCGGATATGTCCTAGAAGAGATGAGTCGTAT
ACCAGGAGGAAGGATGTATGCAGATGACACTGCTGGCTGGGACACCCGCATTAGCAGGTTTGATCTGG
AGAATGAAGCTCTAATCACCAACCAAATGGAGAAAGGGCACAGGGCCTTGGCATTGGCCATAATCAAG
TACACATACCAAAACAAAGTGGTAAAGGTCCTTAGACCAGCTGAAAAAGGGAAAACAGTTATGGACAT
TATTTCGAGACAAGACCAAAGGGGGAGCGGACAAGTTGTCACTTACGCTCTTAACACATTTACCAACC
TAGTGGTGCAACTCATTCGGAATATGGAGGCTGAGGAAGTTCTAGAGATGCAAGACTTGTGGCTGCTG
CGGAGGTCAGAGAAAGTGACCAACTGGTTGCAGAGCAACGGATGGGATAGGCTCAAACGAATGGCAGT
CAGTGGAGATGATTGCGTTGTGAAGCCAATTGATGATAGGTTTGCACATGCCCTCAGGTTCTTGAATG
ATATGGGAAAAGTTAGAAAGGACACACAAGAGTGGAAACCCTCAACTGGATGGGACAACTGGGAAGAA
GTTCCGTTTTGCTCCCACCACTTCAACAAGCTCCATCTCAAGGACGGGAGGTCCATTGTGGTTCCCTG
CCGCCACCAAGATGAACTGATTGGCCGGGCCCGCGTCTCTCCAGGGGCGGGATGGAGCATCCGGGAGA
CTGCTTGCCTAGCAAAATCATATGCGCAGATGTGGCAGCTCCTTTATTTCCACAGAAGGGACCTCCGA
CTGATGGCCAATGCCATTTGTTCATCTGTGCCAGTTGACTGGGTTCCAACTGGGAGAACTACCTGGTC
AATCCATGGAAAGGGAGAATGGATGACCACTGAAGACATGCTTGTGGTGTGGAACAGAGTGTGGATTG
AGGAGAACGACCACATGGAAGACAAGACCCCAGTTACGAAATGGACAGACATTCCCTATTTGGGAAAA
AGGGAAGACTTGTGGTGTGGATCTCTCATAGGGCACAGACCGCGCACCACCTGGGCTGAGAACATTAA
AAACACAGTCAACATGGTGCGCAGGATCATAGGTGATGAAGAAAAGTACATGGACTACCTATCCACCC
AAGTTCGCTACTTGGGTGAAGAAGGGTCTACACCTGGAGTGCTGTAA(NS5 end)

SEQ ID NO:10. Vaccine candidate ZIKV-DO-NS3, more extensive sequence of flanking regions, with the deoptimized region shown in underline, with positions of key regions indicated.

AGTTGTTGATCTGTGTGAATCAGACTGCGACAGTTCGAGTTTGAAGCGAAAGCTAGCAA
CAGTATCAACAGGTTTTATTTTGGATTTGGAAACGAGAGTTTCTGGTCATGAAAAACCCAAAAAAGAA
ATCCGGAGGATTCCGGATTGTCAATATGCTAAAACGCGGAGTAGCCCGTGTGAGCCCCTTTGGGGGCT
TGAAGAGGCTGCCAGCCGGACTTCTGCTGGGTCATGGGCCCATCAGGATGGTCTTGGCGATTCTAGCC
TTTTTGAGATTCACGGCAATCAAGCCATCACTGGGTCTCATCAATAGATGGGGTTCAGTTGGGAAAAA
AGAGGCTATGGAAATAATAAAGAAGTTCAAGAAAGATCTGGCTGCCATGCTGAGAATAATCAATGCTA
GGAAGGAGAAGAAGAGACGAGGCGCAGATACTAGTGTCGGAATTGTTGGCCTCCTGCTGACCACAGCT
ATGGCAGCGGAGGTCACTAGACGTGGGAGTGCATACTATATGTACTTGGACAGAAACGATGCTGGGGA
GGCCATATCTTTTCCAACCACATTGGGGATGAATAAGTGTTATATACAGATCATGGATCTTGGACACA
TGTGTGATGCCACCATGAGCTATGAATGCCCTATGCTGGATGAGGGGGTGGAACCAGATGACGTCGAT
TGTTGGTGCAACACGACGTCAACTTGGGTTGTGTACGGAACCTGCCATCACAAAAAAGGTGAAGCACG
GAGATCTAGAAGAGCTGTGACGCTCCCCTCCCATTCCACTAGGAAGCTGCAAACGCGGTCGCAAACCT
GGTTGGAATCAAGAGAATACACAAAGCACTTGATTAGAGTCGAAAATTGGATATTCAGGAACCCTGGC
TTCGCGTTAGCAGCAGCTGCCATCGCTTGGCTTTTGGGAAGCTCAACGAGCCAAAAAGTCATATACTT
GGTCATGATACTGCTGATTGCCCCGGCATACAGCATCAGGTGCATAGGAGTCAGCAATAGGGACTTTG
TGGAAGGTATGTCAGGTGGGACTTGGGTTGATGTTGTCTTGGAACATGGAGGTTGTGTCACCGTAATG
GCACAGGACAAACCGACTGTCGACATAGAGCTGGTTACAACAACAGTCAGCAACATGGCGGAGGTAAG
ATCCTACTGCTATGAGGCATCAATATCAGACATGGCTTCGGACAGCCGCTGCCCAACACAAGGTGAAG
CCTACCTTGACAAGCAATCAGACACTCAATATGTCTGCAAAAGAACGTTAGTGGACAGAGGCTGGGGA
AATGGATGTGGACTTTTTGGCAAAGGGAGCCTGGTGACATGCGCTAAGTTTGCATGCTCCAAGAAAAT
GACCGGGAAGAGCATCCAGCCAGAGAATCTGGAGTACCGGATAATGCTGTCAGTTCATGGCTCCCAGC
ACAGTGGGATGATCGTTAATGACACAGGACATGAAACTGATGAGAATAGAGCGAAAGTTGAGATAACG
CCCAATTCACCAAGAGCCGAAGCCACCCTGGGGGGTTTTGGAAGCCTAGGACTTGATTGTGAACCGAG
GACAGGCCTTGACTTTTCAGATTTGTATTACTTGACTATGAATAACAAGCACTGGTTGGTTCACAAGG
AGTGGTTCCACGACATTCCATTACCTTGGCACGCTGGGGCAGACACCGGAACTCCACACTGGAACAAC
AAAGAAGCACTGGTAGAGTTCAAGGACGCACATGCCAAAAGGCAAACTGTCGTGGTTCTAGGGAGTCA
AGAAGGAGCAGTTCACACGGCCCTTGCTGGAGCTCTGGAGGCTGAGATGGATGGTGCAAAGGGAAGGC
TGTCCTCTGGCCACTTGAAATGTCGCCTGAAAATGGATAAACTTAGATTGAAGGGCGTGTCATACTCC
TTGTGTACTGCAGCGTTCACATTCACCAAGATCCCGGCTGAAACACTGCACGGGACAGTCACAGTGGA
GGTACAGTACGCAGGGACAGATGGACCTTGCAAGGTTCCAGCTCAGATGGCGGTGGACATGCAAACTC
TGACCCCAGTTGGGAGGTTGATAACCGCTAACCCCGTAATCACTGAAAGCACTGAGAACTCTAAGATG
ATGCTGGAACTTGATCCACCATTTGGGGACTCTTACATTGTCATAGGAGTCGGGGAGAAGAAGATCAC
CCACCACTGGCACAGGAGTGGCAGCACCATTGGAAAAGCATTTGAAGCCACTGTGAGAGGTGCCAAGA
GAATGGCAGTCTTGGGAGACACAGCCTGGGACTTTGGATCAGTTGGAGGCGCTCTCAACTCATTGGGC
AAGGGCATCCATCAAATTTTTGGAGCAGCTTTCAAATCATTGTTTGGAGGAATGTCCTGGTTCTCACA
AATTCTCATTGGAACGTTGCTGATGTGGTTGGGTCTGAACACAAAGAATGGATCTATTTCCCTTATGT
GCTTGGCCTTAGGGGGGGTGTTGATCTTCTTATCCACAGCCGTCTCTGCT(NS1)GATGTGGGGTGCT
CGGTGGACTTCTCAAAGAAGGAGACGAGATGCGGTACAGGGGTGTTCGTCTATAACGACGTTGAAGCC
TGGAGGGACAGGTACAAGTACCATCCTGACTCCCCCCGTAGATTGGCAGCAGCAGTCAAGCAAGCCTG
GGAAGATGGTATCTGCGGGATCTCCTCTGTTTCAAGAATGGAAAACATCATGTGGAGATCAGTAGAAG
GGGAGCTCAACGCAATCCTGGAAGAGAATGGAGTTCAACTGACGGTCGTTGTGGGATCTGTAAAAAAC
CCCATGTGGAGAGGTCCACAGAGATTGCCCGTGCCTGTGAACGAGCTGCCCCACGGCTGGAAGGCTTG
GGGGAAATCGTACTTCGTCAGAGCAGCAAAGACAAATAACAGCTTTGTCGTGGATGGTGACACACTGA
AGGAATGCCCACTCAAACATAGAGCATGGAACAGCTTTCTTGTGGAGGATCATGGGTTCGGGGTATTT
CACACTAGTGTCTGGCTCAAGGTTAGAGAAGATTATTCATTAGAGTGTGATCCAGCCGTTATTGGAAC
AGCTGTTAAGGGAAAGGAGGCTGTACACAGTGATCTAGGCTACTGGATTGAGAGTGAGAAGAATGACA
CATGGAGGCTGAAGAGGGCCCATCTGATCGAGATGAAAACATGTGAATGGCCAAAGTCCCACACATTG
TGGACAGATGGAATAGAAGAGAGTGATCTGATCATACCCAAGTCTTTAGCTGGGCCACTCAGCCATCA
CAATACCAGAGAGGGCTACAGGACCCAAATGAAAGGGCCATGGCACAGTGAAGAGCTTGAAATTCGGT
TTGAGGAATGCCCAGGCACTAAGGTCCACGTGGAGGAAACATGTGGAACAAGAGGACCATCTCTGAGA
TCAACCACTGCAAGCGGAAGGGTGATCGAGGAATGGTGCTGCAGGGAGTGCACAATGCCCCCACTGTC
GTTCCGGGCTAAAGATGGCTGTTGGTATGGAATGGAGATAAGGCCCAGGAAAGAACCAGAAAGCAACT
TAGTAAGGTCAATGGTGACTGCA(NS2A)GGATCAACTGATCACATGGACCACTTCTCCCTTGGAGTG
CTTGTGATCCTGCTCATGGTGCAGGAAGGGCTGAAGAAGAGAATGACCACAAAGATCATCATAAGCAC
ATCAATGGCAGTGCTGGTAGCTATGATCCTGGGAGGATTTTCAATGAGTGACCTGGCTAAGCTTGCAA
TTTTGATGGGTGCCACCTTCGCGGAAATGAACACTGGAGGAGATGTAGCTCATCTGGCGCTGATAGCG
GCATTCAAAGTCAGACCAGCGTTGCTGGTATCTTTCATCTTCAGAGCTAATTGGACACCCCGTGAAAG
CATGCTGCTGGCCTTGGCCTCGTGTCTTTTGCAAACTGCGATCTCCGCCTTGGAAGGCGACCTGATGG
TTCTCATCAATGGTTTTGCTTTGGCCTGGTTGGCAATACGAGCGATGGTTGTTCCACGCACTGATAAC
ATCACCTTGGCAATCCTGGCTGCTCTGACACCACTGGCCCGGGGCACACTGCTTGTGGCGTGGAGAGC
AGGCCTTGCTACTTGCGGGGGGTTTATGCTCCTCTCTCTGAAGGGAAAAGGCAGTGTGAAGAAGAACT
TACCATTTGTCATGGCCCTGGGACTAACCGCTGTGAGGCTGGTCGACCCCATCAACGTGGTGGGACTG
CTGTTACTCACAAGGAGTGGGAAGCGG(NS2B)AGCTGGCCCCCTAGCGAAGTACTCACAGCTGTTGG
CCTGATATGCGCATTGGCTGGAGGGTTCGCCAAGGCAGATATAGAGATGGCTGGGCCCATGGCCGCGG
TCGGTCTGCTAATTGTCAGTTACGTGGTCTCAGGAAAGAGTGTGGACATGTACATTGAAAGAGCAGGT
GACATCACATGGGAAAAAGATGCGGAAGTCACTGGAAACAGTCCCCGGCTCGATGTGGCGCTAGATGA
GAGTGGTGATTTCTCCCTGGTGGAGGATGACGGTCCCCCCATGAGAGAGATCATACTCAAGGTGGTCC
TGATGACCATCTGTGGCATGAATCCAATAGCCATACCCTTTGCAGCTGGAGCGTGGTACGTATACGTG
AAGACTGGAAAAAGG(NS3)AGTGGTGCGCTCTGGGATGTCCCCGCGCCCAAGGAAGTAAAAAAGGGT
GAGACCACGGATGGCGTCTACCGAGTAATGACCCGTCGACTACTCGGTTCGACGCAAGTAGGCGTCGG
CGTAATGCAAGAGGGTGTATTCCACACCATGTGGCATGTAACGAAAGGCTCGGCGCTACGATCCGGTG
AAGGTCGATTGGATCCGTACTGGGGCGATGTAAAGCAAGATCTAGTCTCGTACTGTGGTCCGTGGAAG
CTCGATGCCGCCTGGGACGGTCACTCCGAGGTCCAGTTATTGGCCGTCCCGCCGGGCGAGCGAGCGCG
CAATATACAAACTCTACCCGGCATATTCAAGACGAAGGATGGTGACATTGGCGCGGTAGCGCTAGATT
ACCCGGCGGGCACTTCGGGCTCGCCGATACTCGACAAGTGTGGTCGAGTCATAGGCTTGTATGGTAAT
GGTGTAGTCATAAAAAATGGTAGTTATGTAAGTGCCATAACCCAAGGTCGCCGCGAAGAAGAGACCCC
CGTAGAGTGCTTCGAGCCCTCGATGCTAAAGAAGAAGCAACTCACTGTATTAGACTTGCATCCCGGCG
CGGGTAAAACCCGCCGAGTATTGCCCGAAATAGTACGTGAAGCCATAAAAACGCGATTACGTACCGTC
ATATTAGCGCCGACCCGCGTAGTAGCGGCGGAAATGGAGGAGGCCTTGCGAGGTTTGCCAGTCCGTTA
TATGACGACGGCGGTAAATGTAACCCATTCGGGCACGGAAATAGTAGACTTAATGTGCCATGCCACCT
TCACCTCGCGTCTCCTCCAGCCGATACGAGTACCCAATTATAATCTATATATTATGGATGAGGCCCAT
TTCACGGATCCCTCGAGTATAGCGGCGCGAGGCTACATTTCGACGCGCGTAGAGATGGGTGAGGCGGC
GGCCATATTCATGACCGCCACGCCGCCGGGCACCCGTGACGCGTTCCCGGACTCGAATTCGCCGATTA
TGGACACCGAAGTCGAAGTACCGGAGCGAGCCTGGTCCTCGGGTTTTGATTGGGTCACGGATCATTCG
GGCAAAACGGTATGGTTCGTACCGTCCGTCCGCAATGGTAATGAGATAGCGGCGTGTCTAACGAAGGC
GGGCAAACGGGTAATACAGTTATCCCGAAAGACCTTCGAGACGGAGTTCCAAAAAACGAAACATCAAG
AGTGGGACTTCGTAGTCACGACCGACATTTCGGAGATGGGTGCCAATTTCAAAGCAGACCGTGTAATA
GATTCGCGCCGATGCCTCAAGCCGGTAATATTGGATGGTGAGCGAGTAATTCTAGCGGGCCCCATGCC
CGTAACGCATGCCTCCGCGGCCCAACGCCGCGGTCGCATAGGTCGCAATCCCAATAAACCCGGCGATG
AGTATCTATATGGCGGTGGTTGCGCGGAGACCGACGAAGACCATGCGCATTGGTTGGAAGCGCGAATG
TTATTGGACAATATTTACTTACAAGATGGTTTAATAGCCTCGTTATATCGACCCGAGGCCGACAAAGT
AGCGGCCATTGAGGGCGAGTTCAAGTTGCGCACGGAGCAACGCAAGACCTTCGTCGAATTAATGAAAC
GAGGCGATTTGCCCGTATGGCTAGCCTATCAAGTAGCGTCGGCAGGTATAACCTACACGGATCGACGA
TGGTGCTTCGATGGTACGACCAATAATACCATAATGGAAGATAGTGTGCCGGCAGAGGTGTGGACCAG
ACACGGAGAGAAAAGAGTGCTCAAACCGAGGTGGATGGACGCCAGAGTTTGTTCAGATCATGCGGCCC
TGAAGTCATTCAAGGAGTTTGCCGCTGGGAAAAGA(NS4A)GGAGCGGCTTTTGGAGTGATGGAAGCC
CTGGGAACACTGCCAGGACACATGACAGAGAGATTCCAGGAAGCCATTGACAACCTCGCTGTGCTCAT
GCGGGCAGAGACTGGAAGCAGGCCTTACAAAGCCGCGGCGGCCCAATTGCCGGAGACCCTAGAGACCA
TAATGCTTTTGGGGTTGCTGGGAACAGTCTCGCTGGGAATCTTCTTCGTCTTGATGAGGAACAAGGGC
ATAGGGAAGATGGGCTTTGGAATGGTGACTCTTGGGGCCAGCGCATGGCTCATGTGGCTCTCGGAAAT
TGAGCCAGCCAGAATTGCATGTGTCCTCATTGTTGTGTTCCTATTGCTGGTGGTGCTCATACCTGAGC
CAGAAAAGCAAAGATCTCCCCAGGACAACCAAATGGCAATCATCATCATGGTAGCAGTAGGTCTTCTG
GGCTTGATTACCGCC(NS4B)AATGAACTCGGATGGTTGGAGAGAACAAAGAGTGACCTAAGCCATCT
AATGGGAAGGAGAGAGGAGGGGGCAACCATAGGATTCTCAATGGACATTGACCTGCGGCCAGCCTCAG
CTTGGGCCATCTATGCTGCCTTGACAACTTTCATTACCCCAGCCGTCCAACATGCAGTGACCACCTCA
TACAACAACTACTCCTTAATGGCGATGGCCACGCAAGCTGGAGTGTTGTTTGGTATGGGCAAAGGGAT
GCCATTCTACGCATGGGACTTTGGAGTCCCGCTGCTAATGATAGGTTGCTACTCACAATTAACACCCC
TGACCCTAATAGTGGCCATCATTTTGCTCGTGGCGCACTACATGTACTTGATCCCAGGGCTGCAGGCA
GCAGCTGCGCGTGCTGCCCAGAAGAGAACGGCAGCTGGCATCATGAAGAACCCTGTTGTGGATGGAAT
AGTGGTGACTGACATTGACACAATGACAATTGACCCCCAAGTGGAGAAAAAGATGGGACAGGTGCTAC
TCATAGCAGTAGCCGTCTCCAGCGCCATACTGTCGCGGACCGCCTGGGGGTGGGGGGAGGCTGGGGCC
CTGATCACAGCCGCAACTTCCACTTTGTGGGAAGGCTCTCCGAACAAGTACTGGAACTCCTCTACAGC
CACTTCACTGTGTAACATTTTTAGGGGAAGTTACTTGGCTGGAGCTTCTCTAATCTACACAGTAACAA
GAAACGCTGGCTTGGTCAAGAGACGT(NS5)GGGGGTGGAACAGGAGAGACCCTGGGAGAGAAATGGA
AGGCCCGCTTGAACCAGATGTCGGCCCTGGAGTTCTACTCCTACAAAAAGTCAGGCATCACCGAGGTG
TGCAGAGAAGAGGCCCGCCGCGCCCTCAAGGACGGTGTGGCAACGGGAGGCCATGCTGTGTCCCGAGG
AAGTGCAAAGCTGAGATGGTTGGTGGAGCGGGGATACCTGCAGCCCTATGGAAAGGTCATTGATCTTG
GATGTGGCAGAGGGGGCTGGAGTTACTACGCCGCCACCATCCGCAAAGTTCAAGAAGTGAAAGGATAC
ACAAAAGGAGGCCCTGGTCATGAAGAACCCGTGTTGGTGCAAAGCTATGGGTGGAACATAGTCCGTCT
TAAGAGTGGGGTGGACGTCTTTCATATGGCGGCTGAGCCGTGTGACACGCTGCTGTGTGACATAGGTG
AGTCATCATCTAGTCCTGAAGTGGAAGAAGCACGGACGCTCAGAGTCCTCTCCATGGTGGGGGATTGG
CTTGAAAAAAGACCAGGAGCCTTTTGTATAAAAGTGTTGTGCCCATACACCAGCACTATGATGGAAAC
CCTGGAGCGACTGCAGCGTAGGTATGGGGGAGGACTGGTCAGAGTGCCACTCTCCCGCAACTCTACAC
ATGAGATGTACTGGGTCTCTGGAGCGAAAAGCAACACCATAAAAAGTGTGTCCACCACGAGCCAGCTC
CTCTTGGGGCGCATGGACGGGCCTAGAAGGCCAGTGAAATATGAGGAGGATGTGAATCTCGGCTCTGG
CACGCGGGCTGTGGTAAGCTGCGCTGAAGCTCCCAACATGAAGATCATTGGTAACCGCATTGAAAGGA
TCCGCAGTGAGCACGCGGAAACGTGGTTCTTTGACGAGAACCACCCATATAGGACATGGGCTTACCAT
GGAAGCTATGAGGCCCCCACACAAGGGTCAGCGTCCTCTCTAATAAACGGGGTTGTCAGGCTCCTGTC
AAAACCCTGGGATGTGGTGACTGGAGTCACAGGAATAGCCATGACCGACACCACACCGTATGGTCAGC
AAAGAGTTTTCAAGGAAAAAGTGGACACTAGGGTGCCAGACCCCCAAGAAGGCACTCGTCAGGTTATG
AGCATGGTCTCTTCCTGGTTGTGGAAAGAGCTAGGCAAACACAAACGGCCACGAGTCTGTACCAAAGA
AGAGTTCATCAACAAGGTTCGTAGCAATGCAGCATTAGGGGCAATATTTGAAGAGGAAAAAGAGTGGA
AGACTGCAGTGGAAGCTGTGAACGATCCAAGGTTCTGGGCTCTAGTGGACAAGGAAAGAGAGCACCAC
CTGAGAGGAGAGTGCCAGAGTTGTGTGTATAACATGATGGGAAAAAGAGAAAAGAAACAAGGGGAATT
TGGAAAGGCCAAGGGCAGCCGCGCCATCTGGTATATGTGGCTAGGGGCTAGATTTCTAGAGTTCGAAG
CCCTTGGATTCTTGAACGAGGATCACTGGATGGGGAGAGAGAACTCAGGAGGTGGTGTTGAAGGGCTG
GGATTACAAAGACTCGGATATGTCCTAGAAGAGATGAGTCGTATACCAGGAGGAAGGATGTATGCAGA
TGACACTGCTGGCTGGGACACCCGCATTAGCAGGTTTGATCTGGAGAATGAAGCTCTAATCACCAACC
AAATGGAGAAAGGGCACAGGGCCTTGGCATTGGCCATAATCAAGTACACATACCAAAACAAAGTGGTA
AAGGTCCTTAGACCAGCTGAAAAAGGGAAAACAGTTATGGACATTATTTCGAGACAAGACCAAAGGGG
GAGCGGACAAGTTGTCACTTACGCTCTTAACACATTTACCAACCTAGTGGTGCAACTCATTCGGAATA
TGGAGGCTGAGGAAGTTCTAGAGATGCAAGACTTGTGGCTGCTGCGGAGGTCAGAGAAAGTGACCAAC
TGGTTGCAGAGCAACGGATGGGATAGGCTCAAACGAATGGCAGTCAGTGGAGATGATTGCGTTGTGAA
GCCAATTGATGATAGGTTTGCACATGCCCTCAGGTTCTTGAATGATATGGGAAAAGTTAGAAAGGACA
CACAAGAGTGGAAACCCTCAACTGGATGGGACAACTGGGAAGAAGTTCCGTTTTGCTCCCACCACTTC
AACAAGCTCCATCTCAAGGACGGGAGGTCCATTGTGGTTCCCTGCCGCCACCAAGATGAACTGATTGG
CCGGGCCCGCGTCTCTCCAGGGGCGGGATGGAGCATCCGGGAGACTGCTTGCCTAGCAAAATCATATG
CGCAGATGTGGCAGCTCCTTTATTTCCACAGAAGGGACCTCCGACTGATGGCCAATGCCATTTGTTCA
TCTGTGCCAGTTGACTGGGTTCCAACTGGGAGAACTACCTGGTCAATCCATGGAAAGGGAGAATGGAT
GACCACTGAAGACATGCTTGTGGTGTGGAACAGAGTGTGGATTGAGGAGAACGACCACATGGAAGACA
AGACCCCAGTTACGAAATGGACAGACATTCCCTATTTGGGAAAAAGGGAAGACTTGTGGTGTGGATCT
CTCATAGGGCACAGACCGCGCACCACCTGGGCTGAGAACATTAAAAACACAGTCAACATGGTGCGCAG
GATCATAGGTGATGAAGAAAAGTACATGGACTACCTATCCACCCAAGTTCGCTACTTGGGTGAAGAAG
GGTCTACACCTGGAGTGCTGTAA(NS5 end)
GCACCAATCTTAATGTTGTCAGGCCTGCTAGTCAGCCACAGCTTGGGGAAAGCTGTGCAGCCTGTGAC
CCCCCAGGAGAAGCTGGGAAACCAAGCCTATAGTCAGGCCGAGAACGCCATGGCACGGAAGAAGCCAT
GCTGCCTGTGAGCCCCTCAGAGGATACTGAGTCAAAAAACCCCACGCGCTTGGAGGCGCAGGATGGGA
AAAGAAGGTGGCGACCTTCCCCACCCTTCAATCTGGGGCCTGAACTGGAGATCAGCTGTGGATCCCCA
GAAGAGGGACTAGTGGTTAGAGGAGACCCCCCGGAAAACGCAAAACAGCATATTGACGCTGGGAAAGA
CCAGAGACTCCATGAGTTTCCACCACGCTGGCCGCCAGGCACAGATCGCCGAACTTCGGCGGCCGGTG
TGGGGAAATCCATGGTTTCT

SEQ ID NO:11. Vaccine candidate ZIKV-DO-scattered, more extensive sequence of flanking regions, with deoptimized region shown in underline, with locations of key regions indicated.

AGTTGTTGATCTGTGTGAATCAGACTGCGACAGTTCGAGTTTGAAGCGAAAGCTAGCAA
CAGTATCAACAGGTTTTATTTTGGATTTGGAAACGAGAGTTTCTGGTCATGAAAAACCCAAAAAAGAA
ATCCGGAGGATTCCGGATTGTCAATATGCTAAAACGCGGAGTAGCCCGTGTGAGCCCCTTTGGGGGCT
TGAAGAGGCTGCCAGCCGGACTTCTGCTGGGTCATGGGCCCATCAGGATGGTCTTGGCGATTCTAGCC
TTTTTGAGATTCACGGCAATCAAGCCATCACTGGGTCTCATCAATAGATGGGGTTCAGTTGGGAAAAA
AGAGGCTATGGAAATAATAAAGAAGTTCAAGAAAGATCTGGCTGCCATGCTGAGAATAATCAATGCTA
GGAAGGAGAAGAAGAGACGAGGCGCAGATACTAGTGTCGGAATTGTTGGCCTCCTGCTGACCACAGCT
ATGGCAGCGGAGGTCACTAGACGTGGGAGTGCATACTATATGTACTTGGACAGAAACGATGCTGGGGA
GGCCATATCTTTTCCAACCACATTGGGGATGAATAAGTGTTATATACAGATCATGGATCTTGGACACA
TGTGTGATGCCACCATGAGCTATGAATGCCCTATGCTGGATGAGGGGGTGGAACCAGATGACGTCGAT
TGTTGGTGCAACACGACGTCAACTTGGGTTGTGTACGGAACCTGCCATCACAAAAAAGGTGAAGCACG
GAGATCTAGAAGAGCTGTGACGCTCCCCTCCCATTCCACTAGGAAGCTGCAAACGCGGTCGCAAACCT
GGTTGGAATCAAGAGAATACACAAAGCACTTGATTAGAGTCGAAAATTGGATATTCAGGAACCCTGGC
TTCGCGTTAGCAGCAGCTGCCATCGCTTGGCTTTTGGGAAGCTCAACGAGCCAAAAAGTCATATACTT
GGTCATGATACTGCTGATTGCCCCGGCATACAGCATCAGGTGCATAGGAGTCAGCAATAGGGACTTTG
TGGAAGGTATGTCAGGTGGGACTTGGGTTGATGTTGTCTTGGAACATGGAGGTTGTGTCACCGTAATG
GCACAGGACAAACCGACTGTCGACATAGAGCTGGTTACAACAACAGTCAGCAACATGGCGGAGGTAAG
ATCCTACTGCTATGAGGCATCAATATCAGACATGGCTTCGGACAGCCGCTGCCCAACACAAGGTGAAG
CCTACCTTGACAAGCAATCAGACACTCAATATGTCTGCAAAAGAACGTTAGTGGACAGAGGCTGGGGA
AATGGATGTGGACTTTTTGGCAAAGGGAGCCTGGTGACATGCGCTAAGTTTGCATGCTCCAAGAAAAT
GACCGGGAAGAGCATCCAGCCAGAGAATCTGGAGTACCGGATAATGCTGTCAGTTCATGGCTCCCAGC
ACAGTGGGATGATCGTTAATGACACAGGACATGAAACTGATGAGAATAGAGCGAAAGTTGAGATAACG
CCCAATTCACCAAGAGCCGAAGCCACCCTGGGGGGTTTTGGAAGCCTAGGACTTGATTGTGAACCGAG
GACAGGCCTTGACTTTTCAGATTTGTATTACTTGACTATGAATAACAAGCACTGGTTGGTTCACAAGG
AGTGGTTCCACGACATTCCATTACCTTGGCACGCTGGGGCAGACACCGGAACTCCACACTGGAACAAC
AAAGAAGCACTGGTAGAGTTCAAGGACGCACATGCCAAAAGGCAAACTGTCGTGGTTCTAGGGAGTCA
AGAAGGAGCAGTTCACACGGCCCTTGCTGGAGCTCTGGAGGCTGAGATGGATGGTGCAAAGGGAAGGC
TGTCCTCTGGCCACTTGAAATGTCGCCTGAAAATGGATAAACTTAGATTGAAGGGCGTGTCATACTCC
TTGTGTACTGCAGCGTTCACATTCACCAAGATCCCGGCTGAAACACTGCACGGGACAGTCACAGTGGA
GGTACAGTACGCAGGGACAGATGGACCTTGCAAGGTTCCAGCTCAGATGGCGGTGGACATGCAAACTC
TGACCCCAGTTGGGAGGTTGATAACCGCTAACCCCGTAATCACTGAAAGCACTGAGAACTCTAAGATG
ATGCTGGAACTTGATCCACCATTTGGGGACTCTTACATTGTCATAGGAGTCGGGGAGAAGAAGATCAC
CCACCACTGGCACAGGAGTGGCAGCACCATTGGAAAAGCATTTGAAGCCACTGTGAGAGGTGCCAAGA
GAATGGCAGTCTTGGGAGACACAGCCTGGGACTTTGGATCAGTTGGAGGCGCTCTCAACTCATTGGGC
AAGGGCATCCATCAAATTTTTGGAGCAGCTTTCAAATCATTGTTTGGAGGAATGTCCTGGTTCTCACA
AATTCTCATTGGAACGTTGCTGATGTGGTTGGGTCTGAACACAAAGAATGGATCTATTTCCCTTATGT
GCTTGGCCTTAGGGGGGGTGTTGATCTTCTTATCCACAGCCGTCTCTGCT(NS1)GATGTAGGGTGCT
CGGTAGACTTCTCAAAGAAGGAAACGAGATGCGGTACGGGGGTATTCGTCTATAACGATGTTGAAGCC
TGGCGTGACAGGTACAAATACCATCCTGATTCCCCCCGTCGATTGGCAGCAGCGGTCAAGCAAGCGTG
GGAAGATGGTATATGCGGGATCTCGTCTGTTTCACGTATGGAAAACATAATGTGGAGATCGGTAGAAG
GGGAGCTAAACGCAATCCTAGAAGAGAATGGTGTTCAACTGACGGTAGTTGTAGGATCTGTAAAAAAC
CCGATGTGGAGAGGTCCGCAGAGATTGCCGGTACCTGTAAACGAGCTGCCCCACGGTTGGAAGGCTTG
GGGTAAATCGTACTTCGTAAGAGCAGCAAAAACAAATAACTCGTTTGTCGTGGATGGTGATACACTGA
AGGAATGTCCACTCAAACATCGTGCATGGAACTCGTTTCTTGTAGAGGATCATGGTTTCGGGGTATTT
CATACTAGTGTCTGGCTAAAGGTTAGAGAAGATTATTCGTTAGAGTGTGATCCGGCCGTTATTGGTAC
AGCTGTTAAAGGAAAGGAGGCGGTACACAGTGATCTAGGTTACTGGATTGAAAGTGAGAAGAATGATA
CATGGAGGCTAAAGAGGGCCCATCTAATCGAGATGAAAACGTGTGAATGGCCGAAGTCCCACACGTTG
TGGACAGATGGTATAGAAGAGTCGGATCTGATCATACCGAAGTCTTTAGCGGGGCCACTCAGTCATCA
CAATACGAGAGAGGGCTATAGGACCCAAATGAAAGGTCCATGGCACTCGGAAGAGCTTGAAATACGGT
TTGAGGAATGTCCAGGCACTAAAGTCCACGTGGAAGAAACATGTGGTACAAGAGGACCGTCTCTGAGA
TCGACCACTGCAAGTGGAAGGGTAATCGAGGAATGGTGTTGCAGGGAGTGCACGATGCCCCCACTATC
GTTCCGGGCGAAAGATGGCTGTTGGTATGGTATGGAGATACGTCCCAGGAAAGAACCGGAAAGCAACT
TAGTACGTTCAATGGTAACTGCA(NS2A)GGATCGACTGATCACATGGATCACTTCTCCCTTGGAGTA
CTTGTAATCCTGCTCATGGTACAGGAAGGGCTAAAGAAGAGAATGACGACAAAGATCATAATAAGCAC
ATCGATGGCAGTACTGGTAGCTATGATACTGGGAGGATTTTCGATGAGTGACCTAGCTAAGCTTGCGA
TTTTGATGGGTGCGACCTTCGCGGAAATGAATACTGGAGGAGATGTAGCGCATCTGGCGCTAATAGCG
GCATTTAAAGTCAGACCGGCGTTGCTGGTATCGTTCATCTTCCGTGCTAATTGGACGCCCCGTGAATC
GATGCTGCTGGCGTTGGCCTCGTGTCTATTGCAAACTGCGATATCCGCCTTGGAAGGTGACCTGATGG
TACTCATCAATGGTTTTGCGTTGGCCTGGTTAGCAATACGAGCGATGGTAGTTCCACGCACGGATAAC
ATCACGTTGGCAATCCTAGCTGCTCTGACGCCACTGGCCCGTGGCACACTGCTTGTAGCGTGGAGAGC
GGGCCTTGCTACGTGCGGGGGGTTTATGCTACTCTCTCTGAAAGGAAAAGGCAGTGTAAAGAAGAACT
TACCGTTTGTCATGGCGCTGGGACTAACGGCTGTAAGGCTGGTCGATCCCATCAACGTAGTAGGACTG
CTGTTACTAACAAGGAGTGGGAAACGG(NS2B)AGCTGGCCGCCTAGCGAAGTACTAACAGCTGTTGG
TCTGATATGCGCATTGGCGGGAGGGTTCGCGAAGGCAGATATAGAAATGGCTGGGCCGATGGCCGCGG
TAGGTCTGCTAATAGTCAGTTACGTAGTCTCAGGAAAAAGTGTGGACATGTATATTGAAAGAGCGGGT
GACATCACATGGGAAAAAGATGCGGAAGTAACTGGAAACAGTCCGCGGCTCGATGTAGCGCTAGATGA
AAGTGGTGATTTTTCCCTGGTAGAGGATGACGGTCCCCCCATGAGAGAAATCATACTCAAAGTAGTCC
TGATGACGATCTGTGGCATGAATCCGATAGCCATACCGTTTGCAGCTGGTGCGTGGTACGTATACGTA
AAGACTGGAAAACGT(NS3)AGTGGTGCTCTATGGGATGTACCTGCTCCCAAAGAAGTAAAAAAAGGG
GAGACCACGGATGGAGTATACAGAGTAATGACGCGTAGACTGCTAGGTTCGACACAAGTTGGTGTAGG
AGTTATGCAAGAAGGGGTCTTTCATACTATGTGGCATGTCACAAAAGGTTCCGCGCTGCGTAGCGGTG
AAGGTAGACTTGATCCGTACTGGGGAGATGTAAAGCAGGATCTAGTATCATACTGTGGTCCGTGGAAG
CTAGATGCGGCCTGGGACGGTCACAGCGAGGTACAGCTCTTGGCGGTACCCCCCGGAGAAAGAGCGAG
GAATATCCAGACTCTACCCGGAATATTTAAAACAAAGGATGGTGACATTGGAGCGGTAGCGCTGGATT
ATCCAGCAGGAACGTCAGGATCTCCGATCCTAGACAAATGTGGGAGAGTAATAGGACTTTATGGTAAT
GGGGTCGTAATCAAAAATGGTAGTTATGTTAGTGCGATCACCCAAGGTAGGAGGGAAGAAGAAACTCC
TGTTGAATGCTTCGAGCCGTCGATGCTGAAAAAGAAGCAGCTAACGGTCTTAGACTTACATCCTGGAG
CGGGGAAAACCCGAAGAGTTCTTCCGGAAATAGTCCGTGAAGCGATAAAAACACGTCTCCGTACTGTA
ATCTTAGCTCCGACCAGGGTTGTAGCTGCTGAAATGGAAGAGGCCCTTCGTGGGCTTCCAGTACGTTA
TATGACGACAGCAGTCAATGTAACCCACTCTGGTACAGAAATCGTTGACTTAATGTGTCATGCCACCT
TTACTTCACGTCTACTACAACCAATCAGAGTTCCCAACTATAATCTATATATTATGGATGAAGCCCAC
TTCACGGATCCCTCAAGTATAGCGGCAAGAGGATATATTTCAACAAGGGTTGAAATGGGCGAGGCGGC
GGCCATCTTCATGACGGCCACGCCACCGGGAACCCGTGATGCATTTCCGGATTCCAACTCACCGATTA
TGGACACGGAAGTGGAAGTTCCAGAGAGAGCGTGGAGCTCAGGTTTTGATTGGGTAACGGATCATTCG
GGAAAAACAGTTTGGTTTGTTCCGAGCGTGAGGAATGGCAATGAGATAGCAGCTTGTCTAACAAAGGC
TGGTAAACGGGTCATACAACTCAGCAGAAAAACTTTTGAAACAGAGTTCCAAAAAACAAAACATCAAG
AATGGGACTTTGTTGTTACAACTGACATATCAGAGATGGGTGCCAACTTTAAAGCTGACCGTGTCATA
GATTCGAGGAGATGCCTAAAGCCGGTCATACTAGATGGCGAGCGAGTCATTCTGGCGGGACCCATGCC
GGTCACACATGCGAGCGCTGCCCAAAGGAGGGGGCGTATAGGCAGGAATCCGAACAAACCTGGTGATG
AGTATCTATATGGAGGTGGTTGCGCAGAGACGGACGAAGACCATGCGCACTGGCTTGAAGCGAGAATG
CTCCTAGACAATATTTATCTCCAAGATGGTCTCATAGCCTCGCTATATCGACCTGAAGCCGACAAAGT
AGCGGCCATTGAGGGTGAGTTCAAGCTAAGGACGGAGCAACGTAAGACCTTTGTAGAACTCATGAAAA
GAGGTGATCTTCCTGTATGGCTGGCCTATCAAGTTGCATCTGCGGGAATAACCTATACAGATAGAAGA
TGGTGTTTTGATGGCACGACGAACAACACCATAATGGAAGATTCGGTGCCGGCAGAAGTGTGGACCAG
ACATGGAGAGAAACGTGTGCTCAAACCGAGGTGGATGGATGCCAGAGTTTGTTCAGATCATGCGGCGC
TGAAGTCATTTAAGGAGTTTGCGGCTGGGAAAAGA(NS4A)GGTGCGGCTTTTGGTGTAATGGAAGCC
CTGGGAACACTGCCGGGACACATGACGGAGAGATTCCAAGAAGCCATTGATAACCTCGCTGTACTCAT
GCGGGCGGAGACTGGAAGTAGGCCTTACAAAGCCGCGGCGGCGCAATTGCCGGAAACCCTAGAGACGA
TARTGCTTTTAGGGTTGCTGGGTACAGTCTCGCTAGGAATCTTCTTTGTCTTGATGCGTAACAAGGGC
ATAGGTAAGATGGGCTTTGGTATGGTGACTCTAGGGGCCAGCGCGTGGCTCATGTGGCTATCGGAAAT
TGAACCAGCCAGAATAGCATGTGTCCTAATTGTTGTATTCCTATTGCTGGTAGTACTCATACCTGAAC
CAGAAAAGCAACGTTCTCCCCAGGATAACCAAATGGCAATAATCATCATGGTAGCGGTAGGTCTTCTA
GGCTTGATTACGGCC(NS4B)AATGAACTAGGATGGTTGGAAAGAACAAAGTCGGACCTAAGCCATCT
AATGGGTAGGAGAGAGGAAGGGGCAACCATAGGTTTCTCAATGGATATTGACCTGCGTCCAGCCTCAG
CGTGGGCCATCTATGCGGCCTTGACAACGTTCATTACCCCGGCCGTCCAACATGCGGTGACCACCTCG
TACAACAACTATTCCTTAATGGCGATGGCGACGCAAGCTGGTGTGTTGTTTGGTATGGGTAAAGGGAT
GCCGTTCTACGCATGGGATTTTGGAGTCCCGCTACTAATGATAGGTTGTTACTCACAATTAACGCCCC
TGACCCTAATAGTAGCCATCATTTTACTCGTGGCGCATTACATGTACTTAATCCCAGGGCTACAGGCA
GCAGCGGCGCGTGCTGCGCAGAAGAGAACGGCGGCTGGCATCATGAAAAACCCTGTTGTAGATGGAAT
AGTAGTGACTGACATAGACACAATGACGATTGACCCCCAAGTAGAGAAAAAGATGGGTCAGGTGCTAC
TAATAGCAGTAGCGGTCTCCAGCGCGATACTGTCGCGGACCGCCTGGGGTTGGGGGGAGGCGGGGGCC
CTGATAACAGCCGCAACGTCCACTTTGTGGGAAGGTTCTCCGAACAAATACTGGAACTCGTCTACAGC
CACGTCACTGTGTAATATTTTTAGGGGTAGTTACTTGGCGGGAGCTTCTCTAATATACACAGTAACGA
GAAACGCTGGTTTGGTCAAGCGTCGT(NS5)GGGGGTGGTACAGGAGAGACGCTGGGAGAGAAATGGA
AAGCCCGCTTGAATCAGATGTCGGCGCTGGAGTTCTATTCCTACAAAAAATCAGGCATCACGGAGGTG
TGCCGTGAAGAGGCCCGTCGCGCCCTCAAAGACGGTGTGGCGACGGGAGGCCATGCGGTGTCCCGAGG
TAGTGCAAAGCTAAGATGGTTGGTAGAGCGGGGATATCTGCAGCCCTATGGTAAGGTCATTGATCTAG
GATGTGGCCGTGGGGGCTGGTCGTACTACGCCGCGACCATCCGCAAAGTACAAGAAGTGAAAGGTTAC
ACAAAAGGTGGCCCTGGTCATGAAGAACCCGTGTTGGTGCAAAGTTATGGGTGGAATATAGTCCGTCT
AAAGAGTGGGGTAGACGTCTTTCATATGGCGGCGGAGCCGTGTGATACGCTGCTGTGTGATATAGGTG
AGTCGTCATCTAGTCCGGAAGTGGAAGAAGCGCGGACGCTCCGTGTCCTCTCCATGGTAGGGGATTGG
CTAGAAAAAAGACCGGGAGCCTTTTGTATAAAAGTATTGTGCCCATATACCAGCACTATGATGGAAAC
GCTGGAGCGACTACAGCGTAGGTATGGTGGAGGACTGGTAAGAGTGCCACTATCCCGCAACTCGACAC
ATGAGATGTATTGGGTCTCTGGTGCGAAAAGCAATACCATAAAAAGTGTATCCACCACGTCGCAGCTC
CTCTTAGGGCGCATGGATGGGCCTAGACGTCCAGTGAAATATGAAGAGGATGTGAATCTAGGCTCTGG
CACGCGTGCTGTGGTAAGTTGCGCTGAAGCGCCCAACATGAAAATCATTGGTAATCGCATTGAAAGGA
TACGCAGTGAGCATGCGGAAACGTGGTTTTTTGACGAGAATCACCCATATAGGACGTGGGCTTACCAT
GGTAGCTATGAGGCGCCCACACAAGGTTCAGCGTCCTCGCTAATAAACGGTGTTGTCAGGCTACTGTC
AAAACCGTGGGATGTGGTAACTGGAGTCACGGGAATAGCCATGACGGACACCACACCGTATGGTCAAC
AAAGAGTTTTTAAGGAAAAAGTAGACACTAGGGTACCAGACCCCCAAGAAGGTACTCGTCAGGTAATG
AGCATGGTATCTTCCTGGTTATGGAAAGAGCTAGGTAAACACAAACGTCCACGAGTCTGTACGAAAGA
AGAGTTTATCAACAAGGTACGTAGCAATGCGGCATTAGGGGCGATATTTGAAGAAGAAAAAGAGTGGA
AAACTGCAGTGGAAGCGGTGAACGATCCGAGGTTCTGGGCGCTAGTGGACAAAGAAAGAGAGCATCAC
CTGAGAGGTGAGTGCCAGTCGTGTGTGTATAATATGATGGGAAAACGTGAAAAGAAACAAGGTGAATT
TGGAAAAGCCAAGGGCAGCCGTGCCATCTGGTATATGTGGCTAGGTGCTAGATTTCTAGAATTCGAAG
CCCTTGGATTCTTGAATGAGGATCACTGGATGGGTAGAGAGAACTCGGGAGGTGGTGTAGAAGGGCTG
GGTTTACAAAGACTAGGATATGTCCTAGAAGAAATGAGTCGTATACCGGGAGGAAGGATGTATGCGGA
TGACACTGCGGGCTGGGACACGCGCATTAGCCGTTTTGATCTGGAAAATGAAGCTCTAATAACCAACC
AAATGGAAAAAGGGCACAGGGCGTTGGCATTGGCGATAATCAAGTATACATACCAAAATAAAGTGGTA
AAAGTCCTTAGACCGGCTGAAAAAGGTAAAACAGTTATGGATATTATTTCGCGTCAAGACCAAAGGGG
TAGCGGACAAGTAGTCACTTACGCGCTTAACACATTTACGAACCTAGTGGTACAACTCATTCGTAATA
TGGAGGCGGAGGAAGTTCTAGAAATGCAAGACTTATGGCTGCTGCGTAGGTCAGAGAAAGTAACCAAC
TGGTTACAGAGCAACGGTTGGGATAGGCTAAAACGAATGGCGGTCAGTGGAGATGATTGTGTTGTGAA
GCCGATTGATGATCGTTTTGCACATGCGCTCAGGTTCTTAAATGATATGGGTAAAGTTAGAAAAGACA
CACAAGAATGGAAACCCTCGACTGGATGGGATAACTGGGAAGAAGTACCGTTTTGCTCGCACCACTTC
AATAAGCTCCATCTAAAGGACGGGCGTTCCATTGTGGTACCCTGCCGCCATCAAGATGAACTAATTGG
CCGGGCGCGCGTCTCTCCGGGGGCGGGATGGTCGATCCGGGAGACGGCTTGCCTAGCGAAATCATATG
CGCAAATGTGGCAGCTACTTTATTTCCATAGAAGGGACCTACGACTGATGGCGAATGCCATTTGTTCG
TCTGTGCCAGTAGACTGGGTTCCGACTGGGAGAACGACCTGGTCAATACATGGAAAGGGTGAATGGAT
GACGACTGAAGACATGCTAGTGGTGTGGAATAGAGTGTGGATAGAGGAGAACGATCACATGGAAGATA
AGACCCCAGTAACGAAATGGACGGACATTCCCTATTTAGGAAAAAGGGAAGATTTGTGGTGTGGTTCT
CTCATAGGTCACAGACCGCGTACCACCTGGGCGGAGAACATTAAAAATACAGTCAACATGGTACGCAG
GATCATAGGTGATGAAGAAAAATATATGGACTACCTATCGACCCAAGTTCGTTACTTGGGTGAAGAAG
GTTCTACACCTGGTGTGCTGTAA(NS5 end)
GCACCAATCTTAATGTTGTCAGGCCTGCTAGTCAGCCACAGCTTGGGGAAAGCTGTGCAGCCTGTGAC
CCCCCAGGAGAAGCTGGGAAACCAAGCCTATAGTCAGGCCGAGAACGCCATGGCACGGAAGAAGCCAT
GCTGCCTGTGAGCCCCTCAGAGGATACTGAGTCAAAAAACCCCACGCGCTTGGAGGCGCAGGATGGGA
AAAGAAGGTGGCGACCTTCCCCACCCTTCAATCTGGGGCCTGAACTGGAGATCAGCTGTGGATCCCCA
GAAGAGGGACTAGTGGTTAGAGGAGACCCCCCGGAAAACGCAAAACAGCATATTGACGCTGGGAAAGA
CCAGAGACTCCATGAGTTTCCACCACGCTGGCCGCCAGGCACAGATCGCCGAACTTCGGCGGCCGGTG
TGGGGAAATCCATGGTTTCT

SEQ ID NO:12. Vaccine candidate ZIKV-DO, more extensive sequence of flanking regions, with deoptimized region shown in underline, with locations of key regions indicated.

AGTTGTTGATCTGTGTGAATCAGACTGCGACAGTTCGAGTTTGAAGCGAAAGCTAGCAA
CAGTATCAACAGGTTTTATTTTGGATTTGGAAACGAGAGTTTCTGGTCATGAAAAACCCAAAAAAGAA
ATCCGGAGGATTCCGGATTGTCAATATGCTAAAACGCGGAGTAGCCCGTGTGAGCCCCTTTGGGGGCT
TGAAGAGGCTGCCAGCCGGACTTCTGCTGGGTCATGGGCCCATCAGGATGGTCTTGGCGATTCTAGCC
TTTTTGAGATTCACGGCAATCAAGCCATCACTGGGTCTCATCAATAGATGGGGTTCAGTTGGGAAAAA
AGAGGCTATGGAAATAATAAAGAAGTTCAAGAAAGATCTGGCTGCCATGCTGAGAATAATCAATGCTA
GGAAGGAGAAGAAGAGACGAGGCGCAGATACTAGTGTCGGAATTGTTGGCCTCCTGCTGACCACAGCT
ATGGCAGCGGAGGTCACTAGACGTGGGAGTGCATACTATATGTACTTGGACAGAAACGATGCTGGGGA
GGCCATATCTTTTCCAACCACATTGGGGATGAATAAGTGTTATATACAGATCATGGATCTTGGACACA
TGTGTGATGCCACCATGAGCTATGAATGCCCTATGCTGGATGAGGGGGTGGAACCAGATGACGTCGAT
TGTTGGTGCAACACGACGTCAACTTGGGTTGTGTACGGAACCTGCCATCACAAAAAAGGTGAAGCACG
GAGATCTAGAAGAGCTGTGACGCTCCCCTCCCATTCCACTAGGAAGCTGCAAACGCGGTCGCAAACCT
GGTTGGAATCAAGAGAATACACAAAGCACTTGATTAGAGTCGAAAATTGGATATTCAGGAACCCTGGC
TTCGCGTTAGCAGCAGCTGCCATCGCTTGGCTTTTGGGAAGCTCAACGAGCCAAAAAGTCATATACTT
GGTCATGATACTGCTGATTGCCCCGGCATACAGCATCAGGTGCATAGGAGTCAGCAATAGGGACTTTG
TGGAAGGTATGTCAGGTGGGACTTGGGTTGATGTTGTCTTGGAACATGGAGGTTGTGTCACCGTAATG
GCACAGGACAAACCGACTGTCGACATAGAGCTGGTTACAACAACAGTCAGCAACATGGCGGAGGTAAG
ATCCTACTGCTATGAGGCATCAATATCAGACATGGCTTCGGACAGCCGCTGCCCAACACAAGGTGAAG
CCTACCTTGACAAGCAATCAGACACTCAATATGTCTGCAAAAGAACGTTAGTGGACAGAGGCTGGGGA
AATGGATGTGGACTTTTTGGCAAAGGGAGCCTGGTGACATGCGCTAAGTTTGCATGCTCCAAGAAAAT
GACCGGGAAGAGCATCCAGCCAGAGAATCTGGAGTACCGGATAATGCTGTCAGTTCATGGCTCCCAGC
ACAGTGGGATGATCGTTAATGACACAGGACATGAAACTGATGAGAATAGAGCGAAAGTTGAGATAACG
CCCAATTCACCAAGAGCCGAAGCCACCCTGGGGGGTTTTGGAAGCCTAGGACTTGATTGTGAACCGAG
GACAGGCCTTGACTTTTCAGATTTGTATTACTTGACTATGAATAACAAGCACTGGTTGGTTCACAAGG
AGTGGTTCCACGACATTCCATTACCTTGGCACGCTGGGGCAGACACCGGAACTCCACACTGGAACAAC
AAAGAAGCACTGGTAGAGTTCAAGGACGCACATGCCAAAAGGCAAACTGTCGTGGTTCTAGGGAGTCA
AGAAGGAGCAGTTCACACGGCCCTTGCTGGAGCTCTGGAGGCTGAGATGGATGGTGCAAAGGGAAGGC
TGTCCTCTGGCCACTTGAAATGTCGCCTGAAAATGGATAAACTTAGATTGAAGGGCGTGTCATACTCC
TTGTGTACTGCAGCGTTCACATTCACCAAGATCCCGGCTGAAACACTGCACGGGACAGTCACAGTGGA
GGTACAGTACGCAGGGACAGATGGACCTTGCAAGGTTCCAGCTCAGATGGCGGTGGACATGCAAACTC
TGACCCCAGTTGGGAGGTTGATAACCGCTAACCCCGTAATCACTGAAAGCACTGAGAACTCTAAGATG
ATGCTGGAACTTGATCCACCATTTGGGGACTCTTACATTGTCATAGGAGTCGGGGAGAAGAAGATCAC
CCACCACTGGCACAGGAGTGGCAGCACCATTGGAAAAGCATTTGAAGCCACTGTGAGAGGTGCCAAGA
GAATGGCAGTCTTGGGAGACACAGCCTGGGACTTTGGATCAGTTGGAGGCGCTCTCAACTCATTGGGC
AAGGGCATCCATCAAATTTTTGGAGCAGCTTTCAAATCATTGTTTGGAGGAATGTCCTGGTTCTCACA
AATTCTCATTGGAACGTTGCTGATGTGGTTGGGTCTGAACACAAAGAATGGATCTATTTCCCTTATGT
GCTTGGCCTTAGGGGGGGTGTTGATCTTCTTATCCACAGCCGTCTCTGCTGAT(NS1)GTCGGTTGTT
CGGTAGATTTTTCGAAAAAAGAAACGCGATGTGGTACGGGTGTATTTGTATATAATGACGTAGAAGCG
TGGCGAGACCGATACAAGTATCATCCGGACTCGCCGCGACGATTAGCGGCGGCGGTAAAACAAGCGTG
GGAAGACGGTATATGCGGTATATCGTCGGTATCGCGAATGGAAAATATAATGTGGCGATCGGTAGAAG
GTGAGTTAAATGCGATACTAGAAGAGAATGGCGTACAACTAACGGTAGTAGTCGGCTCGGTAAAAAAT
CCCATGTGGCGAGGTCCGCAGCGATTGCCCGTCCCCGTCAATGAGCTACCCCATGGTTGGAAGGCGTG
GGGTAAATCGTACTTCGTACGAGCGGCGAAGACGAATAATTCCTTTGTAGTCGATGGTGACACGCTAA
AGGAATGCCCGTTAAAACATCGAGCGTGGAATTCCTTTTTGGTCGAGGATCATGGTTTCGGTGTATTC
CATACCAGTGTATGGTTAAAGGTACGAGAAGATTATTCGTTAGAGTGTGATCCGGCCGTAATTGGCAC
GGCGGTAAAGGGCAAGGAGGCGGTACATAGTGATCTCGGTTACTGGATTGAGAGTGAGAAGAATGACA
CGTGGCGCCTAAAGCGCGCCCATCTAATAGAGATGAAAACGTGTGAATGGCCGAAGTCGCACACGTTG
TGGACGGATGGCATAGAAGAGAGTGATCTAATAATACCCAAGTCGTTAGCGGGTCCGTTATCCCATCA
TAATACCCGAGAGGGTTACCGCACCCAAATGAAAGGTCCGTGGCATAGTGAAGAGTTGGAAATTCGGT
TCGAGGAATGTCCGGGTACCAAGGTACACGTCGAGGAAACGTGTGGCACGCGAGGCCCGTCGCTACGA
TCGACCACCGCGTCCGGCCGCGTCATAGAGGAATGGTGCTGCCGCGAGTGCACGATGCCCCCGCTATC
GTTCCGGGCGAAAGATGGTTGTTGGTATGGAATGGAGATACGCCCCCGCAAAGAACCGGAATCCAATT
TAGTACGCTCGATGGTCACCGCGGGCTCGACCGATCATATGGACCATTTCTCGTTG(NS2A)GGCGTC
TTGGTCATACTATTAATGGTCCAAGAAGGTCTAAAGAAGCGAATGACCACGAAGATAATAATATCCAC
GTCGATGGCGGTCCTAGTAGCGATGATACTAGGCGGCTTTTCGATGAGTGACCTAGCGAAGTTGGCGA
TTTTGATGGGTGCCACCTTCGCGGAAATGAATACCGGCGGCGATGTAGCGCATCTAGCGCTAATAGCG
GCGTTCAAAGTACGACCGGCGTTGCTAGTATCGTTCATATTCCGAGCGAATTGGACGCCCCGTGAATC
CATGCTACTAGCCTTGGCCTCGTGTTTGTTGCAAACCGCGATATCGGCCTTGGAAGGTGACCTAATGG
TATTAATAAATGGTTTCGCGTTGGCCTGGTTGGCGATACGAGCGATGGTAGTACCGCGCACCGATAAT
ATAACCTTGGCGATACTAGCGGCGCTAACGCCGCTAGCCCGGGGTACGCTATTGGTCGCGTGGCGAGC
GGGTTTGGCGACCTGCGGTGGTTTTATGTTATTATCGCTAAAGGGCAAAGGTAGTGTCAAGAAGAATT
TACCGTTTGTAATGGCCCTAGGCCTCACCGCGGTCCGCCTAGTAGACCCCATAAATGTCGTCGGCCTA
CTATTATTAACGCGCAGTGGTAAGCGGTCCTGGCCC(NS2B)CCCTCCGAAGTATTAACGGCGGTAGG
TCTAATATGCGCGTTGGCGGGCGGTTTCGCCAAGGCGGATATAGAGATGGCGGGTCCCATGGCCGCGG
TAGGTCTACTCATTGTAAGTTACGTCGTATCGGGCAAGAGTGTCGACATGTACATTGAACGAGCGGGT
GACATAACGTGGGAAAAAGATGCGGAAGTAACCGGCAATAGTCCCCGGTTAGATGTCGCGCTCGATGA
GAGTGGTGATTTCTCGCTAGTCGAGGATGACGGTCCCCCGATGCGAGAGATAATATTAAAGGTCGTAC
TAATGACCATATGTGGTATGAATCCGATAGCCATACCCTTCGCGGCGGGCGCGTGGTACGTATACGTC
AAGACCGGCAAACGC(NS3)AGTGGTGCGCTCTGGGATGTCCCCGCGCCCAAGGAAGTAAAAAAGGGT
GAGACCACGGATGGCGTCTACCGAGTAATGACCCGTCGACTACTCGGTTCGACGCAAGTAGGCGTCGG
CGTAATGCAAGAGGGTGTATTCCACACCATGTGGCATGTAACGAAAGGCTCGGCGCTACGATCCGGTG
AAGGTCGATTGGATCCGTACTGGGGCGATGTAAAGCAAGATCTAGTCTCGTACTGTGGTCCGTGGAAG
CTCGATGCCGCCTGGGACGGTCACTCCGAGGTCCAGTTATTGGCCGTCCCGCCGGGCGAGCGAGCGCG
CAATATACAAACTCTACCCGGCATATTCAAGACGAAGGATGGTGACATTGGCGCGGTAGCGCTAGATT
ACCCGGCGGGCACTTCGGGCTCGCCGATACTCGACAAGTGTGGTCGAGTCATAGGCTTGTATGGTAAT
GGTGTAGTCATAAAAAATGGTAGTTATGTAAGTGCCATAACCCAAGGTCGCCGCGAAGAAGAGACCCC
CGTAGAGTGCTTCGAGCCCTCGATGCTAAAGAAGAAGCAACTCACTGTATTAGACTTGCATCCCGGCG
CGGGTAAAACCCGCCGAGTATTGCCCGAAATAGTACGTGAAGCCATAAAAACGCGATTACGTACCGTC
ATATTAGCGCCGACCCGCGTAGTAGCGGCGGAAATGGAGGAGGCCTTGCGAGGTTTGCCGGTCCGTTA
TATGACGACGGCGGTAAATGTAACCCATTCGGGCACGGAAATAGTAGACTTAATGTGCCATGCCACCT
TCACCTCGCGTCTCCTCCAGCCGATACGAGTACCCAATTATAATCTATATATTATGGATGAGGCCCAT
TTCACGGATCCCTCGAGTATAGCGGCGCGAGGCTACATTTCGACGCGCGTAGAGATGGGTGAGGCGGC
GGCCATATTCATGACCGCCACGCCGCCGGGCACCCGTGACGCGTTCCCGGACTCGAATTCGCCGATTA
TGGACACCGAAGTCGAAGTACCGGAGCGAGCCTGGTCCTCGGGTTTTGATTGGGTCACGGATCATTCG
GGCAAAACGGTATGGTTCGTACCGTCCGTCCGCAATGGTAATGAGATAGCGGCGTGTCTAACGAAGGC
GGGCAAACGGGTAATACAGTTATCCCGAAAGACCTTCGAGACGGAGTTCCAAAAAACGAAACATCAAG
AGTGGGACTTCGTAGTCACGACCGACATTTCGGAGATGGGTGCCAATTTCAAAGCGGACCGTGTAATA
GATTCGCGCCGATGCCTCAAGCCGGTAATATTGGATGGTGAGCGAGTAATTCTAGCGGGCCCCATGCC
CGTAACGCATGCCTCCGCGGCCCAACGCCGCGGTCGCATAGGTCGCAATCCCAATAAACCCGGCGATG
AGTATCTATATGGCGGTGGTTGCGCGGAGACCGACGAAGACCATGCGCATTGGTTGGAAGCGCGAATG
TTATTGGACAATATTTACTTACAAGATGGTTTAATAGCCTCGTTATATCGACCCGAGGCCGACAAAGT
AGCGGCCATTGAGGGCGAGTTCAAGTTGCGCACGGAGCAACGCAAGACCTTCGTCGAATTAATGAAAC
GAGGCGATTTGCCCGTATGGCTAGCCTATCAAGTAGCGTCGGCAGGTATAACCTACACGGATCGACGA
TGGTGCTTCGATGGTACGACCAATAATACCATAATGGAAGATAGTGTGCCGGCAGAGGTGTGGACCAG
ACACGGAGAGAAAAGAGTGCTCAAACCGAGGTGGATGGACGCCAGAGTTTGTTCAGATCATGCGGCCC
TGAAGTCATTCAAGGAGTTTGCCGCTGGGAAAAGA(NS4A)GGAGCGGCTTTTGGAGTGATGGAAGCC
CTGGGAACACTGCCAGGACACATGACAGAGAGATTCCAGGAAGCCATTGACAACCTCGCTGTGCTCAT
GCGGGCAGAGACTGGAAGCAGGCCTTACAAAGCCGCGGCGGCCCAATTGCCGGAGACCCTAGAGACCA
TAATGCTTTTGGGGTTGCTGGGAACAGTCTCGCTGGGAATCTTCTTCGTCTTGATGAGGAACAAGGGC
ATAGGGAAGATGGGCTTTGGAATGGTGACTCTTGGGGCCAGCGCATGGCTCATGTGGCTCTCGGAAAT
TGAGCCAGCCAGAATTGCATGTGTCCTCATTGTTGTGTTCCTATTGCTGGTGGTGCTCATACCTGAGC
CAGAAAAGCAAAGATCTCCCCAGGACAACCAAATGGCAATCATCATCATGGTAGCAGTAGGTCTTCTG
GGCTTGATTACCGCC(NS4B)AATGAACTCGGATGGTTGGAGAGAACAAAGAGTGACCTAAGCCATCT
AATGGGAAGGAGAGAGGAGGGGGCAACCATAGGATTCTCAATGGACATTGACCTGCGGCCAGCCTCAG
CTTGGGCCATCTATGCTGCCTTGACAACTTTCATTACCCCAGCCGTCCAACATGCAGTGACCACCTCA
TACAACAACTACTCCTTAATGGCGATGGCCACGCAAGCTGGAGTGTTGTTTGGTATGGGCAAAGGGAT
GCCATTCTACGCATGGGACTTTGGAGTCCCGCTGCTAATGATAGGTTGCTACTCACAATTAACACCCC
TGACCCTAATAGTGGCCATCATTTTGCTCGTGGCGCACTACATGTACTTGATCCCAGGGCTGCAGGCA
GCAGCTGCGCGTGCTGCCCAGAAGAGAACGGCAGCTGGCATCATGAAGAACCCTGTTGTGGATGGAAT
AGTGGTGACTGACATTGACACAATGACAATTGACCCCCAAGTGGAGAAAAAGATGGGACAGGTGCTAC
TCATAGCAGTAGCCGTCTCCAGCGCCATACTGTCGCGGACCGCCTGGGGGTGGGGGGAGGCTGGGGCC
CTGATCACAGCCGCAACTTCCACTTTGTGGGAAGGCTCTCCGAACAAGTACTGGAACTCCTCTACAGC
CACTTCACTGTGTAACATTTTTAGGGGAAGTTACTTGGCTGGAGCTTCTCTAATCTACACAGTAACAA
GAAACGCTGGCTTGGTCAAGAGACGT(NS5)GGGGGTGGAACAGGAGAGACCCTGGGAGAGAAATGGA
AGGCCCGCTTGAACCAGATGTCGGCCCTGGAGTTCTACTCCTACAAAAAGTCAGGCATCACCGAGGTG
TGCAGAGAAGAGGCCCGCCGCGCCCTCAAGGACGGTGTGGCAACGGGAGGCCATGCTGTGTCCCGAGG
AAGTGCAAAGCTGAGATGGTTGGTGGAGCGGGGATACCTGCAGCCCTATGGAAAGGTCATTGATCTTG
GATGTGGCAGAGGGGGCTGGAGTTACTACGCCGCCACCATCCGCAAAGTTCAAGAAGTGAAAGGATAC
ACAAAAGGAGGCCCTGGTCATGAAGAACCCGTGTTGGTGCAAAGCTATGGGTGGAACATAGTCCGTCT
TAAGAGTGGGGTGGACGTCTTTCATATGGCGGCTGAGCCGTGTGACACGCTGCTGTGTGACATAGGTG
AGTCATCATCTAGTCCTGAAGTGGAAGAAGCACGGACGCTCAGAGTCCTCTCCATGGTGGGGGATTGG
CTTGAAAAAAGACCAGGAGCCTTTTGTATAAAAGTGTTGTGCCCATACACCAGCACTATGATGGAAAC
CCTGGAGCGACTGCAGCGTAGGTATGGGGGAGGACTGGTCAGAGTGCCACTCTCCCGCAACTCTACAC
ATGAGATGTACTGGGTCTCTGGAGCGAAAAGCAACACCATAAAAAGTGTGTCCACCACGAGCCAGCTC
CTCTTGGGGCGCATGGACGGGCCTAGAAGGCCAGTGAAATATGAGGAGGATGTGAATCTCGGCTCTGG
CACGCGGGCTGTGGTAAGCTGCGCTGAAGCTCCCAACATGAAGATCATTGGTAACCGCATTGAAAGGA
TCCGCAGTGAGCACGCGGAAACGTGGTTCTTTGACGAGAACCACCCATATAGGACATGGGCTTACCAT
GGAAGCTATGAGGCCCCCACACAAGGGTCAGCGTCCTCTCTAATAAACGGGGTTGTCAGGCTCCTGTC
AAAACCCTGGGATGTGGTGACTGGAGTCACAGGAATAGCCATGACCGACACCACACCGTATGGTCAGC
AAAGAGTTTTCAAGGAAAAAGTGGACACTAGGGTGCCAGACCCCCAAGAAGGCACTCGTCAGGTTATG
AGCATGGTCTCTTCCTGGTTGTGGAAAGAGCTAGGCAAACACAAACGGCCACGAGTCTGTACCAAAGA
AGAGTTCATCAACAAGGTTCGTAGCAATGCAGCATTAGGGGCAATATTTGAAGAGGAAAAAGAGTGGA
AGACTGCAGTGGAAGCTGTGAACGATCCAAGGTTCTGGGCTCTAGTGGACAAGGAAAGAGAGCACCAC
CTGAGAGGAGAGTGCCAGAGTTGTGTGTATAACATGATGGGAAAAAGAGAAAAGAAACAAGGGGAATT
TGGAAAGGCCAAGGGCAGCCGCGCCATCTGGTATATGTGGCTAGGGGCTAGATTTCTAGAGTTCGAAG
CCCTTGGATTCTTGAACGAGGATCACTGGATGGGGAGAGAGAACTCAGGAGGTGGTGTTGAAGGGCTG
GGATTACAAAGACTCGGATATGTCCTAGAAGAGATGAGTCGTATACCAGGAGGAAGGATGTATGCAGA
TGACACTGCTGGCTGGGACACCCGCATTAGCAGGTTTGATCTGGAGAATGAAGCTCTAATCACCAACC
AAATGGAGAAAGGGCACAGGGCCTTGGCATTGGCCATAATCAAGTACACATACCAAAACAAAGTGGTA
AAGGTCCTTAGACCAGCTGAAAAAGGGAAAACAGTTATGGACATTATTTCGAGACAAGACCAAAGGGG
GAGCGGACAAGTTGTCACTTACGCTCTTAACACATTTACCAACCTAGTGGTGCAACTCATTCGGAATA
TGGAGGCTGAGGAAGTTCTAGAGATGCAAGACTTGTGGCTGCTGCGGAGGTCAGAGAAAGTGACCAAC
TGGTTGCAGAGCAACGGATGGGATAGGCTCAAACGAATGGCAGTCAGTGGAGATGATTGCGTTGTGAA
GCCAATTGATGATAGGTTTGCACATGCCCTCAGGTTCTTGAATGATATGGGAAAAGTTAGAAAGGACA
CACAAGAGTGGAAACCCTCAACTGGATGGGACAACTGGGAAGAAGTTCCGTTTTGCTCCCACCACTTC
AACAAGCTCCATCTCAAGGACGGGAGGTCCATTGTGGTTCCCTGCCGCCACCAAGATGAACTGATTGG
CCGGGCCCGCGTCTCTCCAGGGGCGGGATGGAGCATCCGGGAGACTGCTTGCCTAGCAAAATCATATG
CGCAGATGTGGCAGCTCCTTTATTTCCACAGAAGGGACCTCCGACTGATGGCCAATGCCATTTGTTCA
TCTGTGCCAGTTGACTGGGTTCCAACTGGGAGAACTACCTGGTCAATCCATGGAAAGGGAGAATGGAT
GACCACTGAAGACATGCTTGTGGTGTGGAACAGAGTGTGGATTGAGGAGAACGACCACATGGAAGACA
AGACCCCAGTTACGAAATGGACAGACATTCCCTATTTGGGAAAAAGGGAAGACTTGTGGTGTGGATCT
CTCATAGGGCACAGACCGCGCACCACCTGGGCTGAGAACATTAAAAACACAGTCAACATGGTGCGCAG
GATCATAGGTGATGAAGAAAAGTACATGGACTACCTATCCACCCAAGTTCGCTACTTGGGTGAAGAAG
GGTCTACACCTGGAGTGCTGTAA(NS5 end)
GCACCAATCTTAATGTTGTCAGGCCTGCTAGTCAGCCACAGCTTGGGGAAAGCTGTGCAGCCTGTGAC
CCCCCAGGAGAAGCTGGGAAACCAAGCCTATAGTCAGGCCGAGAACGCCATGGCACGGAAGAAGCCAT
GCTGCCTGTGAGCCCCTCAGAGGATACTGAGTCAAAAAACCCCACGCGCTTGGAGGCGCAGGATGGGA
AAAGAAGGTGGCGACCTTCCCCACCCTTCAATCTGGGGCCTGAACTGGAGATCAGCTGTGGATCCCCA
GAAGAGGGACTAGTGGTTAGAGGAGACCCCCCGGAAAACGCAAAACAGCATATTGACGCTGGGAAAGA
CCAGAGACTCCATGAGTTTCCACCACGCTGGCCGCCAGGCACAGATCGCCGAACTTCGGCGGCCGGTG
TGGGGAAATCCATGGTTTCT

Preferred features, embodiments and variations of the invention may be discerned from the following Detailed Description of the invention.

### DETAILED DESCRIPTION

The present inventors have primarily developed a vaccine comprising live-attenuated Zika virus comprising a (partly) codon deoptimized Zika viral genome. Using codon deoptimization (CD) technology, the inventors inserted a number of codon changes in the genome of the virus (wild-type Zika virus), with the objective of decreasing replication efficiency in mammalian cells and rendering the virus attenuated compared to wild-type ZIKV. Using this strategy, some resulting viruses were strongly attenuated but still produced viral proteins to a level comparable to wild-type virus. Thus, using codon deoptimization technology, the inventors were able to generate live attenuated ZIKV vaccine candidates.

By inserting a substantial number of changes into each vaccine candidate, the chance of reversion to wild-type is negligible, which is a crucial safety feature of the vaccines. This represents a substantial competitive advantage over vaccines with only a small number of mutations. To the best of the inventors' knowledge, no other ZIKV vaccines have been generated using codon deoptimization technology.

Codon deoptimization in case of Zika virus presumably results in slower polyprotein translation leading to slower replication and, as a result, in attenuation of the virus, compared with wild-type Zika virus. Such vaccine candidates have virtually no risk of deattenuation (the chance of reversion to wild-type is negligible) because of too many substitutions, all of which have, taken alone, minimal effect on virus, have been made in the coding sequence.

‘Codon deoptimization’ (CD), as used herein, involves replacing normal codons in the wild-type Zika virus genome with synonymous codons so that the resulting virus proteins are identical to wild-type virus proteins. Moreover, the resulting virus is highly attenuated, but protein function is not compromised.

By ‘live attenuated’ it is meant that the virus demonstrates substantially reduced or preferably no clinical signs of disease when administered to a subject, compared with wild-type Zika virus.

In some embodiments codon deoptimization results in no less than about 200 codon changes in the viral genome. In some embodiments codon deoptimization results in no more than about 800 codon changes in the viral genome (with the upper limit for substitution being where the virus does not usually grow at all). In some embodiments codon deoptimization results in between about 200 and about 800 codon changes in the viral genome. This 200 to 800 codon change range includes all integers between 200 and 800, including 201, 202 . . . 798 and 799 codon changes. In some embodiments codon deoptimization results in a minimum of about 286 codon changes in the viral genome. In some embodiments codon deoptimization results in a maximum of about 651 codon changes in the viral genome. In some embodiments codon deoptimization results in between about 286 and 651 codon changes in the viral genome. This range includes all integers between 286 and 651, including 287 . . . 650 codon changes. In some embodiments some or all of the codon changes can be situated immediately next to one another, in sequence. In some embodiments some or all of the codon changes can be spaced apart from each other such that they are not situated immediately next to one another, in sequence—E.g. 3 to 4 codon (triplet) spacings. In some embodiments some of the codon changes can be spaced apart from each other and some of the codon changes can be situated immediately next to one another.

In some embodiments codon deoptimization occurs in no less than about a 1700 nucleotide region of the genome. The region can be continuous/contiguous or not. In some embodiments codon deoptimization occurs no more than in about a 7900 nucleotide region of the genome. The region can be continuous/contiguous or not. In some embodiments codon deoptimization occurs in a continuous genome region with a length of about 1800 to about 3600 nucleotides. In some embodiments codon deoptimization results in no less than about an 1800 nucleotide region of the genome, with no less than about 250 codon changes within that nucleotide region. In some embodiments codon deoptimization results in no more than about a 7900 nucleotide region of the genome, with no more than about 800 codon changes within that nucleotide region. In some embodiments about 20-60% of the coding region of the genome is codon deoptimized, preferably 18-36% of the genome, compared to wild-type ZIKV.

In some embodiments the non-structural region of the viral genome is codon deoptimized. In some embodiments only the non-structural region of the viral genome is codon deoptimized. In some embodiments any one or more of the genes NS1, 2A, NS2B, NS3, NS4A, NS4B and NS5 are codon deoptimized. In some embodiments any contiguous genome region from the NS1 to NS5 region corresponding to at least 600 amino acid residues of viral polyprotein is codon deoptimized. In some embodiments the genes NS1, 2A, NS2B, NS3, NS4A, NS4B and NS5 are codon deoptimized. In some embodiments every 3^{rd} or 4^{th} codon is deoptimized along the entire nonstructural ZIKV coding region. In some embodiments the genes NS1, 2A, NS2B and NS3 are codon deoptimized. In some embodiments approximately 700 base changes are made. In some embodiments the gene NS3 is codon deoptimized. In some embodiments about 350 changes base changes are made. In some embodiments approximately 700 codon substitutions are made along the entire nonstructural ZIKV coding region.

In some embodiments the codon deoptimization results in slower polyprotein translation leading to slower replication and, as a result, in attenuation of the virus. In some embodiments every codon in the wild-type Zika virus genome or region thereof was analyzed in terms of its usage frequency in Homo sapiens, and if the codon was frequent then it was changed in the viral genome to a least frequently used synonymous codon. In some embodiments a codon for an amino acid with codon degeneracy was changed only if the synonymous codons for that amino acid occurred in significantly different frequencies of usage in the genome of Homo sapiens. In some embodiments Asp, and Asn codons of the viral genome are left unchanged. In some embodiments a codon for an amino acid with high codon degeneracy was changed to a synonymous codon that was used least frequently or rarely in the genome of Homo sapiens. In some embodiments a viral region most rich in codons that can be substituted for rare codon variants is codon deoptimized. In some embodiments Leu codons of the viral genome are changed. In some embodiments Leu codons of the viral genome are changed to the rare CUA codon. In some embodiments the viral genome prior to codon deoptimization has a very similar nucleotide sequence to a Zika strain associated with microcephaly. In some embodiments the wild-type Zika viral genome is that of Brazilian Zika virus (ZIKV) strain BeH819016. In some embodiments the chance of deattenuation to wild-type Zika is negligible.

Preferably the codon deoptimized Zika viral genome is generated using codon deoptimization technology.

In some embodiments the codon deoptimized genome has the deoptimized codons of vaccine candidate ZIKV-DO-NS3 as shown in the NS3 region of SEQ ID NO:3, 4, 5 or 10. In some embodiments the codon deoptimized genome can have about 200 or more of the codon changes of vaccine candidate ZIKV-DO-NS3 shown in SEQ ID NO:3, 4, 5 or 10, including all integers between about 200 and about 350, including 201, 202 . . . 348 and 349 codon changes.

In some embodiments the codon deoptimized genome has the deoptimized codons of vaccine candidate ZIKV-DO-scattered as shown in SEQ ID NO:6, 7 or 11. In some embodiments the codon deoptimized genome can have about 200 or more of the codon changes of vaccine candidate ZIKV-DO-scattered shown in SEQ ID NO: 6, 7 or 11, including all integers between about 200 and about 700, including 201, 202 . . . 698 and 699 codon changes.

In some embodiments the codon deoptimized genome has the deoptimized codons of vaccine candidate ZIKV-DO as shown in SEQ ID NO:8, 9 or 12. In some embodiments the codon deoptimized genome can have about 200 or more of the codon changes of vaccine candidate ZIKV-DO-scattered shown in SEQ ID NO: 8, 9 or 12, including all integers between about 200 and about 700, including 201, 202 . . . 698 and 699 codon changes.

In some embodiments the codon deoptimized genome has the deoptimized codons of the nonstructural region of SEQ ID NO:1 as shown in FIG. 1b. In some embodiments the codon deoptimized genome can have about 1 or more of the codon changes of SEQ ID NO:1, including all integers between about 1 and about 72, including 2, 3 . . . 70 and 71 codon changes.

The live attenuated recombinant Zika virus, recombinant Zika virus, recombinant Zika virus particle or recombinant Zika virus nucleic acid comprising a partly codon deoptimized Zika viral genome can be of any suitable form and can be prepared in any suitable way. Likewise, the recombinant, isolated or substantially purified nucleic acid comprising a partly codon deoptimized Zika viral genome or partly codon deoptimized region thereof can be prepared in any suitable way. Such techniques are described elsewhere in this specification (eg. see below), the entire contents of which are incorporated herein by way of cross-reference.

Likewise, a vaccine, pharmaceutical preparation or immunogenic composition comprising the above can be of any suitable form and can be prepared in any suitable way. Such techniques are described elsewhere in this specification, the entire contents of which are incorporated herein by way of cross-reference.

In addition to a live attenuated recombinant Zika virus vaccine, pharmaceutical preparation or immunogenic composition, the present invention encompasses recombinant Zika virus particles, nucleic acid and genetic vaccines that comprise a partly codon deoptimized Zika viral genome in the form of a nucleic acid. The nucleic acid can be DNA or RNA that is self-replicating/self-amplifying once used for vaccination. The nucleic acid can relate to the Zika viral genome or Zika viral anti-genome. Such techniques are described in the following references, the entire contents of which are incorporated herein by way of cross-reference: Karl Ljungberg & Peter Liljeström (2015) Self-replicating alphavirus RNA vaccines, Expert Review of Vaccines, 14:2, 177-194, DOI: 10.1586/14760584.2015.965690; Rodriguez-Gascón A, del Pozo-Rodrlguez A, Solinis M A (2014) Development of nucleic acid vaccines: use of self-amplifying RNA in lipid nanoparticles. Int J Nanomedicine. 9: 1833-1843; US 2014/0112979 A1.

The vaccine, pharmaceutical preparation or immunogenic composition can comprise live virus or inactivated virus, provided that it is self-replicating/self-amplifying after vaccination. If inactivated, it can be inactivated in any suitable way (e.g. using high or low temperatures, or chemically).

The vaccine, pharmaceutical preparation or immunogenic composition can comprise a delivery system or carrier or aid, and these can be of any suitable form and can be prepared in any suitable way. Suitable examples include a plasmid or vector to assist with self-replication/self-amplification, an RNA nanocarrier for RNA delivery, and lipid-based formulations for delivery, including liposomes, nanoemulsions and solid lipid nanoparticles.

In some embodiments the vaccine can be prepared by way of passing recombinant ZIKV through a filter, such as a 0.22 μm hydrophilic PVDF membrane or hydrophilic Polyethersulfone membrane.

In some embodiments the vaccine can be stored long term and remain viable at a temperature of between about −20° C. and about −80° C. By “long-term” it is meant that the vaccine can remain viable for at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 days. In some embodiments it is possible that the vaccine can remain viable for more than 60 days.

The live attenuated virus can be in the form of an isolate. The isolate may comprise cells, such as mammalian, insect (e.g. mosquito) or other types of cells.

The method of preventing the subject from contracting a viral infection, treating a subject having a viral infection, or reducing the severity of a viral infection, can be carried out in any suitable way.

The vaccine, live attenuated virus, pharmaceutical preparation and immunogenic composition (described hereafter as “the compositions”) can be administered independently, either systemically or locally, by any method standard in the art, for example, subcutaneously, intravenously, parenterally, intraperitoneally, intradermally, intramuscularly, topically, or nasally.

The compositions can comprise conventional non-toxic, physiologically or pharmaceutically acceptable ingredients or vehicles suitable for the method of administration and are well known to an individual having ordinary skill in this art. The compositions can, for example, comprise an adjuvant. The adjuvant can be, for example, an aluminium salt (e.g. aluminium hydroxide), monophosphoryl lipid A, or, emulsion of water and oil (e.g. MF59). The term “pharmaceutically acceptable carrier” as used herein is intended to include diluents such as saline and aqueous buffer solutions. The compositions can be in aqueous or lyophilized form.

A variety of devices are known in the art for delivery of the compositions including, but not limited to, syringe and needle injection, bifurcated needle administration, administration by intradermal patches or pumps, intradermal needle-free jet delivery (intradermal etc), intradermal particle delivery, or aerosol powder delivery.

The compositions can be administered independently one or more times to achieve, maintain or improve upon a desired effect/result. It is well within the skill of an artisan to determine dosage or whether a suitable dosage of the composition comprises a single administered dose or multiple administered doses. An appropriate dosage depends on the subject's health, the induction of immune response and/or prevention of infection caused by the alphavirus, the route of administration and the formulation used. For example, a therapeutically active amount of the compound may vary according to factors such as the disease state, age, sex, and weight of the subject, and the ability of the composition to elicit a desired response in the subject. Dosage regime may be adjusted to provide the optimum therapeutic response. For example, a subject may be administered a ‘booster’ vaccination one or two weeks following the initial administration.

The vector can also be prepared in any suitable way.

The cell (insect, mammalian or other) or isolate comprising the vector or virus can be prepared in any suitable way.

Suitable protocols for carrying out one or more of the above-mentioned techniques can be found in “Current Protocols in Molecular Biology”, July 2008, JOHN WILEY AND SONS; D. M. WEIR ANDCC BLACKWELL, “Handbook Of Experimental Immunology”, vol. I-IV, 1986; JOHN E. COLIGAN, ADA M. KRUISBEEK, DAVID H. MARGULIES, ETHAN M. SHEVACH, WARREN STROBER, “Current Protocols in Immunology”, 2001, JOHN WILEY & SONS; “Immunochemical Methods In Cell And Molecular Biology”, 1987, ACADEMIC PRESS; SAMBROOK ET AL., “Molecular Cloning: A Laboratory Manual, 3d ed.,”, 2001, COLD SPRING HARBOR LABORATORY PRESS; “Vaccine Design, Methods and Protocols”, Volume 2, Vaccines for Veterinary Diseases, Sunil Thomas in Methods in Molecular Biology (2016); and, “Vaccine Design, Methods and Protocols”, Volume 1: Vaccines for Human Diseases, Sunil Thomas in Methods in Molecular Biology (2016), the entire contents of which are incorporated herein by way of cross-reference.

Any suitable type of subject can be used. The subject can be any suitable mammal. Mammals include humans, primates, livestock and farm animals (e.g. horses, sheep and pigs), companion animals (e.g. dogs and cats), and laboratory test animals (e.g. rats, mice and rabbits). The subject is preferably human.

‘Nucleic acid’ as used herein includes ‘polynucleotide’, ‘oligonucleotide’, and ‘nucleic acid molecule’, and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide.

As used herein, the term ‘recombinant’ refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The terms ‘isolated’ or ‘purified’ as used herein mean essentially free of association with other biological components/contaminants, e.g., as a naturally occurring protein that has been separated from cellular and other contaminants by the use of antibodies or other methods or as a purification product of a recombinant host cell culture.

Preferred embodiments of the invention are defined in the following numbered paragraphs:

1. Live attenuated recombinant Zika virus, recombinant Zika virus, recombinant Zika virus particle or recombinant Zika virus nucleic acid comprising a partly codon deoptimized Zika viral genome.

2. A recombinant, isolated or substantially purified nucleic acid comprising a partly codon deoptimized Zika viral genome or partly codon deoptimized region thereof.

3. A vector containing the nucleic acid of paragraph 2.

4. A cell or isolate containing the live attenuated recombinant Zika virus, recombinant Zika virus, recombinant Zika virus particle or recombinant Zika virus nucleic acid of paragraph 1, the nucleic acid of the paragraph 2, or the vector of paragraph 3.

5. A vaccine comprising the live attenuated recombinant Zika virus, recombinant Zika virus, recombinant Zika virus particle or recombinant Zika virus nucleic acid of paragraph 1, the recombinant, isolated or substantially purified nucleic acid of paragraph 2, the vector of paragraph 3, or the cell or isolate of paragraph 4.

6. A pharmaceutical preparation comprising the live attenuated recombinant Zika virus, recombinant Zika virus, recombinant Zika virus particle or recombinant Zika virus nucleic acid of paragraph 1, the recombinant, isolated or substantially purified nucleic acid of paragraph 2, the vector of paragraph 3, or the cell or isolate of paragraph 4.

7. An immunogenic composition comprising the live attenuated recombinant Zika virus, recombinant Zika virus, recombinant Zika virus particle or recombinant Zika virus nucleic acid of paragraph 1, the recombinant, isolated or substantially purified nucleic acid of paragraph 2, the vector of paragraph 3, or the cell or isolate of paragraph 4.

8. A method of (1) treating a subject having a natural Zika viral infection, (2) reducing the severity of a natural Zika viral infection in a subject, or (3) preventing a subject from contracting a Zika viral infection naturally, said method comprising the step of administering to the subject:

the live attenuated recombinant Zika virus, recombinant Zika virus, recombinant Zika virus particle or recombinant Zika virus nucleic acid of paragraph 1; the recombinant, isolated or substantially purified nucleic acid of paragraph 2; the vector of paragraph 3; the cell or isolate of paragraph 4; the vaccine of paragraph 5; the pharmaceutical preparation of paragraph 6; or the immunogenic composition of paragraph 7.

9. Use of: the live attenuated recombinant Zika virus, recombinant Zika virus, recombinant Zika virus particle or recombinant Zika virus nucleic acid of paragraph 1; the recombinant, isolated or substantially purified nucleic acid of paragraph 2; the vector of paragraph 3; the cell or isolate of paragraph 4; the vaccine of paragraph 5; the pharmaceutical preparation of paragraph 6; or the immunogenic composition of paragraph 7, in the preparation of a medicament for (1) treating a subject having a natural Zika viral infection, (2) reducing the severity of a natural Zika viral infection in a subject, or (3) preventing a subject from contracting a Zika viral infection naturally.

10. A method of generating a live attenuated Zika virus vaccine, recombinant Zika virus, recombinant Zika virus particle or recombinant Zika virus nucleic acid, or recombinant, isolated or substantially purified nucleic acid comprising a partly codon deoptimized Zika viral genome or partly codon deoptimized region thereof, comprising the step of partly codon deoptimizing a Zika viral genome.

11. A method of preparing a vaccine comprising live attenuated recombinant Zika virus, said method comprising the steps of: (1) codon deoptimizing a Zika viral genome to produce a partly codon deoptimized live attenuated Zika virus; and (2) enabling the partly codon deoptimized live attenuated Zika virus to replicate.

12. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized Zika viral genome comprises at least about 200 codon changes compared with wild-type or virulent Zika virus.

13. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized Zika viral genome comprises no more than about 800 codon changes, compared with wild-type or virulent Zika virus.

14. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized Zika viral genome comprises between about 200 and about 800 codon changes, compared with wild-type or virulent Zika virus.

15. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized Zika viral genome comprises a minimum of about 286 codon changes, compared with wild-type or virulent Zika virus.

16. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized Zika viral genome comprises a maximum of about 651 codon changes, compared with wild-type or virulent Zika virus.

17. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized Zika viral genome comprises between about 286 and 651 codon changes in the viral genome, compared with wild-type or virulent Zika virus.

18. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein some or all codon changes of the codon deoptimized Zika viral genome compared with wild-type or virulent Zika virus are situated immediately next to one another, in sequence.

19. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein some or all codon changes of the codon deoptimized Zika viral genome compared with wild-type or virulent Zika virus are spaced apart from each other such that they are not situated immediately next to one another, in sequence.

20. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein some codon changes of the codon deoptimized Zika viral genome compared with wild-type or virulent Zika virus are spaced apart from each other and some of the codon changes are situated immediately next to one another.

21. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein codon deoptimization occurs in no less than about a 1700 nucleotide region of the codon deoptimized Zika viral genome compared with wild-type or virulent Zika virus, and optionally the 1700 nucleotide region is continuous/contiguous or the 1700 nucleotide region is not continuous/not contiguous.

22. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein codon deoptimization occurs in no more than in about a 7900 nucleotide region of the codon deoptimized Zika viral genome compared with wild-type or virulent Zika virus, and optionally the 7900 nucleotide region is continuous/contiguous or the 7900 nucleotide region is not continuous/not contiguous.

23. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein codon deoptimization occurs in a continuous region of the codon deoptimized Zika viral genome compared with wild-type or virulent Zika virus with a length of about 1800 to about 3600 nucleotides.

24. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein codon deoptimization results in no less than about an 1800 nucleotide region of the genome compared with wild-type or virulent Zika virus, with no less than about 250 codon changes within that nucleotide region.

25. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein codon deoptimization results in no more than about a 7900 nucleotide region of the genome compared with wild-type or virulent Zika virus, with no more than about 800 codon changes within that nucleotide region.

26. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein about 20-60% of the coding region of the genome is codon deoptimized compared with wild-type or virulent Zika virus, preferably 18-36% of the genome, compared with wild-type or virulent Zika virus.

27. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the non-structural region of the viral genome is codon deoptimized.

28. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein only the non-structural region of the viral genome is codon deoptimized.

29. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein every 3^{rd} or 4^{th} codon is deoptimized along the nonstructural ZIKV coding region.

30. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein any one or more of the genes NS1, NS2A, NS2B, NS3, NS4A, NS4B and NS5 are codon deoptimized.

31. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein any contiguous genome region from the NS1 to NS5 region corresponding to at least 600 amino acid residues of viral polyprotein is codon deoptimized.

32. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the genes NS1, NS2A, NS2B, NS3, NS4A, NS4B and NS5 are codon deoptimized.

33. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the genes NS1, NS2A, NS2B and NS3 are codon deoptimized.

34. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the gene NS3 is codon deoptimized.

35. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein approximately 700 codon substitutions are made along the entire nonstructural ZIKV coding region compared with wild-type or virulent Zika virus.

36. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimization results in slower polyprotein translation leading to slower replication and, as a result, in attenuation of the virus, compared with wild-type or virulent Zika virus.

37. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein a codon for an amino acid with high codon degeneracy is changed to a synonymous codon that is used least frequently or rarely in the genome of Homo sapiens.

38. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the genome region most rich in codons that can be substituted for rare codon variants is codon deoptimized.

39. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the viral genome prior to codon deoptimization has a very similar nucleotide sequence to a Zika strain associated with microcephaly, preferably Brazilian Zika virus (ZIKV) strain BeH819016.

40. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized genome has the deoptimized codons of the NS3 region of vaccine candidate ZIKV-DO-NS3 as represented by SEQ ID NO:3, 4, 5 or 10.

41. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized genome has about 200 or more of the codon changes of the NS3 region of vaccine candidate ZIKV-DO-NS3 as represented by SEQ ID NO:3, 4, 5 or 10.

42. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized genome has the deoptimized codons of the NS1, NS2A, NS2B, NS3, NS4A, NS4B and/or NS5 regions of the vaccine candidate ZIKV-DO-scattered as represented by SEQ ID NO:6, 7 or 11.

43. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized genome has about 200 or more of the codon changes of the NS1, NS2A, NS2B, NS3, NS4A, NS4B and/or NS5 regions of the vaccine candidate ZIKV-DO-scattered as represented by SEQ ID NO: 6, 7 or 11.

44. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized genome has the deoptimized codons of the NS1, NS2A, NS2B and/or NS3 regions of the vaccine candidate ZIKV-DO as represented by SEQ ID NO:8, 9 or 12.

45. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized genome has about 200 or more of the codon changes of the NS1, NS2A, NS2B and/or NS3 regions of the vaccine candidate ZIKV-DO as represented by SEQ ID NO:8, 9 or 12.

46. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized genome has the deoptimized codons of the nonstructural region as represented by SEQ ID NO:1 or as shown in FIG. 1b.

47. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized genome has 1 or more of the codon changes of SEQ ID NO:1.

48. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized genome has at least 90 percent of the deoptimized codons of the NS3 region of vaccine candidate ZIKV-DO-NS3 as represented by SEQ ID NO:3, 4, 5 or 10. (For clarity, at least 90 percent includes 90, 91, 92, 93, 94, 95, 96, 97, 98, 99 and 100 percent.)

49. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized genome has at least 90 percent of the deoptimized codons of the NS1, NS2A, NS2B, NS3, NS4A, NS4B and NS5 regions of the vaccine candidate ZIKV-DO-scattered as represented by SEQ ID NO:6, 7 or 11.

50. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the codon deoptimized genome has at least 90 percent of the deoptimized codons of the NS1, NS2A, NS2B and NS3 regions of the vaccine candidate ZIKV-DO as represented by SEQ ID NO:8, 9 or 12.

51. The live attenuated recombinant Zika virus, recombinant Zika virus, recombinant Zika virus particle or recombinant Zika virus nucleic acid, the recombinant, isolated or substantially purified nucleic acid, the vector, the cell or isolate, the vaccine, the pharmaceutical preparation, the immunogenic composition, the method, the use, or the method of any one or more of the preceding paragraphs (context permitting), wherein the vaccine, pharmaceutical preparation or immunogenic composition comprises a delivery system, carrier or aid.

Any of the features described herein can be combined in any combination with any one or more of the other features described herein within the scope of the invention.

### EXAMPLES

Construction of ZIKV Vaccine Candidates Using Codon Deoptimization Technology

In order to generate a live attenuated Zika vaccine, we first constructed infectious clones of a Brazilian Zika virus (ZIKV) strain—BeH819016—which has a very similar sequence to those strains associated with microcephaly. Using the infectious clone and codon deoptimization (CD) technology, we inserted a number of changes in the non-structural (NS1, NS2A, NS2B, NS3) regions of the virus, with the objective of decreasing replication efficiency in mammalian cells and rendering the virus attenuated compared to wild-type ZIKV. Using this strategy, we generated a panel of clones for further testing. Clones that could be successfully ‘rescued’ were tested for their ability to replicate in mammalian and mosquito cells. The resulting viruses were strongly attenuated but still produced viral proteins to a level comparable to wild-type virus. Thus, using CD technology, we were able to generate a panel of live attenuated ZIKV vaccine candidates.

Synthetic sequence of BeH819016 strain from Brazil (very close to the strains shown to be associated with microcephaly) was used as the initial ZIKV genome. All the changes were made only in the nonstructural part of ZIKV genome to prevent possible adverse effect on structure of viral antigens which may result from altered dynamics of their translation. Three attenuated candidates were constructed:

1. ZIKV-DO with a codon deoptimized NS1-NS2A-NS2B-NS3 region (see FIG. 1a and SEQ ID:8 and 9). Approximately 3900 bases (36% of the genome) were de-optimized for human cells.

2. ZIKV-DO-NS3 with a codon deoptimized NS3 region (see FIG. 1a and SEQ ID NO: 3, 4 and 5).

3. ZIKV-DO-scattered where deoptimized codons are scattered over all the nonstructural genome part from the beginning of NS1 till the end of NS5 (see FIG. 1a and SEQ ID NO:6 and 7).

### Methods

Process of Deoptimization

In contrast to an optimization process, which can be done using free software or online, there is no publicly available program for CD. Therefore, it was done manually. In case of ZIKV-DO and ZIKV-DO-NS3 every codon in the indicated sequence was analyzed in terms of its usage frequency in Homo sapiens. If the codon was frequent it was manually changed to a synonymous but the less used one. For instance, amino acid Leucine (Leu) can be encoded by six different codons with the following frequencies: UUA—15%, UUG—12%, CUU—12%, CUC—10%, CUA—5%, and CUG—46%. If the Leu codon in the original sequence was represented by highly abundant CUG (46%), it was changed to rare CUA (5%). Some codons were left unchanged: Methionine (Met) and Tryptophan (Trp) as both of them are encoded by only one codon; and, Asparagine (Asn) and Aspartic acid (Asn) as their codons are used at almost the same frequency. Altogether, ˜700 changes were made in the ZIKV-DO genome and ˜350 changes in ZIKV-DO-NS3 genome. In the case of ZIKV-DO-scattered, approximately every 3^{rd} or 4^{th} codon was deoptimized along the entire nonstructural ZIKV coding region. Again, Met, Trp, Asp, and Asn codons were left unchanged. Approximately 700 substitutions were inserted in the case of ZIKV-DO-scattered.

Rescue of Deoptimized ZIKVs

Deoptimized sequences were purchased as synthetic DNA fragments and were used to replace wildtype (wt) counterparts in the initial pCCI-ZIKV-wt clone using appropriate unique restriction sites. Obtained cDNA clones were verified by restriction analysis and sequencing of deoptimized regions. Plasmid DNAs were amplified using E. coli NEB Turbo strain and purified using Macherey-Nagel Xtra Midi preparation kit. Plasmids were linearized using AgeI (BshTI) restriction enzyme and spin-column purified. Capped transcripts, corresponding to viral genome RNAs, were synthesized in vitro with Ambion mMessage-mMachine kit using linearized plasmid DNAs as templates. The quality and integrity of synthesized RNAs was verified by gel-electrophoresis. Obtained in vitro transcription mixtures were used for transfection of Vero E6 cells (derived from African green monkey kidney) by lipofection using Lipofectamine 2000 (Invitrogen) reagent and manufacturer's protocol. Transfected cells were incubated for 14 days and the cells' supernatant was then used for infection of new Vero E6 or Ae. albopictus cells C6/36.

Titration of Codon Deoptimized ZIKVs

All codon deoptimized ZIKV vaccine candidates failed to form plaques in Vero E6 cells indicating that they were attenuated. Their titration was therefore performed using A549Npro cells deficient in intracellular immune response. Ordinary plaque forming assay was used for titration with incubation time of 7 days for plaque formation. Infected cells were stained with crystal violet solution and formed plaques counted to obtain viral titers.

Improved Propagation of Attenuated ZIKV-DO-NS3 Strain with Deoptimized NS3 Region

We used the Vero E6 clone for propagation of the virus. TPCK-treated (N-tosyl-L-phenylalanine chloromethyl ketone) trypsin (at 0.5 μg/ml concentration) increased the titer of ZIKV-DO-NS3. The FBS (fetal bovine serum) content in virus growth media could be reduced to 1% or replaced with 0.2% BSA (bovine serum albumin). ZIKV-DO-NS3 was titrated only on A549NPro cells. The best MOIs (multiplicities of infection) for infection were 0.01-0.1 pfu/cell.

Virus was propagated on Vero E6 cells. 100 mm plates, 37° C. 5% CO₂. Cells were ˜50-80% confluent at the moment of infection. Low MOI was used (0.01-0.1 pfu/cell). Cells were washed with PBS (phosphate buffered saline) and infected in 2 ml of serum-free DMEM (Dulbecco's modified Eagle's medium) for 2 hours with rocking of the plate every 10-15 minutes; then 8 ml virus growth medium (VGA, DMEM+0.2% BSA+Pen-Strep+0.5 μg/ml TPCK) was added (inoculum was not removed). During incubation, plates were gently rocked back and forth 4-5 times every day for the first 5 days to facilitate spread of virus over the plate. Growth media were sampled (approximately 0.5 ml) at Days 7, 10, and 14. Virus titers were determined on A549NPro cells using immuno-plaque assay with anti-ZIKV NS3 rabbit antibody (in house) and IRDye 800CW goat anti-rabbit secondary antibody (LI-COR). Cells were incubated for 96 hours before fixation. Virus titers in samples: Day 7—2×10*7 pfu/ml; Day 10—1.5×10*7 pfu/ml; Day 14-5×10*7 pfu/ml. The samples were also titrated by classical plaque titration on A549NPro cells (incubation time—8 days) with the same or similar results.

Results

Codon Deoptimized ZIKV Genomes

Cloning of codon deoptimized ZIKV genomes resulted in three plasmids, ZIKV-DO, ZIKV-DO-NS3 and ZIKV-DO-scattered, whose coding regions are schematically depicted in FIG. 1a.

A representative computational codon deoptimization is depicted in FIG. 1b.

Rescue of Attenuated ZIKVs with Deoptimized Nonstructural Regions

The deoptimized ZIKV vaccine candidates were rescued in Vero E6 cells and passaged 3 times with no significant cytopathic effect for up to 14 days. No protein expression was detected by western blot in Vero E6 cells (except ZIKV-DO-scattered). Subsequently, ZIKV-DO and ZIKV-DO-NS3 were passaged in mosquito Ae. albopictus cells for 7 days. Protein expression (NS3 and Envelope proteins) for ZIKV-DO and ZIKV-DO-NS3 viruses was confirmed in insect cells by western blot analysis. Supernatants collected from both Vero E6 and C6/36 cells were plaque-titrated on A549NPro cells.

Testing of Vaccines In Vivo

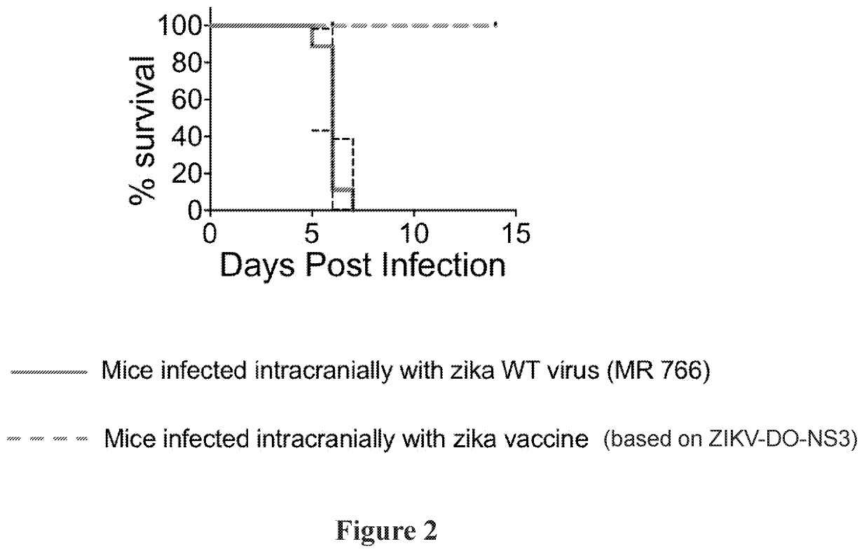

To test the vaccines in vivo, we required a suitable immunocompetent mouse model. Most mouse models of ZIKV infection are based on mice with an impaired immune system, making them inappropriate for vaccine studies. We have been successful in generating an immunocompetent C57BL/6 adult mouse model of ZIKV infection, using ZIKV strain MR766. The model is based on intracranial (i.c) infection of adult wild-type mice with 4×10⁵ PFU ZIKV. Mice show high susceptibility to infection in our model (FIG. 2), with all mice dying by d6 p.i. This high susceptibility to infection makes our mouse model particularly suitable for use in vaccine testing. One vaccine candidate, ZIKA-DO-NS3, was initially selected for further testing. In contrast to the 100% mortality seen in mice infected with ZIKV MR766, mice given 4×10⁵ PFU of the live attenuated ZIKV vaccine based on ZIKA-DO-NS3 showed no mortality. Thus, the vaccine candidate ZIKA-DO-NS3 is extremely safe.

For the experiments with ZIKV-DO-NS3, there were 5 mice per group and each experiment was repeated 3 times.

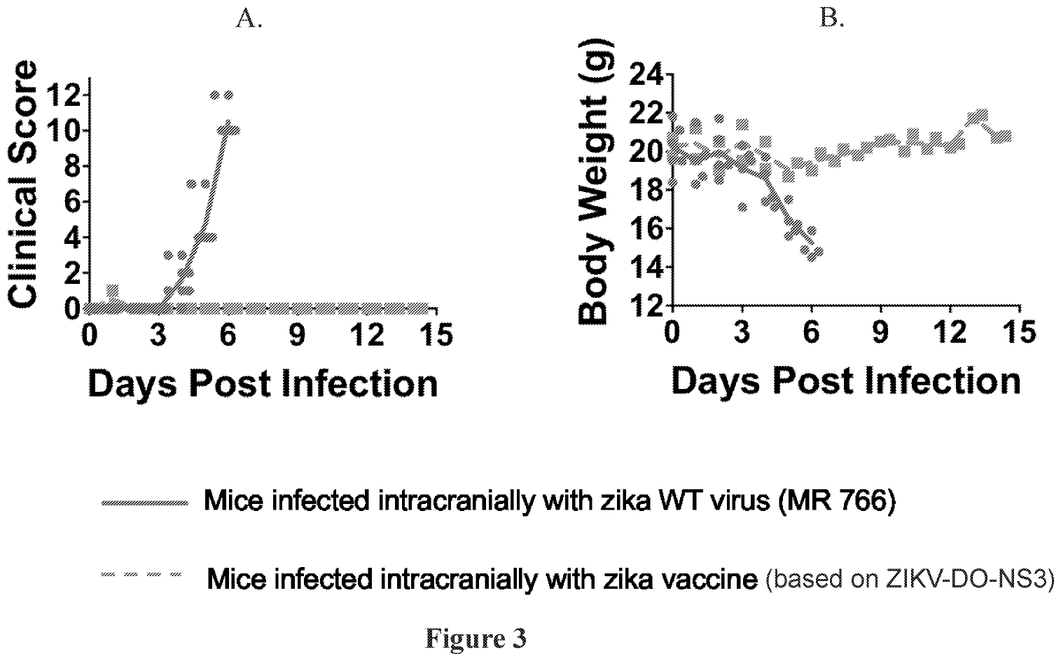

In our mouse model of i.c. ZIKV infection, as seen in FIG. 3, mice show prominent signs of disease, which are measured by clinical score and loss of body weight. Clinical score is measured by assessing and scoring a number of clinical signs: every 5% weight loss scores one point; noticeable hesitation in activity scores one point, significant reduction in activity scores 2 points, move only when pushed scores 3 points (select just one of these three movement assessments); rough fur scores 1 point; hunching scores one point; trembling scores one point; standing on hind limbs scores one point. These scores are added together to give a total clinical score. Disease was assessed in mice infected with the vaccine candidate based on ZIKA-DO-NS3 used in FIG. 2. Mice infected with 4×10⁵ PFU ZIKV MR766 showed a dramatic increase in clinical score and weight loss. In contrast, infection of C57BL/6 mice with 4×10⁵ PFU of the live attenuated ZIKV based on ZIKA-DO-NS3 did not affect clinical score and there was no weight loss. Thus, the vaccine candidate based on ZIKA-DO-NS3 is extremely safe.

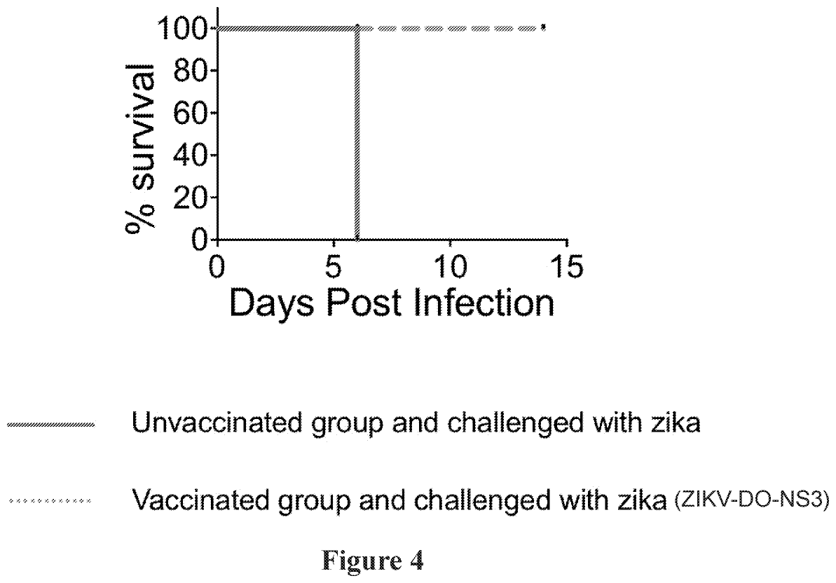

To test vaccine efficacy, as seen in FIG. 4, mice were immunised with 2×10⁴ PFU of the live attenuated vaccine based on ZIKV-DO-NS3 subcutaneously (s.c). Control mice received PBS. We adopted a booster regimen, with s.c immunisation at days 0, 7 and 14. Fourteen days following vaccination, mice were given a lethal i.c challenge with 4×10⁵ PFU ZIKV. The control (non-vaccinated) mice all died within 6 days of infection. In contrast, there was no mortality in the vaccinated mice. n=5 mice per group.

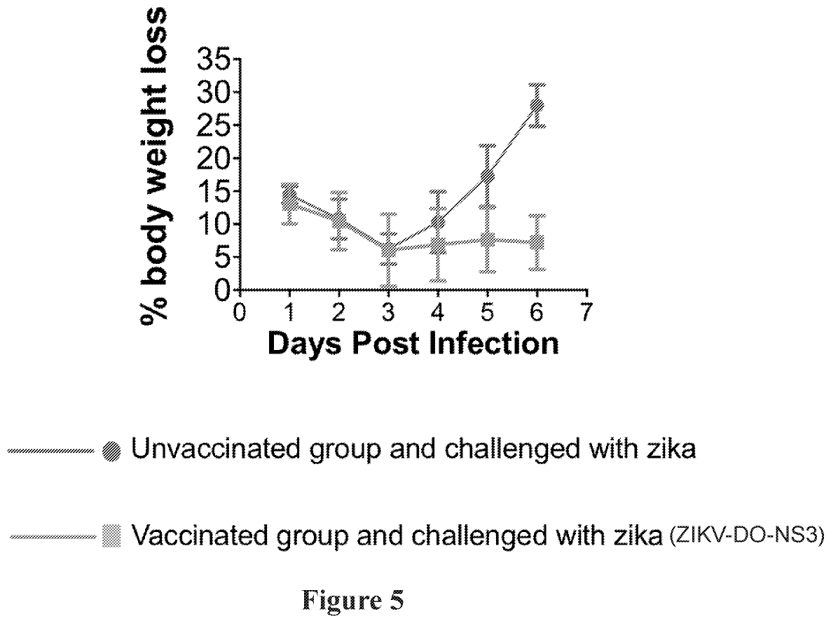

To test vaccine efficacy, as seen in FIG. 5, mice were immunised with the live attenuated vaccine ZIKV-DO-NS3 subcutaneously (s.c). Control mice received PBS. We adopted a booster regimen, with s.c immunisation at days 0, 7 and 14. Fourteen days following vaccination, mice were given a lethal i.c challenge with 4×10⁵ PFU ZIKV. The control (non-vaccinated) mice showed substantial loss of body weight from day 3 until death on day 6. In contrast, there was no loss of body weight in the vaccinated mice. n=5 mice per group.

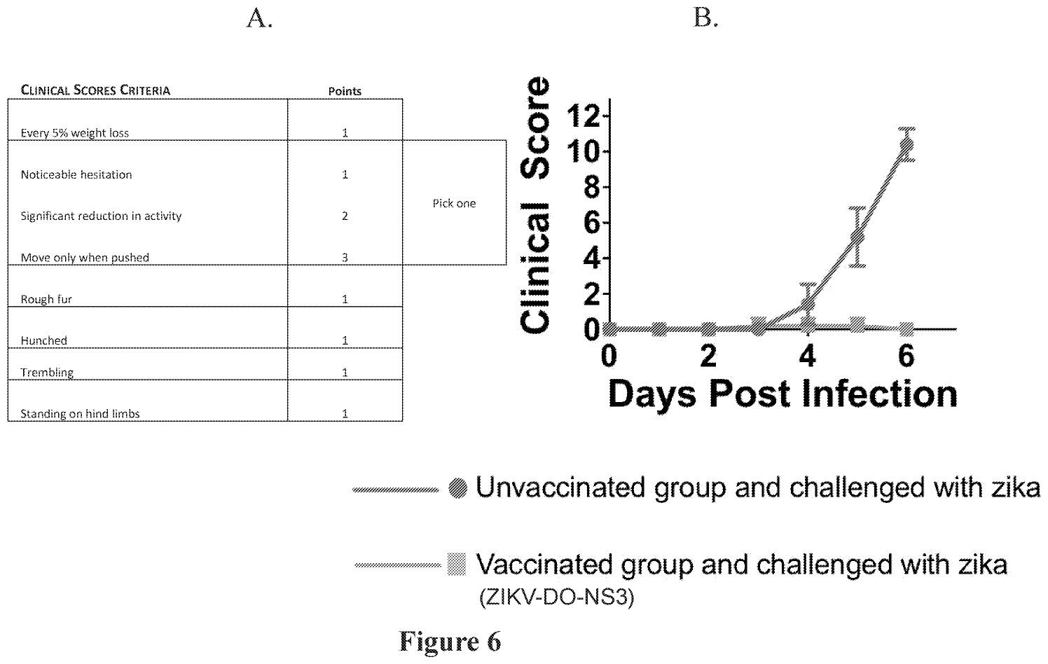

To test vaccine efficacy, as seen in FIG. 6, mice were immunised with the live attenuated vaccine ZIKV-DO-NS3 subcutaneously (s.c). Control mice received PBS. We adopted a booster regimen, with s.c immunisation at days 0, 7 and 14. Fourteen days following vaccination, mice were given a lethal i.c challenge with 4×10⁵ PFU ZIKV. The control (non-vaccinated) mice showed a dramatic increase in clinical score from day 4 until death on day 6. In contrast, there was no increase in clinical score at any time point in the vaccinated mice. n=5 mice per group.

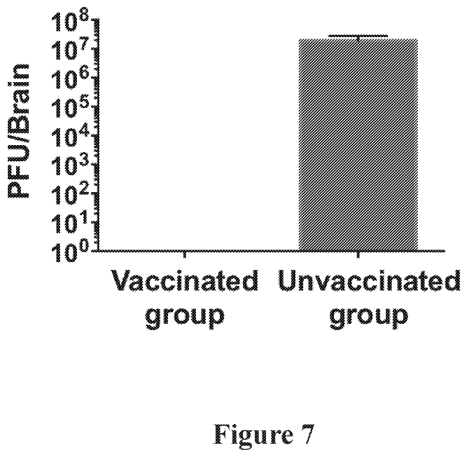

To test vaccine efficacy, as seen in FIG. 7, mice were immunised with the live attenuated vaccine ZIKV-DO-NS3 subcutaneously (s.c). Control mice received PBS. We adopted a booster regimen, with s.c immunisation at days 0, 7 and 14. Fourteen days following vaccination, mice were given a lethal i.c challenge with 4×10⁵ PFU ZIKV. The control (non-vaccinated mice) showed very high levels of ZIKV virus in brain tissue at 6 days p.i. In contrast, there was no detectable virus in the brains of vaccinated mice at day 6. n=5 mice per group.

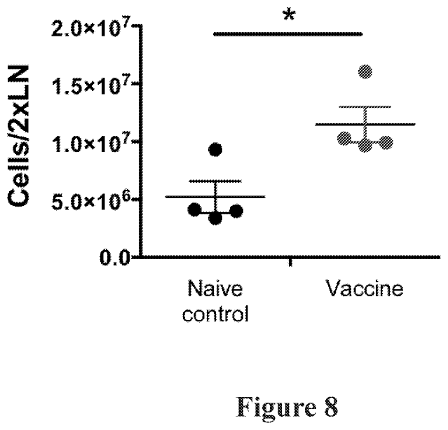

We conducted an initial assessment of immunological mechanisms of protection mediated by the vaccine, and the results are shown in FIG. 8. C57BL/6 mice were immunized s.c with 2×10⁴ PFU of the live attenuated vaccine ZIKV-DO-NS3 on days 0, 7 and 14. The mice were killed one week later and the draining lymph nodes were collected for analysis. There was a significant increase in the cellularity of draining lymph nodes in vaccinated mice compared to non-vaccinated mice (n=5 mice per group).

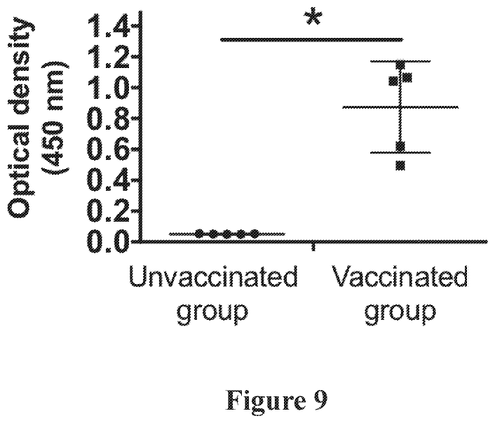

We conducted an initial assessment of immunological mechanisms of protection mediated by the vaccine, and the results are shown in FIG. 9. C57BL/6 mice were immunized s.c with 2×10⁴ PFU of the live attenuated vaccine ZIKV-DO-NS3 on days 0, 7 and 14. The mice were bled and the ZIKV-specific antibody response assessed. Vaccinated mice mounted a strong antibody response against ZIKV. No ZIKV-specific antibodies were detected in non-vaccinated mice (n=5 mice per group).

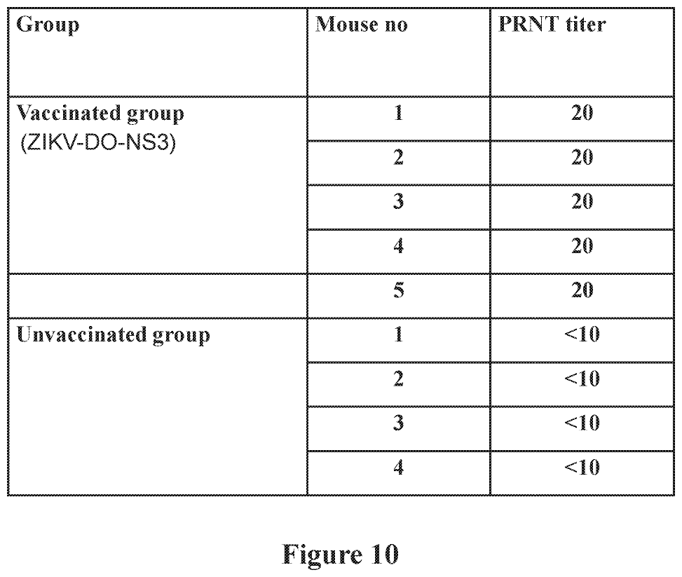

We conducted an initial assessment of immunological mechanisms of protection mediated by the vaccine, and the results are shown in FIG. 10. C57BL/6 mice were immunized s.c with 2×10⁴ PFU of the live attenuated vaccine ZIKV-DO-NS3 on days 0, 7 and 14. The mice were bled and the ZIKV neutralising antibody response assessed. ZIKV-specific neutralising antibodies were induced by the vaccine, as measured using a plaque reduction neutralisation test (PRNT). No ZIKV-specific neutralising antibodies were detected in non-vaccinated mice (n=5 mice per group).

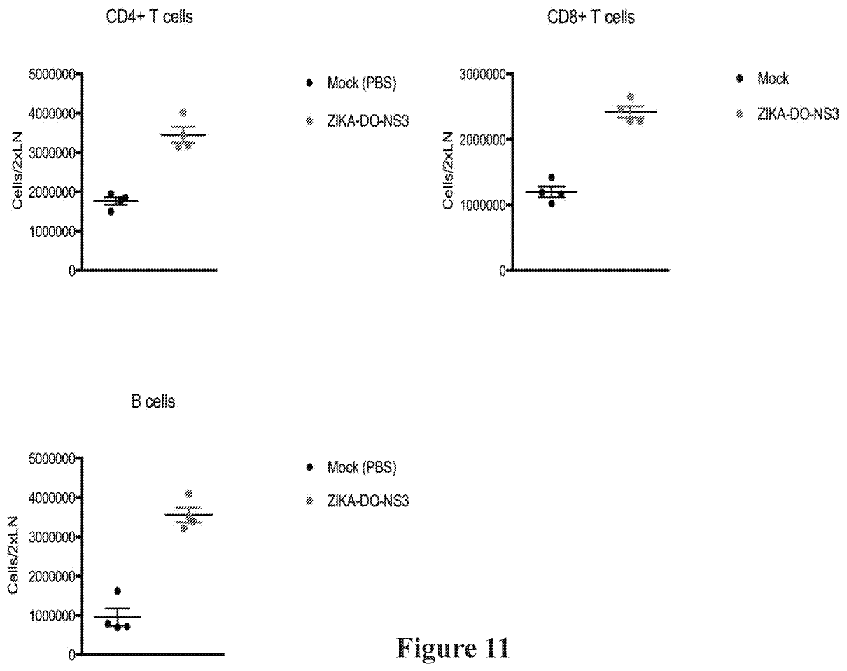

We conducted an assessment of immunological mechanisms of protection mediated by the vaccine, and the results are shown in FIG. 11. We found that s.c inoculation of ZIKV-DO-NS3 induces an immune response in the draining lymph nodes. C57BL/6 mice were immunized s.c with 2×10⁴ PFU of the live attenuated vaccine ZIKV-DO-NS3 on days 0, 7 and 14. Control mice were given PBS. Vaccinated and control mice were euthanized 6 days after the last immunisation. Draining lymph nodes posterior axillary, bilateral regions were collected. Numbers of CD4+ T cells, CD8+ T cells and B cells were quantitated using flow cytometry. Mice immunized with ZIKV-DO-NS3 mounted a strong T and B cell responses. Weak T and B cell responses were detected in non-vaccinated mice (n=4 mice per group).

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention.

In the present specification and claims (if any), the word ‘comprising’ and its derivatives including ‘comprises’ and ‘comprise’ include each of the stated integers but does not exclude the inclusion of one or more further integers.

The reference to any prior art in this specification is not, and should not be taken as an acknowledgement or any form of suggestion that the prior art forms part of the common general knowledge.

1. A composition of matter selected from the group consisting of:
(a) a live attenuated recombinant Zika virus comprising a partly codon deoptimized Zika viral genome;
(b) a recombinant Zika virus comprising a partly codon deoptimized Zika viral genome;
(c) a recombinant Zika virus particle comprising a partly codon deoptimized Zika viral genome;
(d) a recombinant Zika virus nucleic acid comprising a partly codon deoptimized Zika viral genome;
(e) a vaccine containing any one of (a) to (d);
(f) a cell containing any one of (a) to (d);
(g) an isolate containing any one of (a) to (d);
(h) a pharmaceutical preparation containing any one of (a) to (d); and
(i) an immunogenic composition containing any one of (a) to (d).

2-7. (canceled)

8. A method selected from the group consisting of: (1) treating a subject having a natural Zika viral infection; (2) reducing the severity of a natural Zika viral infection in a subject; and (3) preventing a subject from contracting a Zika viral infection naturally, said method comprising the step of administering to the subject the composition of matter of claim 1.

9. (canceled)

10. A method of generating a live attenuated Zika virus vaccine, recombinant Zika virus, recombinant Zika virus particle or recombinant Zika virus nucleic acid, or recombinant, isolated or substantially purified nucleic acid comprising a partly codon deoptimized Zika viral genome or partly codon deoptimized region thereof, comprising the step of partly codon deoptimizing a Zika viral genome.

11. (canceled)

12. The composition of matter of claim 1, wherein the codon deoptimized Zika viral genome comprises codon changes selected from the group consisting of: at least about 200 codon changes compared with wild-type or virulent Zika virus; no more than about 800 codon changes, compared with wild-type or virulent Zika virus; between about 200 and about 800 codon changes, compared with wild-type or virulent Zika virus; a minimum of about 286 codon changes, compared with wild-type or virulent Zika virus; a maximum of about 651 codon changes, compared with wild-type or virulent Zika virus; between about 286 and 651 codon changes in the viral genome, compared with wild-type or virulent Zika virus; codon deoptimization occurs in no less than about a 1700 nucleotide region of the codon deoptimized Zika viral genome compared with wild-type or virulent Zika virus; codon deoptimization occurs in no more than in about a 7900 nucleotide region of the codon deoptimized Zika viral genome compared with wild-type or virulent Zika virus; codon deoptimization occurs in a continuous region of the codon deoptimized Zika viral genome compared with wild-type or virulent Zika virus with a length of about 1800 to about 3600 nucleotides; codon deoptimization results in no less than about an 1800 nucleotide region of the genome compared with wild-type or virulent Zika virus, with no less than about 250 codon changes within that nucleotide region; codon deoptimization results in no more than about a 7900 nucleotide region of the genome compared with wild-type or virulent Zika virus, with no more than about 800 codon changes within that nucleotide region; and, about 20-60% of the coding region of the genome is codon deoptimized compared with wild-type or virulent Zika virus.

13-26. (canceled)

27. The composition of matter of claim 1, wherein the codon deoptimized Zika viral genome comprises codon changes selected from the group consisting of: the non-structural region of the viral genome is codon deoptimized; only the non-structural region of the viral genome is codon deoptimized; every 3rd or 4th codon is deoptimized along the nonstructural ZIKV coding region; any one or more of the genes NS1, NS2A, NS2B, NS3, NS4A, NS4B and NS5 are codon deoptimized; any contiguous genome region from the NS1 to NS5 region corresponding to at least 600 amino acid residues of viral polyprotein is codon deoptimized; the genes NS1, NS2A, NS2B, NS3, NS4A, NS4B and NS5 are codon deoptimized; the genes NS1, NS2A, NS2B and NS3 are codon deoptimized; the gene NS3 is codon deoptimized; approximately 700 codon substitutions are made along the entire nonstructural ZIKV coding region compared with wild-type or virulent Zika virus.

28-39. (canceled)

40. The composition of matter of claim 1, wherein the codon deoptimized Zika viral genome comprises codon changes selected from the group consisting of: the codon deoptimized genome has the deoptimized codons of the NS3 region of vaccine candidate ZIKV-DO-NS3 represented by SEQ ID NO:3, 4, 5 or 10; the codon deoptimized genome has about 200 or more of the codon changes of the NS3 region of vaccine candidate ZIKV-DO-NS3 represented by SEQ ID NO:3, 4, 5 or 10; the codon deoptimized genome has the deoptimized codons of the NS1, NS2A, NS2B, NS3, NS4A, NS4B and/or NS5 regions of the vaccine candidate ZIKV-DO-scattered represented by SEQ ID NO:6, 7 or 11; the codon deoptimized genome has about 200 or more of the codon changes of the NS1, NS2A, NS2B, NS3, NS4A, NS4B and/or NS5 regions of the vaccine candidate ZIKV-DO-scattered represented by SEQ ID NO:6, 7 or 11; the codon deoptimized genome has the deoptimized codons of the NS1, NS2A, NS2B and/or NS3 regions of the vaccine candidate ZIKV-DO represented by SEQ ID NO:8, 9 or 12; the codon deoptimized genome has about 200 or more of the codon changes of the NS1, NS2A, NS2B and/or NS3 regions of the vaccine candidate ZIKV-DO represented by SEQ ID NO:8, 9 or 12; the codon deoptimized genome has the deoptimized codons of the nonstructural region represented by SEQ ID NO:1; the codon deoptimized genome has at least 90 percent of the deoptimized codons of the NS3 region of vaccine candidate ZIKV-DO-NS3 represented by SEQ ID NO:3, 4, 5 or 10; the codon deoptimized genome has at least 90 percent of the deoptimized codons of the NS1, NS2A, NS2B, NS3, NS4A, NS4B and NS5 regions of the vaccine candidate ZIKV-DO-scattered represented by SEQ ID NO:6 or 7; and, the codon deoptimized genome has at least 90 percent of the deoptimized codons of the NS1, NS2A, NS2B and NS3 regions of the vaccine candidate ZIKV-DO represented by SEQ ID NO:8 or 9.

41-51. (canceled)

52. The method of claim 8, wherein the codon deoptimized Zika viral genome comprises codon changes selected from the group consisting of: at least about 200 codon changes compared with wild-type or virulent Zika virus; no more than about 800 codon changes, compared with wild-type or virulent Zika virus; between about 200 and about 800 codon changes, compared with wild-type or virulent Zika virus; a minimum of about 286 codon changes, compared with wild-type or virulent Zika virus; a maximum of about 651 codon changes, compared with wild-type or virulent Zika virus; between about 286 and 651 codon changes in the viral genome, compared with wild-type or virulent Zika virus; codon deoptimization occurs in no less than about a 1700 nucleotide region of the codon deoptimized Zika viral genome compared with wild-type or virulent Zika virus; codon deoptimization occurs in no more than in about a 7900 nucleotide region of the codon deoptimized Zika viral genome compared with wild-type or virulent Zika virus; codon deoptimization occurs in a continuous region of the codon deoptimized Zika viral genome compared with wild-type or virulent Zika virus with a length of about 1800 to about 3600 nucleotides; codon deoptimization results in no less than about an 1800 nucleotide region of the genome compared with wild-type or virulent Zika virus, with no less than about 250 codon changes within that nucleotide region; codon deoptimization results in no more than about a 7900 nucleotide region of the genome compared with wild-type or virulent Zika virus, with no more than about 800 codon changes within that nucleotide region; and, about 20-60% of the coding region of the genome is codon deoptimized compared with wild-type or virulent Zika virus.

53. The method of claim 8, wherein the codon deoptimized Zika viral genome comprises codon changes selected from the group consisting of: the non-structural region of the viral genome is codon deoptimized; only the non-structural region of the viral genome is codon deoptimized; every 3rd or 4th codon is deoptimized along the nonstructural ZIKV coding region; any one or more of the genes NS1, NS2A, NS2B, NS3, NS4A, NS4B and NS5 are codon deoptimized; any contiguous genome region from the NS1 to NS5 region corresponding to at least 600 amino acid residues of viral polyprotein is codon deoptimized; the genes NS1, NS2A, NS2B, NS3, NS4A, NS4B and NS5 are codon deoptimized; the genes NS1, NS2A, NS2B and NS3 are codon deoptimized; the gene NS3 is codon deoptimized; approximately 700 codon substitutions are made along the entire nonstructural ZIKV coding region compared with wild-type or virulent Zika virus.

54. The method of claim 8, wherein the codon deoptimized Zika viral genome comprises codon changes selected from the group consisting of: the codon deoptimized genome has the deoptimized codons of the NS3 region of vaccine candidate ZIKV-DO-NS3 represented by SEQ ID NO:3, 4, 5 or 10, the codon deoptimized genome has about 200 or more of the codon changes of the NS3 region of vaccine candidate ZIKV-DO-NS3 represented by SEQ ID NO:3, 4, 5 or 10; the codon deoptimized genome has the deoptimized codons of the NS1, NS2A, NS2B, NS3, NS4A, NS4B and/or NS5 regions of the vaccine candidate ZIKV-DO-scattered represented by SEQ ID NO:6, 7 or 11; the codon deoptimized genome has about 200 or more of the codon changes of the NS1, NS2A, NS2B, NS3, NS4A, NS4B and/or NS5 regions of the vaccine candidate ZIKV-DO-scattered represented by SEQ ID NO:6, 7 or 11; the codon deoptimized genome has the deoptimized codons of the NS1, NS2A, NS2B and/or NS3 regions of the vaccine candidate ZIKV-DO represented by SEQ ID NO:8, 9 or 12; the codon deoptimized genome has about 200 or more of the codon changes of the NS1, NS2A, NS2B and/or NS3 regions of the vaccine candidate ZIKV-DO represented by SEQ ID NO:8, 9 or 12; the codon deoptimized genome has the deoptimized codons of the nonstructural region represented by SEQ ID NO:1; the codon deoptimized genome has at least 90 percent of the deoptimized codons of the NS3 region of vaccine candidate ZIKV-DO-NS3 represented by SEQ ID NO:3, 4, 5 or 10; the codon deoptimized genome has at least 90 percent of the deoptimized codons of the NS1, NS2A, NS2B, NS3, NS4A, NS4B and NS5 regions of the vaccine candidate ZIKV-DO-scattered represented by SEQ ID NO:6 or 7; and, the codon deoptimized genome has at least 90 percent of the deoptimized codons of the NS1, NS2A, NS2B and NS3 regions of the vaccine candidate ZIKV-DO represented by SEQ ID NO:8 or 9.

55. The method of claim 10, wherein the codon deoptimized Zika viral genome comprises codon changes selected from the group consisting of: at least about 200 codon changes compared with wild-type or virulent Zika virus; no more than about 800 codon changes, compared with wild-type or virulent Zika virus; between about 200 and about 800 codon changes, compared with wild-type or virulent Zika virus; a minimum of about 286 codon changes, compared with wild-type or virulent Zika virus; a maximum of about 651 codon changes, compared with wild-type or virulent Zika virus; between about 286 and 651 codon changes in the viral genome, compared with wild-type or virulent Zika virus; codon deoptimization occurs in no less than about a 1700 nucleotide region of the codon deoptimized Zika viral genome compared with wild-type or virulent Zika virus; codon deoptimization occurs in no more than in about a 7900 nucleotide region of the codon deoptimized Zika viral genome compared with wild-type or virulent Zika virus; codon deoptimization occurs in a continuous region of the codon deoptimized Zika viral genome compared with wild-type or virulent Zika virus with a length of about 1800 to about 3600 nucleotides; codon deoptimization results in no less than about an 1800 nucleotide region of the genome compared with wild-type or virulent Zika virus, with no less than about 250 codon changes within that nucleotide region; codon deoptimization results in no more than about a 7900 nucleotide region of the genome compared with wild-type or virulent Zika virus, with no more than about 800 codon changes within that nucleotide region; and, about 20-60% of the coding region of the genome is codon deoptimized compared with wild-type or virulent Zika virus.

56. The method of claim 10, wherein the codon deoptimized Zika viral genome comprises codon changes selected from the group consisting of: the non-structural region of the viral genome is codon deoptimized; only the non-structural region of the viral genome is codon deoptimized; every 3rd or 4th codon is deoptimized along the nonstructural ZIKV coding region; any one or more of the genes NS1, NS2A, NS2B, NS3, NS4A, NS4B and NS5 are codon deoptimized; any contiguous genome region from the NS1 to NS5 region corresponding to at least 600 amino acid residues of viral polyprotein is codon deoptimized; the genes NS1, NS2A, NS2B, NS3, NS4A, NS4B and NS5 are codon deoptimized; the genes NS1, NS2A, NS2B and NS3 are codon deoptimized; the gene NS3 is codon deoptimized; approximately 700 codon substitutions are made along the entire nonstructural ZIKV coding region compared with wild-type or virulent Zika virus.

57. The method of claim 10, wherein the codon deoptimized Zika viral genome comprises codon changes selected from the group consisting of: the codon deoptimized genome has the deoptimized codons of the NS3 region of vaccine candidate ZIKV-DO-NS3 represented by SEQ ID NO:3, 4, 5 or 10; the codon deoptimized genome has about 200 or more of the codon changes of the NS3 region of vaccine candidate ZIKV-DO-NS3 represented by SEQ ID NO:3, 4, 5 or 10; the codon deoptimized genome has the deoptimized codons of the NS1, NS2A, NS2B, NS3, NS4A, NS4B and/or NS5 regions of the vaccine candidate ZIKV-DO-scattered represented by SEQ ID NO:6, 7 or 11; the codon deoptimized genome has about 200 or more of the codon changes of the NS1, NS2A, NS2B, NS3, NS4A, NS4B and/or NS5 regions of the vaccine candidate ZIKV-DO-scattered represented by SEQ ID NO:6, 7 or 11; the codon deoptimized genome has the deoptimized codons of the NS1, NS2A, NS2B and/or NS3 regions of the vaccine candidate ZIKV-DO represented by SEQ ID NO:8, 9 or 12; the codon deoptimized genome has about 200 or more of the codon changes of the NS1, NS2A, NS2B and/or NS3 regions of the vaccine candidate ZIKV-DO represented by SEQ ID NO:8, 9 or 12; the codon deoptimized genome has the deoptimized codons of the nonstructural region represented by SEQ ID NO: 1; the codon deoptimized genome has at least 90 percent of the deoptimized codons of the NS3 region of vaccine candidate ZIKV-DO-NS3 represented by SEQ ID NO:3, 4, 5 or 10; the codon deoptimized genome has at least 90 percent of the deoptimized codons of the NS1, NS2A, NS2B, NS3, NS4A, NS4B and NS5 regions of the vaccine candidate ZIKV-DO-scattered represented by SEQ ID NO:6 or 7; and, the codon deoptimized genome has at least 90 percent of the deoptimized codons of the NS1, NS2A, NS2B and NS3 regions of the vaccine candidate ZIKV-DO represented by SEQ ID NO:8 or 9.

58. The composition of matter of claim 1, being the vaccine containing the live attenuated recombinant Zika virus comprising a partly codon deoptimized Zika viral genome, wherein the codon deoptimized Zika viral genome comprises codon changes selected from the group consisting of: the non-structural region of the viral genome is codon deoptimized; only the non-structural region of the viral genome is codon deoptimized; every 3rd or 4th codon is deoptimized along the nonstructural ZIKV coding region; any one or more of the genes NS1, NS2A, NS2B, NS3, NS4A, NS4B and NS5 are codon deoptimized; any contiguous genome region from the NS1 to NS5 region corresponding to at least 600 amino acid residues of viral polyprotein is codon deoptimized; the genes NS1, NS2A, NS2B, NS3, NS4A, NS4B and NS5 are codon deoptimized; the genes NS1, NS2A, NS2B and NS3 are codon deoptimized; the gene NS3 is codon deoptimized; approximately 700 codon substitutions are made along the entire nonstructural ZIKV coding region compared with wild-type or virulent Zika virus.

59. The composition of matter of claim 1, wherein the codon deoptimized Zika viral genome comprises codon changes selected from the group consisting of: the codon deoptimized genome has the deoptimized codons of the NS3 region of vaccine candidate ZIKV-DO-NS3 represented by SEQ ID NO:3, 4, 5 or 10; the codon deoptimized genome has about 200 or more of the codon changes of the NS3 region of vaccine candidate ZIKV-DO-NS3 represented by SEQ ID NO:3, 4, 5 or 10; the codon deoptimized genome has the deoptimized codons of the NS1, NS2A, NS2B, NS3, NS4A, NS4B and/or NS5 regions of the vaccine candidate ZIKV-DO-scattered represented by SEQ ID NO:6, 7 or 11; the codon deoptimized genome has about 200 or more of the codon changes of the NS1, NS2A, NS2B, NS3, NS4A, NS4B and/or NS5 regions of the vaccine candidate ZIKV-DO-scattered represented by SEQ ID NO:6, 7 or 11; the codon deoptimized genome has the deoptimized codons of the NS1, NS2A, NS2B and/or NS3 regions of the vaccine candidate ZIKV-DO represented by SEQ ID NO:8, 9 or 12; the codon deoptimized genome has about 200 or more of the codon changes of the NS1, NS2A, NS2B and/or NS3 regions of the vaccine candidate ZIKV-DO represented by SEQ ID NO:8, 9 or 12; the codon deoptimized genome has the deoptimized codons of the nonstructural region represented by SEQ ID NO:1; the codon deoptimized genome has at least 90 percent of the deoptimized codons of the NS3 region of vaccine candidate ZIKV-DO-NS3 represented by SEQ ID NO:3, 4, 5 or 10; the codon deoptimized genome has at least 90 percent of the deoptimized codons of the NS1, NS2A, NS2B, NS3, NS4A, NS4B and NS5 regions of the vaccine candidate ZIKV-DO-scattered represented by SEQ ID NO:6 or 7; and, the codon deoptimized genome has at least 90 percent of the deoptimized codons of the NS1, NS2A, NS2B and NS3 regions of the vaccine candidate ZIKV-DO represented by SEQ ID NO:8 or 9.

60. The composition of matter of claim 1, wherein the codon deoptimized Zika viral genome comprises codon changes selected from the group consisting of: the genes NS1, NS2A, NS2B, NS3, NS4A, NS4B and NS5 are codon deoptimized; the genes NS1, NS2A, NS2B and NS3 are codon deoptimized; and, the gene NS3 is codon deoptimized.

61. The composition of matter of claim 60, wherein the codon deoptimized genome has the deoptimized codons of the nonstructural region represented by SEQ ID NO:3.

62. The method of claim 8, wherein said method is for preventing a subject from contracting a Zika viral infection naturally, said method comprising the step of administering to the subject the composition of matter of claim 58.

63. The method of claim 62, said method comprising the step of administering to the subject the composition of matter of claim 59.

64. The method of claim 62, said method comprising the step of administering to the subject the composition of matter of claim 60.

65. The method of claim 62, said method comprising the step of administering to the subject the composition of matter of claim 61.


SEQUENCE LISTING (12 sequences)

SEQ ID NO: 1 - DNA, 1050 bp, Artificial Sequence
  ZIKV-DO-scattered vaccine candidate, computational codon deoptimization of nonstructural ZIKV region NS3
caaagaagtaaaaaaaggggagaccacggatggagtatacagagtaatgacgcgtagactgctaggttcgacacaagttggtgtaggagttatgcaagaaggggtctttcatactatgtggcatgtcacaaaaggttccgcgctgcgtagcggtgaaggtagacttgatccgtactggggagatgtaaagcaggatctagtatcatactgtggtccgtggaagctagatgcggcctgggacggtcacagcgaggtacagctcttggcggtaccccccggagaaagagcgaggaatatccagactctacccggaatatttaaaacaaaggatggtgacattggagcggtagcgctggattatccagcaggaacgtcaggatctccgatcctagacaaatgtgggagagtaataggactttatggtaatggggtcgtaatcaaaaatggtagttatgttagtgcgatcacccaaggtaggagggaagaagaaactcctgttgaatgcttcgagccgtcgatgctgaaaaagaagcagctaacggtcttagacttacatcctggagcggggaaaacccgaagagttcttccggaaatagtccgtgaagcgataaaaacacgtctccgtactgtaatcttagctccgaccagggttgtagctgctgaaatggaagaggcccttcgtgggcttccagtacgttatatgacgacagcagtcaatgtaacccactctggtacagaaatcgttgacttaatgtgtcatgccacctttacttcacgtctactacaaccaatcagagttcccaactataatctatatattatggatgaagcccacttcacggatccctcaagtatagcggcaagaggatatatttcaacaagggttgaaatgggcgaggcggcggccatcttcatgacggccacgccaccgggaacccgtgatgcatttccggattccaactcaccgattatggacacggaagtggaagttccagagagagcgtggagctcaggttttgattgggtaacggatcattcgggaaaaacagt

SEQ ID NO: 2 - DNA, 7890 bp, Artificial Sequence
  Zika virus wt region NS1 to NS5
gatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgatcctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttactcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaatccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaagaagagactcctgttgagtgcttcgagccctcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagatagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccataatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgctgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctagaaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtataacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttagaaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcagatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaa

SEQ ID NO: 3 - DNA, 1851 bp, Artificial Sequence
  Vaccine candidate ZIKV-DO-NS3 nonstructural region nucleotide sequence, showing the codon deoptimized NS3 region
agtggtgcgctctgggatgtccccgcgcccaaggaagtaaaaaagggtgagaccacggatggcgtctaccgagtaatgacccgtcgactactcggttcgacgcaagtaggcgtcggcgtaatgcaagagggtgtattccacaccatgtggcatgtaacgaaaggctcggcgctacgatccggtgaaggtcgattggatccgtactggggcgatgtaaagcaagatctagtctcgtactgtggtccgtggaagctcgatgccgcctgggacggtcactccgaggtccagttattggccgtccccccgggcgagcgagcgcgcaatatacaaactctacccggcatattcaagacgaaggatggtgacattggcgcggtagcgctagattacccggcgggcacttcgggctcgccgatactcgacaagtgtggtcgagtcataggcttgtatggtaatggtgtagtcataaaaaatggtagttatgtaagtgccataacccaaggtcgccgcgaagaagagacccccgtagagtgcttcgagccctcgatgctaaagaagaagcaactcactgtattagacttgcatcccggcgcgggtaaaacccgccgagtattgcccgaaatagtacgtgaagccataaaaacgcgattacgtaccgtcatattagcgccgacccgcgtagtagcggcggaaatggaggaggccttgcgaggtttgccggtccgttatatgacgacggcggtaaatgtaacccattcgggcacggaaatagtagacttaatgtgccatgccaccttcacctcgcgtctcctccagccgatacgagtacccaattataatctatatattatggatgaggcccatttcacggatccctcgagtatagcggcgcgaggctacatttcgacgcgcgtagagatgggtgaggcggcggccatattcatgaccgccacgccgccgggcacccgtgacgcgttcccggactcgaattcgccgattatggacaccgaagtcgaagtaccggagcgagcctggtcctcgggttttgattgggtcacggatcattcgggcaaaacggtatggttcgtaccgtccgtccgcaatggtaatgagatagcggcgtgtctaacgaaggcgggcaaacgggtaatacagttatcccgaaagaccttcgagacggagttccaaaaaacgaaacatcaagagtgggacttcgtagtcacgaccgacatttcggagatgggtgccaatttcaaagcggaccgtgtaatagattcgcgccgatgcctcaagccggtaatattggatggtgagcgagtaattctagcgggccccatgcccgtaacgcatgcctccgcggcccaacgccgcggtcgcataggtcgcaatcccaataaacccggcgatgagtatctatatggcggtggttgcgcggagaccgacgaagaccatgcgcattggttggaagcgcgaatgttattggacaatatttacttacaagatggtttaatagcctcgttatatcgacccgaggccgacaaagtagcggccattgagggcgagttcaagttgcgcacggagcaacgcaagaccttcgtcgaattaatgaaacgaggcgatttgcccgtatggctagcctatcaagtagcgtcggccggcataacctacacggatcgacgatggtgcttcgatggtacgaccaataataccataatggaagatagtgtgccggcagaggtgtggaccagacacggagagaaaagagtgctcaaaccgaggtggatggacgccagagtttgttcagatcatgcggccctgaagtcattcaaggagtttgccgctgggaaaaga

SEQ ID NO: 4 - DNA, 1851 bp, Artificial Sequence
  Vaccine candidate ZIKV-DO-NS3 nonstructural region nucleotide sequence, showing the entire codon deoptimized NS3 region
agtggtgcgctctgggatgtccccgcgcccaaggaagtaaaaaagggtgagaccacggatggcgtctaccgagtaatgacccgtcgactactcggttcgacgcaagtaggcgtcggcgtaatgcaagagggtgtattccacaccatgtggcatgtaacgaaaggctcggcgctacgatccggtgaaggtcgattggatccgtactggggcgatgtaaagcaagatctagtctcgtactgtggtccgtggaagctcgatgccgcctgggacggtcactccgaggtccagttattggccgtcccgccgggcgagcgagcgcgcaatatacaaactctacccggcatattcaagacgaaggatggtgacattggcgcggtagcgctagattacccggcgggcacttcgggctcgccgatactcgacaagtgtggtcgagtcataggcttgtatggtaatggtgtagtcataaaaaatggtagttatgtaagtgccataacccaaggtcgccgcgaagaagagacccccgtagagtgcttcgagccctcgatgctaaagaagaagcaactcactgtattagacttgcatcccggcgcgggtaaaacccgccgagtattgcccgaaatagtacgtgaagccataaaaacgcgattacgtaccgtcatattagcgccgacccgcgtagtagcggcggaaatggaggaggccttgcgaggtttgccagtccgttatatgacgacggcggtaaatgtaacccattcgggcacggaaatagtagacttaatgtgccatgccaccttcacctcgcgtctcctccagccgatacgagtacccaattataatctatatattatggatgaggcccatttcacggatccctcgagtatagcggcgcgaggctacatttcgacgcgcgtagagatgggtgaggcggcggccatattcatgaccgccacgccgccgggcacccgtgacgcgttcccggactcgaattcgccgattatggacaccgaagtcgaagtaccggagcgagcctggtcctcgggttttgattgggtcacggatcattcgggcaaaacggtatggttcgtaccgtccgtccgcaatggtaatgagatagcggcgtgtctaacgaaggcgggcaaacgggtaatacagttatcccgaaagaccttcgagacggagttccaaaaaacgaaacatcaagagtgggacttcgtagtcacgaccgacatttcggagatgggtgccaatttcaaagcagaccgtgtaatagattcgcgccgatgcctcaagccggtaatattggatggtgagcgagtaattctagcgggccccatgcccgtaacgcatgcctccgcggcccaacgccgcggtcgcataggtcgcaatcccaataaacccggcgatgagtatctatatggcggtggttgcgcggagaccgacgaagaccatgcgcattggttggaagcgcgaatgttattggacaatatttacttacaagatggtttaatagcctcgttatatcgacccgaggccgacaaagtagcggccattgagggcgagttcaagttgcgcacggagcaacgcaagaccttcgtcgaattaatgaaacgaggcgatttgcccgtatggctagcctatcaagtagcgtcggcaggtataacctacacggatcgacgatggtgcttcgatggtacgaccaataataccataatggaagatagtgtgccggcagaggtgtggaccagacacggagagaaaagagtgctcaaaccgaggtggatggacgccagagtttgttcagatcatgcggccctgaagtcattcaaggagtttgccgctgggaaaaga

SEQ ID NO: 5 - DNA, 7890 bp, Artificial Sequence
  The entire NS3 region of vaccine candidate ZIKV-DO-NS3 is shown together with all flanking nonstructural regions (NS1 to NS5)
gatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgatcctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttactcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaatccaatagccataccctttgcagctggagcgtggtacgtatacgtgaagactggaaaaaggagtggtgcgctctgggatgtccccgcgcccaaggaagtaaaaaagggtgagaccacggatggcgtctaccgagtaatgacccgtcgactactcggttcgacgcaagtaggcgtcggcgtaatgcaagagggtgtattccacaccatgtggcatgtaacgaaaggctcggcgctacgatccggtgaaggtcgattggatccgtactggggcgatgtaaagcaagatctagtctcgtactgtggtccgtggaagctcgatgccgcctgggacggtcactccgaggtccagttattggccgtcccgccgggcgagcgagcgcgcaatatacaaactctacccggcatattcaagacgaaggatggtgacattggcgcggtagcgctagattacccggcgggcacttcgggctcgccgatactcgacaagtgtggtcgagtcataggcttgtatggtaatggtgtagtcataaaaaatggtagttatgtaagtgccataacccaaggtcgccgcgaagaagagacccccgtagagtgcttcgagccctcgatgctaaagaagaagcaactcactgtattagacttgcatcccggcgcgggtaaaacccgccgagtattgcccgaaatagtacgtgaagccataaaaacgcgattacgtaccgtcatattagcgccgacccgcgtagtagcggcggaaatggaggaggccttgcgaggtttgccagtccgttatatgacgacggcggtaaatgtaacccattcgggcacggaaatagtagacttaatgtgccatgccaccttcacctcgcgtctcctccagccgatacgagtacccaattataatctatatattatggatgaggcccatttcacggatccctcgagtatagcggcgcgaggctacatttcgacgcgcgtagagatgggtgaggcggcggccatattcatgaccgccacgccgccgggcacccgtgacgcgttcccggactcgaattcgccgattatggacaccgaagtcgaagtaccggagcgagcctggtcctcgggttttgattgggtcacggatcattcgggcaaaacggtatggttcgtaccgtccgtccgcaatggtaatgagatagcggcgtgtctaacgaaggcgggcaaacgggtaatacagttatcccgaaagaccttcgagacggagttccaaaaaacgaaacatcaagagtgggacttcgtagtcacgaccgacatttcggagatgggtgccaatttcaaagcagaccgtgtaatagattcgcgccgatgcctcaagccggtaatattggatggtgagcgagtaattctagcgggccccatgcccgtaacgcatgcctccgcggcccaacgccgcggtcgcataggtcgcaatcccaataaacccggcgatgagtatctatatggcggtggttgcgcggagaccgacgaagaccatgcgcattggttggaagcgcgaatgttattggacaatatttacttacaagatggtttaatagcctcgttatatcgacccgaggccgacaaagtagcggccattgagggcgagttcaagttgcgcacggagcaacgcaagaccttcgtcgaattaatgaaacgaggcgatttgcccgtatggctagcctatcaagtagcgtcggcaggtataacctacacggatcgacgatggtgcttcgatggtacgaccaataataccataatggaagatagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccataatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgctgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctagaaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtataacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttagaaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcagatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaa

SEQ ID NO: 6 - DNA, 7890 bp, Artificial Sequence
  Vaccine candidate ZIKV-DO-scattered entire nonstructural region
gatgtagggtgctcggtagacttctcaaagaaggaaacgagatgcggtacgggggtattcgtctataacgatgttgaagcctggcgtgacaggtacaaataccatcctgattccccccgtcgattggcagcagcggtcaagcaagcgtgggaagatggtatatgcgggatctcgtctgtttcacgtatggaaaacataatgtggagatcggtagaaggggagctaaacgcaatcctagaagagaatggtgttcaactgacggtagttgtaggatctgtaaaaaacccgatgtggagaggtccgcagagattgccggtacctgtaaacgagctgccccacggttggaaggcttggggtaaatcgtacttcgtaagagcagcaaaaacaaataactcgtttgtcgtggatggtgatacactgaaggaatgtccactcaaacatcgtgcatggaactcgtttcttgtagaggatcatggtttcggggtatttcatactagtgtctggctaaaggttagagaagattattcgttagagtgtgatccggccgttattggtacagctgttaaaggaaaggaggcggtacacagtgatctaggttactggattgaaagtgagaagaatgatacatggaggctaaagagggcccatctaatcgagatgaaaacgtgtgaatggccgaagtcccacacgttgtggacagatggtatagaagagtcggatctgatcataccgaagtctttagcggggccactcagtcatcacaatacgagagagggctataggacccaaatgaaaggtccatggcactcggaagagcttgaaatacggtttgaggaatgtccaggcactaaagtccacgtggaagaaacatgtggtacaagaggaccgtctctgagatcgaccactgcaagtggaagggtaatcgaggaatggtgttgcagggagtgcacgatgcccccactatcgttccgggcgaaagatggctgttggtatggtatggagatacgtcccaggaaagaaccggaaagcaacttagtacgttcaatggtaactgcaggatcgactgatcacatggatcacttctcccttggagtacttgtaatcctgctcatggtacaggaagggctaaagaagagaatgacgacaaagatcataataagcacatcgatggcagtactggtagctatgatactgggaggattttcgatgagtgacctagctaagcttgcgattttgatgggtgcgaccttcgcggaaatgaatactggaggagatgtagcgcatctggcgctaatagcggcatttaaagtcagaccggcgttgctggtatcgttcatcttccgtgctaattggacgccccgtgaatcgatgctgctggcgttggcctcgtgtctattgcaaactgcgatatccgccttggaaggtgacctgatggtactcatcaatggttttgcgttggcctggttagcaatacgagcgatggtagttccacgcacggataacatcacgttggcaatcctagctgctctgacgccactggcccgtggcacactgcttgtagcgtggagagcgggccttgctacgtgcggggggtttatgctactctctctgaaaggaaaaggcagtgtaaagaagaacttaccgtttgtcatggcgctgggactaacggctgtaaggctggtcgatcccatcaacgtagtaggactgctgttactaacaaggagtgggaaacggagctggccgcctagcgaagtactaacagctgttggtctgatatgcgcattggcgggagggttcgcgaaggcagatatagaaatggctgggccgatggccgcggtaggtctgctaatagtcagttacgtagtctcaggaaaaagtgtggacatgtatattgaaagagcgggtgacatcacatgggaaaaagatgcggaagtaactggaaacagtccgcggctcgatgtagcgctagatgaaagtggtgatttttccctggtagaggatgacggtccccccatgagagaaatcatactcaaagtagtcctgatgacgatctgtggcatgaatccgatagccataccgtttgcagctggtgcgtggtacgtatacgtaaagactggaaaacgtagtggtgctctatgggatgtacctgctcccaaagaagtaaaaaaaggggagaccacggatggagtatacagagtaatgacgcgtagactgctaggttcgacacaagttggtgtaggagttatgcaagaaggggtctttcatactatgtggcatgtcacaaaaggttccgcgctgcgtagcggtgaaggtagacttgatccgtactggggagatgtaaagcaggatctagtatcatactgtggtccgtggaagctagatgcggcctgggacggtcacagcgaggtacagctcttggcggtaccccccggagaaagagcgaggaatatccagactctacccggaatatttaaaacaaaggatggtgacattggagcggtagcgctggattatccagcaggaacgtcaggatctccgatcctagacaaatgtgggagagtaataggactttatggtaatggggtcgtaatcaaaaatggtagttatgttagtgcgatcacccaaggtaggagggaagaagaaactcctgttgaatgcttcgagccgtcgatgctgaaaaagaagcagctaacggtcttagacttacatcctggagcggggaaaacccgaagagttcttccggaaatagtccgtgaagcgataaaaacacgtctccgtactgtaatcttagctccgaccagggttgtagctgctgaaatggaagaggcccttcgtgggcttccagtacgttatatgacgacagcagtcaatgtaacccactctggtacagaaatcgttgacttaatgtgtcatgccacctttacttcacgtctactacaaccaatcagagttcccaactataatctatatattatggatgaagcccacttcacggatccctcaagtatagcggcaagaggatatatttcaacaagggttgaaatgggcgaggcggcggccatcttcatgacggccacgccaccgggaacccgtgatgcatttccggattccaactcaccgattatggacacggaagtggaagttccagagagagcgtggagctcaggttttgattgggtaacggatcattcgggaaaaacagtttggtttgttccgagcgtgaggaatggcaatgagatagcagcttgtctaacaaaggctggtaaacgggtcatacaactcagcagaaaaacttttgaaacagagttccaaaaaacaaaacatcaagaatgggactttgttgttacaactgacatatcagagatgggtgccaactttaaagctgaccgtgtcatagattcgaggagatgcctaaagccggtcatactagatggcgagcgagtcattctggcgggacccatgccggtcacacatgcgagcgctgcccaaaggagggggcgtataggcaggaatccgaacaaacctggtgatgagtatctatatggaggtggttgcgcagagacggacgaagaccatgcgcactggcttgaagcgagaatgctcctagacaatatttatctccaagatggtctcatagcctcgctatatcgacctgaagccgacaaagtagcggccattgagggtgagttcaagctaaggacggagcaacgtaagacctttgtagaactcatgaaaagaggtgatcttcctgtatggctggcctatcaagttgcatctgcgggaataacctatacagatagaagatggtgttttgatggcacgacgaacaacaccataatggaagattcggtgccggcagaagtgtggaccagacatggagagaaacgtgtgctcaaaccgaggtggatggatgccagagtttgttcagatcatgcggcgctgaagtcatttaaggagtttgcggctgggaaaagaggtgcggcttttggtgtaatggaagccctgggaacactgccgggacacatgacggagagattccaagaagccattgataacctcgctgtactcatgcgggcggagactggaagtaggccttacaaagccgcggcggcgcaattgccggaaaccctagagacgataatgcttttagggttgctgggtacagtctcgctaggaatcttctttgtcttgatgcgtaacaagggcataggtaagatgggctttggtatggtgactctaggggccagcgcgtggctcatgtggctatcggaaattgaaccagccagaatagcatgtgtcctaattgttgtattcctattgctggtagtactcatacctgaaccagaaaagcaacgttctccccaggataaccaaatggcaataatcatcatggtagcggtaggtcttctaggcttgattacggccaatgaactaggatggttggaaagaacaaagtcggacctaagccatctaatgggtaggagagaggaaggggcaaccataggtttctcaatggatattgacctgcgtccagcctcagcgtgggccatctatgcggccttgacaacgttcattaccccggccgtccaacatgcggtgaccacctcgtacaacaactattccttaatggcgatggcgacgcaagctggtgtgttgtttggtatgggtaaagggatgccgttctacgcatgggattttggagtcccgctactaatgataggttgttactcacaattaacgcccctgaccctaatagtagccatcattttactcgtggcgcattacatgtacttaatcccagggctacaggcagcagcggcgcgtgctgcgcagaagagaacggcggctggcatcatgaaaaaccctgttgtagatggaatagtagtgactgacatagacacaatgacgattgacccccaagtagagaaaaagatgggtcaggtgctactaatagcagtagcggtctccagcgcgatactgtcgcggaccgcctggggttggggggaggcgggggccctgataacagccgcaacgtccactttgtgggaaggttctccgaacaaatactggaactcgtctacagccacgtcactgtgtaatatttttaggggtagttacttggcgggagcttctctaatatacacagtaacgagaaacgctggtttggtcaagcgtcgtgggggtggtacaggagagacgctgggagagaaatggaaagcccgcttgaatcagatgtcggcgctggagttctattcctacaaaaaatcaggcatcacggaggtgtgccgtgaagaggcccgtcgcgccctcaaagacggtgtggcgacgggaggccatgcggtgtcccgaggtagtgcaaagctaagatggttggtagagcggggatatctgcagccctatggtaaggtcattgatctaggatgtggccgtgggggctggtcgtactacgccgcgaccatccgcaaagtacaagaagtgaaaggttacacaaaaggtggccctggtcatgaagaacccgtgttggtgcaaagttatgggtggaatatagtccgtctaaagagtggggtagacgtctttcatatggcggcggagccgtgtgatacgctgctgtgtgatataggtgagtcgtcatctagtccggaagtggaagaagcgcggacgctccgtgtcctctccatggtaggggattggctagaaaaaagaccgggagccttttgtataaaagtattgtgcccatataccagcactatgatggaaacgctggagcgactacagcgtaggtatggtggaggactggtaagagtgccactatcccgcaactcgacacatgagatgtattgggtctctggtgcgaaaagcaataccataaaaagtgtatccaccacgtcgcagctcctcttagggcgcatggatgggcctagacgtccagtgaaatatgaagaggatgtgaatctaggctctggcacgcgtgctgtggtaagttgcgctgaagcgcccaacatgaaaatcattggtaatcgcattgaaaggatacgcagtgagcatgcggaaacgtggttttttgacgagaatcacccatataggacgtgggcttaccatggtagctatgaggcgcccacacaaggttcagcgtcctcgctaataaacggtgttgtcaggctactgtcaaaaccgtgggatgtggtaactggagtcacgggaatagccatgacggacaccacaccgtatggtcaacaaagagtttttaaggaaaaagtagacactagggtaccagacccccaagaaggtactcgtcaggtaatgagcatggtatcttcctggttatggaaagagctaggtaaacacaaacgtccacgagtctgtacgaaagaagagtttatcaacaaggtacgtagcaatgcggcattaggggcgatatttgaagaagaaaaagagtggaaaactgcagtggaagcggtgaacgatccgaggttctgggcgctagtggacaaagaaagagagcatcacctgagaggtgagtgccagtcgtgtgtgtataatatgatgggaaaacgtgaaaagaaacaaggtgaatttggaaaagccaagggcagccgtgccatctggtatatgtggctaggtgctagatttctagaattcgaagcccttggattcttgaatgaggatcactggatgggtagagagaactcgggaggtggtgtagaagggctgggtttacaaagactaggatatgtcctagaagaaatgagtcgtataccgggaggaaggatgtatgcggatgacactgcgggctgggacacgcgcattagccgttttgatctggaaaatgaagctctaataaccaaccaaatggaaaaagggcacagggcgttggcattggcgataatcaagtatacataccaaaataaagtggtaaaagtccttagaccggctgaaaaaggtaaaacagttatggatattatttcgcgtcaagaccaaaggggtagcggacaagtagtcacttacgcgcttaacacatttacgaacctagtggtacaactcattcgtaatatggaggcggaggaagttctagaaatgcaagacttatggctgctgcgtaggtcagagaaagtaaccaactggttacagagcaacggttgggataggctaaaacgaatggcggtcagtggagatgattgtgttgtgaagccgattgatgatcgttttgcacatgcgctcaggttcttaaatgatatgggtaaagttagaaaagacacacaagaatggaaaccctcgactggatgggataactgggaagaagtaccgttttgctcgcaccacttcaataagctccatctaaaggacgggcgttccattgtggtaccctgccgccatcaagatgaactaattggccgggcgcgcgtctctccgggggcgggatggtcgatccgggagacggcttgcctagcgaaatcatatgcgcaaatgtggcagctactttatttccatagaagggacctacgactgatggcgaatgccatttgttcgtctgtgccagtagactgggttccgactgggagaacgacctggtcaatacatggaaagggtgaatggatgacgactgaagacatgctagtggtgtggaatagagtgtggatagaggagaacgatcacatggaagataagaccccagtaacgaaatggacggacattccctatttaggaaaaagggaagatttgtggtgtggttctctcataggtcacagaccgcgtaccacctgggcggagaacattaaaaatacagtcaacatggtacgcaggatcataggtgatgaagaaaaatatatggactacctatcgacccaagttcgttacttgggtgaagaaggttctacacctggtgtgctgtaa

SEQ ID NO: 7 - DNA, 7890 bp, Artificial Sequence; identical to SEQ ID NO: 6
  Vaccine candidate ZIKV-DO-scattered sequence with deoptimized region, NS1 to NS5

SEQ ID NO: 8 - DNA, 3854 bp, Artificial Sequence
  Vaccine candidate ZIKV-DO nonstructural region nucleotide sequence, regions NS1 to NS3
gtcggttgttcggtagatttttcgaaaaaagaaacgcgatgtggtacgggtgtatttgtatataatgacgtagaagcgtggcgagaccgatacaagtatcatccggactcgccgcgacgattagcggcggcggtaaaacaagcgtgggaagacggtatatgcggtatatcgtcggtatcgcgaatggaaaatataatgtggcgatcggtagaaggtgagttaaatgcgatactagaagagaatggcgtacaactaacggtagtagtcggctcggtaaaaaatcccatgtggcgaggtccgcagcgattgcccgtccccgtcaatgagctaccccatggttggaaggcgtggggtaaatcgtacttcgtacgagcggcgaagacgaataattcctttgtagtcgatggtgacacgctaaaggaatgcccgttaaaacatcgagcgtggaattcctttttggtcgaggatcatggtttcggtgtattccataccagtgtatggttaaaggtacgagaagattattcgttagagtgtgatccggccgtaattggcacggcggtaaagggcaaggaggcggtacatagtgatctcggttactggattgagagtgagaagaatgacacgtggcgcctaaagcgcgcccatctaatagagatgaaaacgtgtgaatggccgaagtcgcacacgttgtggacggatggcatagaagagagtgatctaataatacccaagtcgttagcgggtccgttatcccatcataatacccgagagggttaccgcacccaaatgaaaggtccgtggcatagtgaagagttggaaattcggttcgaggaatgcccgggtaccaaggtacacgtcgaggaaacgtgtggcacgcgaggcccgtcgctacgatcgaccaccgcgtccggccgcgtcatagaggaatggtgctgccgcgagtgcacgatgcccccgctatcgttccgggcgaaagatggttgttggtatggaatggagatacgcccccgcaaagaaccggaatccaatttagtacgctcgatggtcaccgcgggctcgaccgatcatatggaccatttctcgttgggcgtcttggtcatactattaatggtccaagaaggtctaaagaagcgaatgaccacgaagataataatatccacgtcgatggcggtcctagtagcgatgatactaggcggcttttcgatgagtgacctagcgaagttggcgattttgatgggtgccaccttcgcggaaatgaataccggcggcgatgtagcgcatctagcgctaatagcggcgttcaaagtacgaccggcgttgctagtatcgttcatattccgagcgaattggacgccccgtgaatccatgctactagccttggcctcgtgtttgttgcaaaccgcgatatcggccttggaaggtgacctaatggtattaataaatggtttcgcgttggcctggttggcgatacgagcgatggtagtaccgcgcaccgataatataaccttggcgatactagcggcgctaacgccgctagcccggggtacgctattggtcgcgtggcgagcgggtttggcgacctgcggtggttttatgttattatcgctaaagggcaaaggtagtgtcaagaagaatttaccgtttgtaatggccctaggcctcaccgcggtccgcctagtagaccccataaatgtcgtcggcctactattattaacgcgcagtggtaagcggtcctggcccccctccgaagtattaacggcggtaggtctaatatgcgcgttggcgggcggtttcgccaaggcggatatagagatggcgggtcccatggccgcggtaggtctactcattgtaagttacgtcgtatcgggcaagagtgtcgacatgtacattgaacgagcgggtgacataacgtgggaaaaagatgcggaagtaaccggcaatagtccccggttagatgtcgcgctcgatgagagtggtgatttctcgctagtcgaggatgacggtcccccgatgcgagagataatattaaaggtcgtactaatgaccatatgtggtatgaatccgatagccatacccttcgcggcgggcgcgtggtacgtatacgtcaagaccggcaaacgcagtggtgcgctctgggatgtccccgcgcccaaggaagtaaaaaagggtgagaccacggatggcgtctaccgagtaatgacccgtcgactactcggttcgacgcaagtaggcgtcggcgtaatgcaagagggtgtattccacaccatgtggcatgtaacgaaaggctcggcgctacgatccggtgaaggtcgattggatccgtactggggcgatgtaaagcaagatctagtctcgtactgtggtccgtggaagctcgatgccgcctgggacggtcactccgaggtccagttattggccgtccccccgggcgagcgagcgcgcaatatacaaactctacccggcatattcaagacgaaggatggtgacattggcgcggtagcgctagattacccggcgggcacttcgggctcgccgatactcgacaagtgtggtcgagtcataggcttgtatggtaatggtgtagtcataaaaaatggtagttatgtaagtgccataacccaaggtcgccgcgaagaagagacccccgtagagtgcttcgagccctcgatgctaaagaagaagcaactcactgtattagacttgcatcccggcgcgggtaaaacccgccgagtattgcccgaaatagtacgtgaagccataaaaacgcgattacgtaccgtcatattagcgccgacccgcgtagtagcggcggaaatggaggaggccttgcgaggtttgccggtccgttatatgacgacggcggtaaatgtaacccattcgggcacggaaatagtagacttaatgtgccatgccaccttcacctcgcgtctcctccagccgatacgagtacccaattataatctatatattatggatgaggcccatttcacggatccctcgagtatagcggcgcgaggctacatttcgacgcgcgtagagatgggtgaggcggcggccatattcatgaccgccacgccgccgggcacccgtgacgcgttcccggactcgaattcgccgattatggacaccgaagtcgaagtaccggagcgagcctggtcctcgggttttgattgggtcacggatcattcgggcaaaacggtatggttcgtaccgtccgtccgcaatggtaatgagatagcggcgtgtctaacgaaggcgggcaaacgggtaatacagttatcccgaaagaccttcgagacggagttccaaaaaacgaaacatcaagagtgggacttcgtagtcacgaccgacatttcggagatgggtgccaatttcaaagcggaccgtgtaatagattcgcgccgatgcctcaagccggtaatattggatggtgagcgagtaattctagcgggccccatgcccgtaacgcatgcctccgcggcccaacgccgcggtcgcataggtcgcaatcccaataaacccggcgatgagtatctatatggcggtggttgcgcggagaccgacgaagaccatgcgcattggttggaagcgcgaatgttattggacaatatttacttacaagatggtttaatagcctcgttatatcgacccgaggccgacaaagtagcggccattgagggcgagttcaagttgcgcacggagcaacgcaagaccttcgtcgaattaatgaaacgaggcgatttgcccgtatggctagcctatcaagtagcgtcggccggcataacctacacggatcgacgatggtgcttcgatggtacgaccaataataccataatggaagatagtgtgccggc

SEQ ID NO: 9 - DNA, 7887 bp, Artificial Sequence
  Vaccine candidate ZIKV-DO sequence, with the deoptimized nonstructural regions NS1 to NS5
gtcggttgttcggtagatttttcgaaaaaagaaacgcgatgtggtacgggtgtatttgtatataatgacgtagaagcgtggcgagaccgatacaagtatcatccggactcgccgcgacgattagcggcggcggtaaaacaagcgtgggaagacggtatatgcggtatatcgtcggtatcgcgaatggaaaatataatgtggcgatcggtagaaggtgagttaaatgcgatactagaagagaatggcgtacaactaacggtagtagtcggctcggtaaaaaatcccatgtggcgaggtccgcagcgattgcccgtccccgtcaatgagctaccccatggttggaaggcgtggggtaaatcgtacttcgtacgagcggcgaagacgaataattcctttgtagtcgatggtgacacgctaaaggaatgcccgttaaaacatcgagcgtggaattcctttttggtcgaggatcatggtttcggtgtattccataccagtgtatggttaaaggtacgagaagattattcgttagagtgtgatccggccgtaattggcacggcggtaaagggcaaggaggcggtacatagtgatctcggttactggattgagagtgagaagaatgacacgtggcgcctaaagcgcgcccatctaatagagatgaaaacgtgtgaatggccgaagtcgcacacgttgtggacggatggcatagaagagagtgatctaataatacccaagtcgttagcgggtccgttatcccatcataatacccgagagggttaccgcacccaaatgaaaggtccgtggcatagtgaagagttggaaattcggttcgaggaatgtccgggtaccaaggtacacgtcgaggaaacgtgtggcacgcgaggcccgtcgctacgatcgaccaccgcgtccggccgcgtcatagaggaatggtgctgccgcgagtgcacgatgcccccgctatcgttccgggcgaaagatggttgttggtatggaatggagatacgcccccgcaaagaaccggaatccaatttagtacgctcgatggtcaccgcgggctcgaccgatcatatggaccatttctcgttgggcgtcttggtcatactattaatggtccaagaaggtctaaagaagcgaatgaccacgaagataataatatccacgtcgatggcggtcctagtagcgatgatactaggcggcttttcgatgagtgacctagcgaagttggcgattttgatgggtgccaccttcgcggaaatgaataccggcggcgatgtagcgcatctagcgctaatagcggcgttcaaagtacgaccggcgttgctagtatcgttcatattccgagcgaattggacgccccgtgaatccatgctactagccttggcctcgtgtttgttgcaaaccgcgatatcggccttggaaggtgacctaatggtattaataaatggtttcgcgttggcctggttggcgatacgagcgatggtagtaccgcgcaccgataatataaccttggcgatactagcggcgctaacgccgctagcccggggtacgctattggtcgcgtggcgagcgggtttggcgacctgcggtggttttatgttattatcgctaaagggcaaaggtagtgtcaagaagaatttaccgtttgtaatggccctaggcctcaccgcggtccgcctagtagaccccataaatgtcgtcggcctactattattaacgcgcagtggtaagcggtcctggcccccctccgaagtattaacggcggtaggtctaatatgcgcgttggcgggcggtttcgccaaggcggatatagagatggcgggtcccatggccgcggtaggtctactcattgtaagttacgtcgtatcgggcaagagtgtcgacatgtacattgaacgagcgggtgacataacgtgggaaaaagatgcggaagtaaccggcaatagtccccggttagatgtcgcgctcgatgagagtggtgatttctcgctagtcgaggatgacggtcccccgatgcgagagataatattaaaggtcgtactaatgaccatatgtggtatgaatccgatagccatacccttcgcggcgggcgcgtggtacgtatacgtcaagaccggcaaacgcagtggtgcgctctgggatgtccccgcgcccaaggaagtaaaaaagggtgagaccacggatggcgtctaccgagtaatgacccgtcgactactcggttcgacgcaagtaggcgtcggcgtaatgcaagagggtgtattccacaccatgtggcatgtaacgaaaggctcggcgctacgatccggtgaaggtcgattggatccgtactggggcgatgtaaagcaagatctagtctcgtactgtggtccgtggaagctcgatgccgcctgggacggtcactccgaggtccagttattggccgtcccgccgggcgagcgagcgcgcaatatacaaactctacccggcatattcaagacgaaggatggtgacattggcgcggtagcgctagattacccggcgggcacttcgggctcgccgatactcgacaagtgtggtcgagtcataggcttgtatggtaatggtgtagtcataaaaaatggtagttatgtaagtgccataacccaaggtcgccgcgaagaagagacccccgtagagtgcttcgagccctcgatgctaaagaagaagcaactcactgtattagacttgcatcccggcgcgggtaaaacccgccgagtattgcccgaaatagtacgtgaagccataaaaacgcgattacgtaccgtcatattagcgccgacccgcgtagtagcggcggaaatggaggaggccttgcgaggtttgccggtccgttatatgacgacggcggtaaatgtaacccattcgggcacggaaatagtagacttaatgtgccatgccaccttcacctcgcgtctcctccagccgatacgagtacccaattataatctatatattatggatgaggcccatttcacggatccctcgagtatagcggcgcgaggctacatttcgacgcgcgtagagatgggtgaggcggcggccatattcatgaccgccacgccgccgggcacccgtgacgcgttcccggactcgaattcgccgattatggacaccgaagtcgaagtaccggagcgagcctggtcctcgggttttgattgggtcacggatcattcgggcaaaacggtatggttcgtaccgtccgtccgcaatggtaatgagatagcggcgtgtctaacgaaggcgggcaaacgggtaatacagttatcccgaaagaccttcgagacggagttccaaaaaacgaaacatcaagagtgggacttcgtagtcacgaccgacatttcggagatgggtgccaatttcaaagcggaccgtgtaatagattcgcgccgatgcctcaagccggtaatattggatggtgagcgagtaattctagcgggccccatgcccgtaacgcatgcctccgcggcccaacgccgcggtcgcataggtcgcaatcccaataaacccggcgatgagtatctatatggcggtggttgcgcggagaccgacgaagaccatgcgcattggttggaagcgcgaatgttattggacaatatttacttacaagatggtttaatagcctcgttatatcgacccgaggccgacaaagtagcggccattgagggcgagttcaagttgcgcacggagcaacgcaagaccttcgtcgaattaatgaaacgaggcgatttgcccgtatggctagcctatcaagtagcgtcggcaggtataacctacacggatcgacgatggtgcttcgatggtacgaccaataataccataatggaagatagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccataatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgctgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctagaaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtataacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttagaaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcagatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaa

SEQ ID NO: 10 - DNA, 10807 bp, Artificial Sequence
  Vaccine candidate ZIKV-DO-NS3, more extensive sequence of flanking regions
agttgttgatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagttgggaaaaaagaggctatggaaataataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggggtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgatcctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttactcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaatccaatagccataccctttgcagctggagcgtggtacgtatacgtgaagactggaaaaaggagtggtgcgctctgggatgtccccgcgcccaaggaagtaaaaaagggtgagaccacggatggcgtctaccgagtaatgacccgtcgactactcggttcgacgcaagtaggcgtcggcgtaatgcaagagggtgtattccacaccatgtggcatgtaacgaaaggctcggcgctacgatccggtgaaggtcgattggatccgtactggggcgatgtaaagcaagatctagtctcgtactgtggtccgtggaagctcgatgccgcctgggacggtcactccgaggtccagttattggccgtcccgccgggcgagcgagcgcgcaatatacaaactctacccggcatattcaagacgaaggatggtgacattggcgcggtagcgctagattacccggcgggcacttcgggctcgccgatactcgacaagtgtggtcgagtcataggcttgtatggtaatggtgtagtcataaaaaatggtagttatgtaagtgccataacccaaggtcgccgcgaagaagagacccccgtagagtgcttcgagccctcgatgctaaagaagaagcaactcactgtattagacttgcatcccggcgcgggtaaaacccgccgagtattgcccgaaatagtacgtgaagccataaaaacgcgattacgtaccgtcatattagcgccgacccgcgtagtagcggcggaaatggaggaggccttgcgaggtttgccagtccgttatatgacgacggcggtaaatgtaacccattcgggcacggaaatagtagacttaatgtgccatgccaccttcacctcgcgtctcctccagccgatacgagtacccaattataatctatatattatggatgaggcccatttcacggatccctcgagtatagcggcgcgaggctacatttcgacgcgcgtagagatgggtgaggcggcggccatattcatgaccgccacgccgccgggcacccgtgacgcgttcccggactcgaattcgccgattatggacaccgaagtcgaagtaccggagcgagcctggtcctcgggttttgattgggtcacggatcattcgggcaaaacggtatggttcgtaccgtccgtccgcaatggtaatgagatagcggcgtgtctaacgaaggcgggcaaacgggtaatacagttatcccgaaagaccttcgagacggagttccaaaaaacgaaacatcaagagtgggacttcgtagtcacgaccgacatttcggagatgggtgccaatttcaaagcagaccgtgtaatagattcgcgccgatgcctcaagccggtaatattggatggtgagcgagtaattctagcgggccccatgcccgtaacgcatgcctccgcggcccaacgccgcggtcgcataggtcgcaatcccaataaacccggcgatgagtatctatatggcggtggttgcgcggagaccgacgaagaccatgcgcattggttggaagcgcgaatgttattggacaatatttacttacaagatggtttaatagcctcgttatatcgacccgaggccgacaaagtagcggccattgagggcgagttcaagttgcgcacggagcaacgcaagaccttcgtcgaattaatgaaacgaggcgatttgcccgtatggctagcctatcaagtagcgtcggcaggtataacctacacggatcgacgatggtgcttcgatggtacgaccaataataccataatggaagatagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccataatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgctgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctagaaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtataacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttagaaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcagatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcctgtgaccccccaggagaagctgggaaaccaagcctatagtcaggccgagaacgccatggcacggaagaagccatgctgcctgtgagcccctcagaggatactgagtcaaaaaaccccacgcgcttggaggcgcaggatgggaaaagaaggtggcgaccttccccacccttcaatctggggcctgaactggagatcagctgtggatccccagaagagggactagtggttagaggagaccccccggaaaacgcaaaacagcatattgacgctgggaaagaccagagactccatgagtttccaccacgctggccgccaggcacagatcgccgaacttcggcggccggtgtggggaaatccatggtttct

SEQ ID NO: 11 - DNA, 10807 bp, Artificial Sequence
  Vaccine candidate ZIKV-DO-scattered, more extensive sequence of flanking regions
agttgttgatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagttgggaaaaaagaggctatggaaataataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggggtgttgatcttcttatccacagccgtctctgctgatgtagggtgctcggtagacttctcaaagaaggaaacgagatgcggtacgggggtattcgtctataacgatgttgaagcctggcgtgacaggtacaaataccatcctgattccccccgtcgattggcagcagcggtcaagcaagcgtgggaagatggtatatgcgggatctcgtctgtttcacgtatggaaaacataatgtggagatcggtagaaggggagctaaacgcaatcctagaagagaatggtgttcaactgacggtagttgtaggatctgtaaaaaacccgatgtggagaggtccgcagagattgccggtacctgtaaacgagctgccccacggttggaaggcttggggtaaatcgtacttcgtaagagcagcaaaaacaaataactcgtttgtcgtggatggtgatacactgaaggaatgtccactcaaacatcgtgcatggaactcgtttcttgtagaggatcatggtttcggggtatttcatactagtgtctggctaaaggttagagaagattattcgttagagtgtgatccggccgttattggtacagctgttaaaggaaaggaggcggtacacagtgatctaggttactggattgaaagtgagaagaatgatacatggaggctaaagagggcccatctaatcgagatgaaaacgtgtgaatggccgaagtcccacacgttgtggacagatggtatagaagagtcggatctgatcataccgaagtctttagcggggccactcagtcatcacaatacgagagagggctataggacccaaatgaaaggtccatggcactcggaagagcttgaaatacggtttgaggaatgtccaggcactaaagtccacgtggaagaaacatgtggtacaagaggaccgtctctgagatcgaccactgcaagtggaagggtaatcgaggaatggtgttgcagggagtgcacgatgcccccactatcgttccgggcgaaagatggctgttggtatggtatggagatacgtcccaggaaagaaccggaaagcaacttagtacgttcaatggtaactgcaggatcgactgatcacatggatcacttctcccttggagtacttgtaatcctgctcatggtacaggaagggctaaagaagagaatgacgacaaagatcataataagcacatcgatggcagtactggtagctatgatactgggaggattttcgatgagtgacctagctaagcttgcgattttgatgggtgcgaccttcgcggaaatgaatactggaggagatgtagcgcatctggcgctaatagcggcatttaaagtcagaccggcgttgctggtatcgttcatcttccgtgctaattggacgccccgtgaatcgatgctgctggcgttggcctcgtgtctattgcaaactgcgatatccgccttggaaggtgacctgatggtactcatcaatggttttgcgttggcctggttagcaatacgagcgatggtagttccacgcacggataacatcacgttggcaatcctagctgctctgacgccactggcccgtggcacactgcttgtagcgtggagagcgggccttgctacgtgcggggggtttatgctactctctctgaaaggaaaaggcagtgtaaagaagaacttaccgtttgtcatggcgctgggactaacggctgtaaggctggtcgatcccatcaacgtagtaggactgctgttactaacaaggagtgggaaacggagctggccgcctagcgaagtactaacagctgttggtctgatatgcgcattggcgggagggttcgcgaaggcagatatagaaatggctgggccgatggccgcggtaggtctgctaatagtcagttacgtagtctcaggaaaaagtgtggacatgtatattgaaagagcgggtgacatcacatgggaaaaagatgcggaagtaactggaaacagtccgcggctcgatgtagcgctagatgaaagtggtgatttttccctggtagaggatgacggtccccccatgagagaaatcatactcaaagtagtcctgatgacgatctgtggcatgaatccgatagccataccgtttgcagctggtgcgtggtacgtatacgtaaagactggaaaacgtagtggtgctctatgggatgtacctgctcccaaagaagtaaaaaaaggggagaccacggatggagtatacagagtaatgacgcgtagactgctaggttcgacacaagttggtgtaggagttatgcaagaaggggtctttcatactatgtggcatgtcacaaaaggttccgcgctgcgtagcggtgaaggtagacttgatccgtactggggagatgtaaagcaggatctagtatcatactgtggtccgtggaagctagatgcggcctgggacggtcacagcgaggtacagctcttggcggtaccccccggagaaagagcgaggaatatccagactctacccggaatatttaaaacaaaggatggtgacattggagcggtagcgctggattatccagcaggaacgtcaggatctccgatcctagacaaatgtgggagagtaataggactttatggtaatggggtcgtaatcaaaaatggtagttatgttagtgcgatcacccaaggtaggagggaagaagaaactcctgttgaatgcttcgagccgtcgatgctgaaaaagaagcagctaacggtcttagacttacatcctggagcggggaaaacccgaagagttcttccggaaatagtccgtgaagcgataaaaacacgtctccgtactgtaatcttagctccgaccagggttgtagctgctgaaatggaagaggcccttcgtgggcttccagtacgttatatgacgacagcagtcaatgtaacccactctggtacagaaatcgttgacttaatgtgtcatgccacctttacttcacgtctactacaaccaatcagagttcccaactataatctatatattatggatgaagcccacttcacggatccctcaagtatagcggcaagaggatatatttcaacaagggttgaaatgggcgaggcggcggccatcttcatgacggccacgccaccgggaacccgtgatgcatttccggattccaactcaccgattatggacacggaagtggaagttccagagagagcgtggagctcaggttttgattgggtaacggatcattcgggaaaaacagtttggtttgttccgagcgtgaggaatggcaatgagatagcagcttgtctaacaaaggctggtaaacgggtcatacaactcagcagaaaaacttttgaaacagagttccaaaaaacaaaacatcaagaatgggactttgttgttacaactgacatatcagagatgggtgccaactttaaagctgaccgtgtcatagattcgaggagatgcctaaagccggtcatactagatggcgagcgagtcattctggcgggacccatgccggtcacacatgcgagcgctgcccaaaggagggggcgtataggcaggaatccgaacaaacctggtgatgagtatctatatggaggtggttgcgcagagacggacgaagaccatgcgcactggcttgaagcgagaatgctcctagacaatatttatctccaagatggtctcatagcctcgctatatcgacctgaagccgacaaagtagcggccattgagggtgagttcaagctaaggacggagcaacgtaagacctttgtagaactcatgaaaagaggtgatcttcctgtatggctggcctatcaagttgcatctgcgggaataacctatacagatagaagatggtgttttgatggcacgacgaacaacaccataatggaagattcggtgccggcagaagtgtggaccagacatggagagaaacgtgtgctcaaaccgaggtggatggatgccagagtttgttcagatcatgcggcgctgaagtcatttaaggagtttgcggctgggaaaagaggtgcggcttttggtgtaatggaagccctgggaacactgccgggacacatgacggagagattccaagaagccattgataacctcgctgtactcatgcgggcggagactggaagtaggccttacaaagccgcggcggcgcaattgccggaaaccctagagacgataatgcttttagggttgctgggtacagtctcgctaggaatcttctttgtcttgatgcgtaacaagggcataggtaagatgggctttggtatggtgactctaggggccagcgcgtggctcatgtggctatcggaaattgaaccagccagaatagcatgtgtcctaattgttgtattcctattgctggtagtactcatacctgaaccagaaaagcaacgttctccccaggataaccaaatggcaataatcatcatggtagcggtaggtcttctaggcttgattacggccaatgaactaggatggttggaaagaacaaagtcggacctaagccatctaatgggtaggagagaggaaggggcaaccataggtttctcaatggatattgacctgcgtccagcctcagcgtgggccatctatgcggccttgacaacgttcattaccccggccgtccaacatgcggtgaccacctcgtacaacaactattccttaatggcgatggcgacgcaagctggtgtgttgtttggtatgggtaaagggatgccgttctacgcatgggattttggagtcccgctactaatgataggttgttactcacaattaacgcccctgaccctaatagtagccatcattttactcgtggcgcattacatgtacttaatcccagggctacaggcagcagcggcgcgtgctgcgcagaagagaacggcggctggcatcatgaaaaaccctgttgtagatggaatagtagtgactgacatagacacaatgacgattgacccccaagtagagaaaaagatgggtcaggtgctactaatagcagtagcggtctccagcgcgatactgtcgcggaccgcctggggttggggggaggcgggggccctgataacagccgcaacgtccactttgtgggaaggttctccgaacaaatactggaactcgtctacagccacgtcactgtgtaatatttttaggggtagttacttggcgggagcttctctaatatacacagtaacgagaaacgctggtttggtcaagcgtcgtgggggtggtacaggagagacgctgggagagaaatggaaagcccgcttgaatcagatgtcggcgctggagttctattcctacaaaaaatcaggcatcacggaggtgtgccgtgaagaggcccgtcgcgccctcaaagacggtgtggcgacgggaggccatgcggtgtcccgaggtagtgcaaagctaagatggttggtagagcggggatatctgcagccctatggtaaggtcattgatctaggatgtggccgtgggggctggtcgtactacgccgcgaccatccgcaaagtacaagaagtgaaaggttacacaaaaggtggccctggtcatgaagaacccgtgttggtgcaaagttatgggtggaatatagtccgtctaaagagtggggtagacgtctttcatatggcggcggagccgtgtgatacgctgctgtgtgatataggtgagtcgtcatctagtccggaagtggaagaagcgcggacgctccgtgtcctctccatggtaggggattggctagaaaaaagaccgggagccttttgtataaaagtattgtgcccatataccagcactatgatggaaacgctggagcgactacagcgtaggtatggtggaggactggtaagagtgccactatcccgcaactcgacacatgagatgtattgggtctctggtgcgaaaagcaataccataaaaagtgtatccaccacgtcgcagctcctcttagggcgcatggatgggcctagacgtccagtgaaatatgaagaggatgtgaatctaggctctggcacgcgtgctgtggtaagttgcgctgaagcgcccaacatgaaaatcattggtaatcgcattgaaaggatacgcagtgagcatgcggaaacgtggttttttgacgagaatcacccatataggacgtgggcttaccatggtagctatgaggcgcccacacaaggttcagcgtcctcgctaataaacggtgttgtcaggctactgtcaaaaccgtgggatgtggtaactggagtcacgggaatagccatgacggacaccacaccgtatggtcaacaaagagtttttaaggaaaaagtagacactagggtaccagacccccaagaaggtactcgtcaggtaatgagcatggtatcttcctggttatggaaagagctaggtaaacacaaacgtccacgagtctgtacgaaagaagagtttatcaacaaggtacgtagcaatgcggcattaggggcgatatttgaagaagaaaaagagtggaaaactgcagtggaagcggtgaacgatccgaggttctgggcgctagtggacaaagaaagagagcatcacctgagaggtgagtgccagtcgtgtgtgtataatatgatgggaaaacgtgaaaagaaacaaggtgaatttggaaaagccaagggcagccgtgccatctggtatatgtggctaggtgctagatttctagaattcgaagcccttggattcttgaatgaggatcactggatgggtagagagaactcgggaggtggtgtagaagggctgggtttacaaagactaggatatgtcctagaagaaatgagtcgtataccgggaggaaggatgtatgcggatgacactgcgggctgggacacgcgcattagccgttttgatctggaaaatgaagctctaataaccaaccaaatggaaaaagggcacagggcgttggcattggcgataatcaagtatacataccaaaataaagtggtaaaagtccttagaccggctgaaaaaggtaaaacagttatggatattatttcgcgtcaagaccaaaggggtagcggacaagtagtcacttacgcgcttaacacatttacgaacctagtggtacaactcattcgtaatatggaggcggaggaagttctagaaatgcaagacttatggctgctgcgtaggtcagagaaagtaaccaactggttacagagcaacggttgggataggctaaaacgaatggcggtcagtggagatgattgtgttgtgaagccgattgatgatcgttttgcacatgcgctcaggttcttaaatgatatgggtaaagttagaaaagacacacaagaatggaaaccctcgactggatgggataactgggaagaagtaccgttttgctcgcaccacttcaataagctccatctaaaggacgggcgttccattgtggtaccctgccgccatcaagatgaactaattggccgggcgcgcgtctctccgggggcgggatggtcgatccgggagacggcttgcctagcgaaatcatatgcgcaaatgtggcagctactttatttccatagaagggacctacgactgatggcgaatgccatttgttcgtctgtgccagtagactgggttccgactgggagaacgacctggtcaatacatggaaagggtgaatggatgacgactgaagacatgctagtggtgtggaatagagtgtggatagaggagaacgatcacatggaagataagaccccagtaacgaaatggacggacattccctatttaggaaaaagggaagatttgtggtgtggttctctcataggtcacagaccgcgtaccacctgggcggagaacattaaaaatacagtcaacatggtacgcaggatcataggtgatgaagaaaaatatatggactacctatcgacccaagttcgttacttgggtgaagaaggttctacacctggtgtgctgtaagcaccaatcttaatgttgtcaggcctgctagtcagccacagcttggggaaagctgtgcagcctgtgaccccccaggagaagctgggaaaccaagcctatagtcaggccgagaacgccatggcacggaagaagccatgctgcctgtgagcccctcagaggatactgagtcaaaaaaccccacgcgcttggaggcgcaggatgggaaaagaaggtggcgaccttccccacccttcaatctggggcctgaactggagatcagctgtggatccccagaagagggactagtggttagaggagaccccccggaaaacgcaaaacagcatattgacgctgggaaagaccagagactccatgagtttccaccacgctggccgccaggcacagatcgccgaacttcggcggccggtgtggggaaatccatggtttct

SEQ ID NO: 12 - DNA, 10807 bp, Artificial Sequence
  Vaccine candidate ZIKV-DO, more extensive sequence of flanking regions
agttgttgatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagttgggaaaaaagaggctatggaaataataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggggtgttgatcttcttatccacagccgtctctgctgatgtcggttgttcggtagatttttcgaaaaaagaaacgcgatgtggtacgggtgtatttgtatataatgacgtagaagcgtggcgagaccgatacaagtatcatccggactcgccgcgacgattagcggcggcggtaaaacaagcgtgggaagacggtatatgcggtatatcgtcggtatcgcgaatggaaaatataatgtggcgatcggtagaaggtgagttaaatgcgatactagaagagaatggcgtacaactaacggtagtagtcggctcggtaaaaaatcccatgtggcgaggtccgcagcgattgcccgtccccgtcaatgagctaccccatggttggaaggcgtggggtaaatcgtacttcgtacgagcggcgaagacgaataattcctttgtagtcgatggtgacacgctaaaggaatgcccgttaaaacatcgagcgtggaattcctttttggtcgaggatcatggtttcggtgtattccataccagtgtatggttaaaggtacgagaagattattcgttagagtgtgatccggccgtaattggcacggcggtaaagggcaaggaggcggtacatagtgatctcggttactggattgagagtgagaagaatgacacgtggcgcctaaagcgcgcccatctaatagagatgaaaacgtgtgaatggccgaagtcgcacacgttgtggacggatggcatagaagagagtgatctaataatacccaagtcgttagcgggtccgttatcccatcataatacccgagagggttaccgcacccaaatgaaaggtccgtggcatagtgaagagttggaaattcggttcgaggaatgtccgggtaccaaggtacacgtcgaggaaacgtgtggcacgcgaggcccgtcgctacgatcgaccaccgcgtccggccgcgtcatagaggaatggtgctgccgcgagtgcacgatgcccccgctatcgttccgggcgaaagatggttgttggtatggaatggagatacgcccccgcaaagaaccggaatccaatttagtacgctcgatggtcaccgcgggctcgaccgatcatatggaccatttctcgttgggcgtcttggtcatactattaatggtccaagaaggtctaaagaagcgaatgaccacgaagataataatatccacgtcgatggcggtcctagtagcgatgatactaggcggcttttcgatgagtgacctagcgaagttggcgattttgatgggtgccaccttcgcggaaatgaataccggcggcgatgtagcgcatctagcgctaatagcggcgttcaaagtacgaccggcgttgctagtatcgttcatattccgagcgaattggacgccccgtgaatccatgctactagccttggcctcgtgtttgttgcaaaccgcgatatcggccttggaaggtgacctaatggtattaataaatggtttcgcgttggcctggttggcgatacgagcgatggtagtaccgcgcaccgataatataaccttggcgatactagcggcgctaacgccgctagcccggggtacgctattggtcgcgtggcgagcgggtttggcgacctgcggtggttttatgttattatcgctaaagggcaaaggtagtgtcaagaagaatttaccgtttgtaatggccctaggcctcaccgcggtccgcctagtagaccccataaatgtcgtcggcctactattattaacgcgcagtggtaagcggtcctggcccccctccgaagtattaacggcggtaggtctaatatgcgcgttggcgggcggtttcgccaaggcggatatagagatggcgggtcccatggccgcggtaggtctactcattgtaagttacgtcgtatcgggcaagagtgtcgacatgtacattgaacgagcgggtgacataacgtgggaaaaagatgcggaagtaaccggcaatagtccccggttagatgtcgcgctcgatgagagtggtgatttctcgctagtcgaggatgacggtcccccgatgcgagagataatattaaaggtcgtactaatgaccatatgtggtatgaatccgatagccatacccttcgcggcgggcgcgtggtacgtatacgtcaagaccggcaaacgcagtggtgcgctctgggatgtccccgcgcccaaggaagtaaaaaagggtgagaccacggatggcgtctaccgagtaatgacccgtcgactactcggttcgacgcaagtaggcgtcggcgtaatgcaagagggtgtattccacaccatgtggcatgtaacgaaaggctcggcgctacgatccggtgaaggtcgattggatccgtactggggcgatgtaaagcaagatctagtctcgtactgtggtccgtggaagctcgatgccgcctgggacggtcactccgaggtccagttattggccgtcccgccgggcgagcgagcgcgcaatatacaaactctacccggcatattcaagacgaaggatggtgacattggcgcggtagcgctagattacccggcgggcacttcgggctcgccgatactcgacaagtgtggtcgagtcataggcttgtatggtaatggtgtagtcataaaaaatggtagttatgtaagtgccataacccaaggtcgccgcgaagaagagacccccgtagagtgcttcgagccctcgatgctaaagaagaagcaactcactgtattagacttgcatcccggcgcgggtaaaacccgccgagtattgcccgaaatagtacgtgaagccataaaaacgcgattacgtaccgtcatattagcgccgacccgcgtagtagcggcggaaatggaggaggccttgcgaggtttgccggtccgttatatgacgacggcggtaaatgtaacccattcgggcacggaaatagtagacttaatgtgccatgccaccttcacctcgcgtctcctccagccgatacgagtacccaattataatctatatattatggatgaggcccatttcacggatccctcgagtatagcggcgcgaggctacatttcgacgcgcgtagagatgggtgaggcggcggccatattcatgaccgccacgccgccgggcacccgtgacgcgttcccggactcgaattcgccgattatggacaccgaagtcgaagtaccggagcgagcctggtcctcgggttttgattgggtcacggatcattcgggcaaaacggtatggttcgtaccgtccgtccgcaatggtaatgagatagcggcgtgtctaacgaaggcgggcaaacgggtaatacagttatcccgaaagaccttcgagacggagttccaaaaaacgaaacatcaagagtgggacttcgtagtcacgaccgacatttcggagatgggtgccaatttcaaagcggaccgtgtaatagattcgcgccgatgcctcaagccggtaatattggatggtgagcgagtaattctagcgggccccatgcccgtaacgcatgcctccgcggcccaacgccgcggtcgcataggtcgcaatcccaataaacccggcgatgagtatctatatggcggtggttgcgcggagaccgacgaagaccatgcgcattggttggaagcgcgaatgttattggacaatatttacttacaagatggtttaatagcctcgttatatcgacccgaggccgacaaagtagcggccattgagggcgagttcaagttgcgcacggagcaacgcaagaccttcgtcgaattaatgaaacgaggcgatttgcccgtatggctagcctatcaagtagcgtcggcaggtataacctacacggatcgacgatggtgcttcgatggtacgaccaataataccataatggaagatagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccataatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgctgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctagaaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtataacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttagaaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcagatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcctgtgaccccccaggagaagctgggaaaccaagcctatagtcaggccgagaacgccatggcacggaagaagccatgctgcctgtgagcccctcagaggatactgagtcaaaaaaccccacgcgcttggaggcgcaggatgggaaaagaaggtggcgaccttccccacccttcaatctggggcctgaactggagatcagctgtggatccccagaagagggactagtggttagaggagaccccccggaaaacgcaaaacagcatattgacgctgggaaagaccagagactccatgagtttccaccacgctggccgccaggcacagatcgccgaacttcggcggccggtgtggggaaatccatggtttct